(19) United States
(12) Patent Application Publication
(10) Pub. No.: US 2023/0192676 A1
(43) Pub. Date: Jun. 22, 2023

(54) Heterocyclic Amides as Kinase Inhibitors

(71) Applicant: GLAXOSMITHKLINE INTELLECTUAL PROPERTY DEVELOPMENT LIMITED, BRENTFORD MIDDLESEX (GB)

(72) Inventors: Ryan Michael Fox, Collegeville, PA (US); Philip Anthony Harris, Collegeville, PA (US); Joerg Holenz, Bolton, MA (US); Mark Andrew Seefeld, Collegeville, PA (US); Ding Zhou, Shanghai (CN)

(21) Appl. No.: 16/958,659

(22) PCT Filed: Dec. 27, 2018
(86) PCT No.: PCT/IB2018/060641

§ 371 Date: Jun. 26, 2020

(30) Foreign Application Priority Data
Dec. 29, 2017 (WO) PCT/CN2017/120014

(51) Int. Cl. C07D 413/06; A61P 35/00; C07D 413/14; C07D 417/14

(52) U.S. Cl. CPC C07D 413/06; A61P 35/00; C07D 413/14; C07D 417/14

(57) ABSTRACT

Disclosed are compounds having the formula (I) wherein R¹, R², and R³ are as defined herein, and methods of making and using the same.

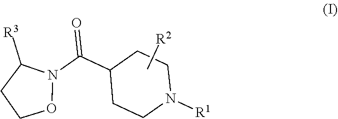

### FIELD OF THE INVENTION

The present invention relates to heterocyclic amides that inhibit RIP1 kinase and methods of making and using the same.

### BACKGROUND OF THE INVENTION

Receptor-interacting protein-1 (RIP1) kinase, originally referred to as RIP, is a TKL family serine/threonine protein kinase involved in innate immune signaling. RIP1 kinase is a RHIM domain containing protein, with an N-terminal kinase domain and a C-terminal death domain (Trends Biochem. Sci., 30, 151-159 (2005)). The death domain of RIP1 mediates interaction with other death domain containing proteins including Fas and TNFR-1 (Cell, 81 513-523 (1995)), TRAIL-R1 and TRAIL-R² (Immunity, 7, 821-830 (1997)), and TRADD (Immunity, 4, 387-396 (1996)), while the RHIM domain is crucial for binding other RHIM domain containing proteins such as TRIF (Nat. Immunol., 5, 503-507 (2004)), DAI (EMBO Rep. 10, 916-922 (2009)) and RIP3 (J. Biol. Chem., 274, 16871-16875 (1999)); Curr. Biol., 9, 539-542 (1999)) and exerts many of its effects through these interactions. RIP1 is a central regulator of cell signaling, and is involved in mediating both pro-survival and programmed cell death pathways which will be discussed below.

The role for RIP1 in cell signaling has been assessed under various conditions [including TLR3 (Nat Immunol., 5, 503-507 (2004)), TLR4 (J. Biol. Chem., 280, 36560-6566 (2005)), TRAIL (Cell Signal., 27(2), 306-314 (2015)), FAS (J. Biol. Chem., 279, 7925-7933 (2004))], but is best understood in the context of mediating signals downstream of the death receptor TNFR1 (Cell, 114, 181-190 (2003)). Engagement of the TNFR by TNF leads to its oligomerization, and the recruitment of multiple proteins, including linear K63-linked polyubiquitinated RIP1 (Mol. Cell, 22, 245-257 (2006)), TRAF2/5 (J. Mol. Biol., 396, 528-539 (2010)), TRADD (Nat. Immunol., 9, 1037-1046 (2008)), and cIAPs (Proc. Natl. Acad. Sci. USA., 105, 11778-11783 (2008)), to the cytoplasmic tail of the receptor. This complex which is dependent on RIP1 as a scaffolding protein (i.e. kinase independent), termed complex I, provides a platform for pro-survival signaling through the activation of the NFκB and MAP kinases pathways (Sci. Signal., 115, re4 (2010)). Alternatively, binding of TNF to its receptor under conditions promoting the deubiquitination of RIP1 (by proteins such as A20 and CYLD or inhibition of the cIAPs) results in receptor internalization and the formation of complex II or DISC (death-inducing signaling complex) (Cell Death Dis., 2, e230 (2011)). Formation of the DISC, which contains RIP1, TRADD, FADD and caspase 8, results in the activation of caspase 8 and the onset of programmed apoptotic cell death also in a RIP1 kinase independent fashion (FEBS J, 278, 877-887 (2012)). Apoptosis is largely a quiescent form of cell death, and is involved in routine processes such as development and cellular homeostasis.

Under conditions where the DISC forms and RIP3 is expressed, but apoptosis is inhibited (such as FADD/caspase 8 deletion, caspase inhibition, or viral infection), a third RIP1 kinase-dependent possibility exists. RIP3 can now enter this complex, become phosphorylated by RIP1 and initiate a caspase-independent programmed necrotic cell death through the activation of MLKL and PGAM5 (Cell, 148, 213-227 (2012)); (Cell, 148, 228-243 (2012)); (Proc. Natl. Acad. Sci. USA., 109, 5322-5327 (2012)). As opposed to apoptosis, programmed necrosis (not to be confused with passive necrosis which is not programmed) results in the release of danger associated molecular patterns (DAMPs) from the cell. These DAMPs are capable of providing a “danger signal” to surrounding cells and tissues, eliciting proinflammatory responses including inflammasome activation, cytokine production and cellular recruitment (Nat. Rev. Immunol., 8, 279-289 (2008)).

Dysregulation of RIP1 kinase-mediated programmed cell death has been linked to various inflammatory diseases, as demonstrated by use of the RIP3 knockout mouse (where RIP1-mediated programmed necrosis is completely blocked) and by Necrostatin-1 (a tool inhibitor of RIP1 kinase activity with poor oral bioavailability). The RIP3 knockout mouse has been shown to be protective in inflammatory bowel disease (including ulcerative colitis and Crohn's disease) (Nature, 477, 330-334 (2011)), psoriasis (Immunity, 35, 572-582 (2011)), retinal-detachment-induced photoreceptor necrosis (PNAS, 107, 21695-21700, (2010)), retinitis pigmentosa (Proc. Natl. Acad. Sci., 109:36, 14598-14603 (2012)), cerulein-induced acute pancreatits (Cell, 137, 1100-1111 (2009)), and sepsis/systemic inflammatory response syndrome (SIRS) (Immunity, 35, 908-918 (2011)). Necrostatin-1 has been shown to be effective in alleviating ischemic brain injury (Nat. Chem. Biol., 1, 112-119 (2005)), retinal ischemia/reperfusion injury (J. Neurosci. Res., 88, 1569-1576 (2010)), Huntington's disease (Cell Death Dis., 2 e115 (2011)), renal ischemia reperfusion injury (Kidney Int., 81, 751-761 (2012)), cisplatin induced kidney injury (Ren. Fail., 34, 373-377 (2012)), and traumatic brain injury (Neurochem. Res., 37, 1849-1858 (2012)). Other diseases or disorders regulated at least in part by RIP1-dependent apoptosis, necrosis or cytokine production include hematological and solid organ malignancies (Genes Dev., 27, 1640-1649 (2013)), bacterial infections and viral infections (Cell Host & Microbe, 15, 23-35 (2014)) (including, but not limited to, tuberculosis and influenza (Cell, 153, 1-14 (2013)) and Lysosomal storage diseases (particularly, Gaucher Disease, Nature Medicine Advance Online Publication, 19 Jan. 2014, doi:10.1038/nm.3449).

A potent, selective, small molecule inhibitor of RIP1 kinase activity would block RIP1-dependent cellular necrosis and thereby provide a therapeutic benefit in diseases or events associated with DAMPs, cell death, and/or inflammation.

### SUMMARY OF THE INVENTION

The invention is directed to compounds according to Formula (I) or pharmaceutically acceptable salt thereof:

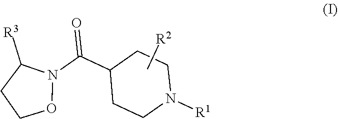

wherein:
R¹ is —CO(C₁-C₄)alkyl, —CO₂(C₁-C₄)alkyl, or 5-6 membered heteroaryl group, wherein said 5-6 membered heteroaryl group is optionally substituted by 1 or 2 substituents independently selected from cyano, (C₁-C₄)alkyl, —CONH₂, —CONH((C₁-C₄)alkyl), —CON((C₁-C₄)alkyl)((C₁-C₄)alkyl), —SO(C₁-C₄)alkyl, and —SO₂(C₁-C₄)alkyl;
R² is hydrogen or a halogen; and
R³ is phenyl or 5-6 membered heteroaryl group, wherein said phenyl or 5-6 membered heteroaryl group is optionally substituted by 1, 2, or 3 halogens.

The invention is further directed to compounds or pharmaceutically acceptable salts thereof according to Formula (II):

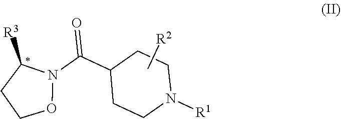

wherein:
R¹ is —CO(C₁-C₄)alkyl, —CO₂(C₁-C₄)alkyl, or 5-6 membered heteroaryl group, wherein said 5-6 membered heteroaryl group is optionally substituted by 1 or 2 substituents independently selected from cyano, (C₁-C₄)alkyl, —CONH₂, —CONH((C₁-C₄)alkyl), —CON((C₁-C₄)alkyl)((C₁-C₄)alkyl), —SO(C₁-C₄)alkyl, and —SO₂(C₁-C₄)alkyl;
R² is hydrogen or a halogen; and
R³ is phenyl or 5-6 membered heteroaryl group, wherein said phenyl or 5-6 membered heteroaryl group is optionally substituted by 1, 2, or 3 halogens.

The compounds according to Formulas (I) and (II), or pharmaceutically acceptable salts thereof, inhibit the activity and/or function of RIP1 kinase. Accordingly, these compounds may be particularly useful for the treatment of RIP1 kinase-mediated diseases or disorders. Such RIP1 kinase-mediated diseases or disorders are diseases or disorders that are mediated by activation of RIP1 kinase, and as such, are diseases or disorders where inhibition of RIP1 kinase would provide benefit.

### DETAILED DESCRIPTION OF THE INVENTION

This invention relates to compounds of Formulas (I) and (II) as defined above or pharmaceutically acceptable salts thereof.

In one embodiment, this invention relates to compounds of Formula (I) and Formula (II) wherein R¹ is —CO(C₁-C₄)alkyl or a 5-6 membered heteroaryl group, wherein said 5-6 membered heteroaryl group is optionally substituted by one substituent selected from cyano, (C₁-C₄)alkyl, —CONH₂, —CONH((C₁-C₄)alkyl), —CON((C₁-C₄)alkyl)((C₁-C₄)alkyl), —SO(C₁-C₄)alkyl, and —SO₂(C₁-C₄)alkyl. In another embodiment, the invention relates to compounds of Formula (I) and Formula (II) wherein R¹ is —CO(C₁-C₄)alkyl. In a specific embodiment, this invention relates to compounds of Formula (I) and Formula (II) wherein R¹ is —COCH₃. In another embodiment, the invention relates to compounds of Formula (I) and Formula (II) wherein R¹ is furyl, thienyl, pyrrolyl, imidazolyl, pyrazolyl, triazolyl, tetrazolyl, thiazolyl, isothiazolyl, thiadiazolyl, oxazolyl, isoxazolyl, oxadiazolyl, oxo-oxadiazolyl, pyridinyl, oxo-pyridyl, pyrazinyl, pyrimidinyl, pyridazinyl, or triazinyl, wherein said substituted furyl, thienyl, pyrrolyl, imidazolyl, pyrazolyl, triazolyl, tetrazolyl, thiazolyl, isothiazolyl, thiadiazolyl, oxazolyl, isoxazolyl, oxadiazolyl, oxo-oxadiazolyl, pyridinyl, oxo-pyridyl, pyrazinyl, pyrimidinyl, pyridazinyl and triazinyl is optionally substituted by one substituent selected from cyano, (C₁-C₄)alkyl, —CONH₂, —CONH((C₁-C₄)alkyl), —CON((C₁-C₄)alkyl)((C₁-C₄)alkyl), —SO(C₁-C₄)alkyl, and —SO₂(C₁-C₄)alkyl. In a further embodiment, the invention relates to compounds of Formula (I) and Formula (II) wherein R¹ is thiadiazolyl, oxadiazolyl, or pyrimidinyl, wherein thiadiazolyl, oxadiazolyl, or pyrimidinyl is optionally substituted by one substituent selected from cyano, (C₁-C₄)alkyl, —CONH₂, —CONH((C₁-C₄)alkyl), and —CON((C₁-C₄)alkyl)((C₁-C₄)alkyl). In a yet a further embodiment, the invention relates to compounds of Formula (I) and Formula (II) wherein R¹ is thiadiazolyl, oxadiazolyl, or pyrimidinyl, wherein thiadiazolyl, oxadiazolyl, or pyrimidinyl is optionally substituted by one substituent selected from cyano, methyl, —CONH₂, —CONHCH₃, and —CON(CH₃)₂. In a specific embodiment, the invention relates to compounds of Formula (I) and (II) wherein R¹ is pyrimidinyl substituted by —CONH₂. In another specific embodiment, the invention relates to compounds of Formula (I) and (II) wherein R¹ is pyrimidinyl substituted by —CONHCH₃. In another specific embodiment, the invention relates to compounds of Formula (I) and (II) wherein R¹ is pyrimidinyl substituted by —CON(CH₃)₂.

In one embodiment, this invention relates to compounds of Formula (I) and Formula (II) wherein R² is hydrogen or fluoro. In a specific embodiment, this invention relates to compounds of Formula (I) and Formula (II) wherein R² is fluoro. In another specific embodiment, this invention relates to compounds of Formula (I) and Formula (II) wherein R² is hydrogen.

In one embodiment, this invention relates to compounds of Formula (I) and Formula (II) wherein R³ is phenyl or 5-6 membered heteroaryl group, wherein said phenyl or 5-6 membered heteroaryl group is optionally substituted by one, two, or three fluoros. In another embodiment, this invention relates to compounds of Formula (I) and Formula (II) wherein R³ is phenyl, furyl, thienyl, pyrrolyl, imidazolyl, pyrazolyl, triazolyl, tetrazolyl, thiazolyl, isothiazolyl, thiadiazolyl, oxazolyl, isoxazolyl, oxadiazolyl, oxo-oxadiazolyl, pyridinyl, oxo-pyridyl, pyrazinyl, pyrimidinyl, pyridazinyl, or triazinyl, wherein said phenyl, furyl, thienyl, pyrrolyl, imidazolyl, pyrazolyl, triazolyl, tetrazolyl, thiazolyl, isothiazolyl, thiadiazolyl, oxazolyl, isoxazolyl, oxadiazolyl, oxo-oxadiazolyl, pyridinyl, oxo-pyridyl, pyrazinyl, pyrimidinyl, pyridazinyl, or triazinyl is optionally substituted by one, two, or three fluoros.

In another embodiment, this invention relates to compounds of Formula (I) and Formula (II) wherein R³ is phenyl or pyridinyl, wherein said phenyl or pyridinyl is optionally substituted by one, two, or three fluoros. In a specific embodiment, this invention relates to compounds of Formula (I) and Formula (II) wherein R³ is phenyl substituted by two fluoros. In yet another specific embodiment, this invention relates to compounds of Formula (I) and Formula (II) wherein R³ is pyridinyl substituted by one fluoro.

Specific compounds of this invention include:
1-(4-(3-(3,5-difluorophenyl)isoxazolidine-2-carbonyl)piperidin-1-yl)ethan-1-one;
(R)-1-(4-(3-(3,5-difluorophenyl)isoxazolidine-2-carbonyl)piperidin-1-yl)ethan-1-one;
(S)-1-(4-(3-(3,5-difluorophenyl)isoxazolidine-2-carbonyl)piperidin-1-yl)ethan-1-one;
6-(4-(3-(3,5-difluorophenyl)isoxazolidine-2-carbonyl)piperidin-1-yl)pyrimidine-4-carboxamide;
(R)-6-(4-(3-(3,5-difluorophenyl)isoxazolidine-2-carbonyl)piperidin-1-yl)pyrimidine-4-carboxamide;
(S)-6-(4-(3-(3,5-difluorophenyl)isoxazolidine-2-carbonyl)piperidin-1-yl)pyrimidine-4-carboxamide;
(3-(3,5-difluorophenyl)isoxazolidin-2-yl)(1-(5-methyl-1,3,4-oxadiazol-2-yl)piperidin-4-yl)methanone;
(R)-(3-(3,5-difluorophenyl)isoxazolidin-2-yl)(1-(5-methyl-1,3,4-oxadiazol-2-yl)piperidin-4-yl)methanone;
(S)-(3-(3,5-difluorophenyl)isoxazolidin-2-yl)(1-(5-methyl-1,3,4-oxadiazol-2-yl)piperidin-4-yl)methanone;
6-(4-(3-(3,5-difluorophenyl)isoxazolidine-2-carbonyl)piperidin-1-yl)pyrimidine-4-carbonitrile;
(R)-6-(4-(3-(3,5-difluorophenyl)isoxazolidine-2-carbonyl)piperidin-1-yl)pyrimidine-4-carbonitrile;
(S)-6-(4-(3-(3,5-difluorophenyl)isoxazolidine-2-carbonyl)piperidin-1-yl)pyrimidine-4-carbonitrile;
6-(4-(3-(3,5-difluorophenyl)isoxazolidine-2-carbonyl)piperidin-1-yl)-N-methylpyrimidine-4-carboxamide;
(R)-6-(4-(3-(3,5-difluorophenyl)isoxazolidine-2-carbonyl)piperidin-1-yl)-N-methylpyrimidine-4-carboxamide;
(S)-6-(4-(3-(3,5-difluorophenyl)isoxazolidine-2-carbonyl)piperidin-1-yl)-N-methylpyrimidine-4-carboxamide;
6-(4-(3-(3,5-difluorophenyl)isoxazolidine-2-carbonyl)piperidin-1-yl)-N,N-dimethylpyrimidine-4-carboxamide;
(R)-6-(4-(3-(3,5-difluorophenyl)isoxazolidine-2-carbonyl)piperidin-1-yl)-N,N-dimethylpyrimidine-4-carboxamide;
(S)-6-(4-(3-(3,5-difluorophenyl)isoxazolidine-2-carbonyl)piperidin-1-yl)-N,N-dimethylpyrimidine-4-carboxamide;
1-((3R,4S)-4-((S)-3-(3,5-difluorophenyl)isoxazolidine-2-carbonyl)-3-fluoropiperidin-1-yl)ethan-1-one;
1-((3S,4R)-4-((R)-3-(3,5-difluorophenyl)isoxazolidine-2-carbonyl)-3-fluoropiperidin-1-yl)ethan-1-one;
1-((3S,4R)-4-((S)-3-(3,5-difluorophenyl)isoxazolidine-2-carbonyl)-3-fluoropiperidin-1-yl)ethan-1-one;
1-((3R,4S)-4-((R)-3-(3,5-difluorophenyl)isoxazolidine-2-carbonyl)-3-fluoropiperidin-1-yl)ethan-1-one;
((S)-3-(3,5-difluorophenyl)isoxazolidin-2-yl)((3R,4S)-3-fluoro-1-(5-methyl-1,3,4-oxadiazol-2-yl)piperidin-4-yl)methanone;
((R)-3-(3,5-difluorophenyl)isoxazolidin-2-yl)((3S,4R)-3-fluoro-1-(5-methyl-1,3,4-oxadiazol-2-yl)piperidin-4-yl)methanone;
((S)-3-(3,5-difluorophenyl)isoxazolidin-2-yl)((3S,4R)-3-fluoro-1-(5-methyl-1,3,4-oxadiazol-2-yl)piperidin-4-yl)methanone;
((R)-3-(3,5-difluorophenyl)isoxazolidin-2-yl)((3R,4S)-3-fluoro-1-(5-methyl-1,3,4-oxadiazol-2-yl)piperidin-4-yl)methanone;
((S)-3-(3,5-difluorophenyl)isoxazolidin-2-yl)((3S,4R)-3-fluoro-1-(5-methyl-1,3,4-thiadiazol-2-yl)piperidin-4-yl)methanone;
((R)-3-(3,5-difluorophenyl)isoxazolidin-2-yl)((3R,4S)-3-fluoro-1-(5-methyl-1,3,4-thiadiazol-2-yl)piperidin-4-yl)methanone;
((S)-3-(3,5-difluorophenyl)isoxazolidin-2-yl)((3R,4S)-3-fluoro-1-(1,3,4-oxadiazol-2-yl)piperidin-4-yl)methanone;
((R)-3-(3,5-difluorophenyl)isoxazolidin-2-yl)((3S,4R)-3-fluoro-1-(1,3,4-oxadiazol-2-yl)piperidin-4-yl)methanone;
((S)-3-(3,5-difluorophenyl)isoxazolidin-2-yl)((3S,4R)-3-fluoro-1-(1,3,4-oxadiazol-2-yl)piperidin-4-yl)methanone;
((R)-3-(3,5-difluorophenyl)isoxazolidin-2-yl)((3R,4S)-3-fluoro-1-(1,3,4-oxadiazol-2-yl)piperidin-4-yl)methanone;
6-((3R,4S)-4-((S)-3-(3,5-difluorophenyl)isoxazolidine-2-carbonyl)-3-fluoropiperidin-1-yl)pyrimidine-4-carboxamide;
6-((3S,4R)-4-((R)-3-(3,5-difluorophenyl)isoxazolidine-2-carbonyl)-3-fluoropiperidin-1-yl)pyrimidine-4-carboxamide;
6-((3S,4R)-4-((S)-3-(3,5-difluorophenyl)isoxazolidine-2-carbonyl)-3-fluoropiperidin-1-yl)pyrimidine-4-carboxamide;
6-((3R,4S)-4-((R)-3-(3,5-difluorophenyl)isoxazolidine-2-carbonyl)-3-fluoropiperidin-1-yl)pyrimidine-4-carboxamide;
6-((3R,4S)-4-((S)-3-(3,5-difluorophenyl)isoxazolidine-2-carbonyl)-3-fluoropiperidin-1-yl)-N-methylpyrimidine-4-carboxamide;
6-((3S,4R)-4-((R)-3-(3,5-difluorophenyl)isoxazolidine-2-carbonyl)-3-fluoropiperidin-1-yl)-N-methylpyrimidine-4-carboxamide;
6-((3S,4R)-4-((S)-3-(3,5-difluorophenyl)isoxazolidine-2-carbonyl)-3-fluoropiperidin-1-yl)-N-methylpyrimidine-4-carboxamide;
6-((3R,4S)-4-((R)-3-(3,5-difluorophenyl)isoxazolidine-2-carbonyl)-3-fluoropiperidin-1-yl)-N-methylpyrimidine-4-carboxamide;
6-((3R,4S)-4-((S)-3-(3,5-difluorophenyl)isoxazolidine-2-carbonyl)-3-fluoropiperidin-1-yl)-N,N-dimethylpyrimidine-4-carboxamide;
6-((3S,4R)-4-((R)-3-(3,5-difluorophenyl)isoxazolidine-2-carbonyl)-3-fluoropiperidin-1-yl)-N,N-dimethylpyrimidine-4-carboxamide;
6-((3S,4R)-4-((S)-3-(3,5-difluorophenyl)isoxazolidine-2-carbonyl)-3-fluoropiperidin-1-yl)-N,N-dimethylpyrimidine-4-carboxamide;
6-((3R,4S)-4-((R)-3-(3,5-difluorophenyl)isoxazolidine-2-carbonyl)-3-fluoropiperidin-1-yl)-N,N-dimethylpyrimidine-4-carboxamide;
((S)-3-(3,5-difluorophenyl)isoxazolidin-2-yl)((3R,4S)-3-fluoro-1-(5-(methylsulfonyl)pyrimidin-2-yl)piperidin-4-yl)methanone;
((R)-3-(3,5-difluorophenyl)isoxazolidin-2-yl)((3S,4R)-3-fluoro-1-(5-(methylsulfonyl)pyrimidin-2-yl)piperidin-4-yl)methanone;
((S)-3-(3,5-difluorophenyl)isoxazolidin-2-yl)((3S,4R)-3-fluoro-1-(5-(methylsulfonyl)pyrimidin-2-yl)piperidin-4-yl)methanone;
((R)-3-(3,5-difluorophenyl)isoxazolidin-2-yl)((3R,4S)-3-fluoro-1-(5-(methylsulfonyl)pyrimidin-2-yl)piperidin-4-yl)methanone;
((S)-3-(3,5-difluorophenyl)isoxazolidin-2-yl)((3R,4S)-3-fluoro-1-(6-((S)-methylsulfinyl)pyrimidin-4-yl)piperidin-4-yl)methanone;
((R)-3-(3,5-difluorophenyl)isoxazolidin-2-yl)((3S,4R)-3-fluoro-1-(6-((S)-methylsulfinyl)pyrimidin-4-yl)piperidin-4-yl)methanone;
((S)-3-(3,5-difluorophenyl)isoxazolidin-2-yl)((3R,4S)-3-fluoro-1-(6-((R)-methylsulfinyl)pyrimidin-4-yl)piperidin-4-yl)methanone;
((R)-3-(3,5-difluorophenyl)isoxazolidin-2-yl)((3S,4R)-3-fluoro-1-(6-((R)-methylsulfinyl)pyrimidin-4-yl)piperidin-4-yl)methanone;
((S)-3-(3,5-difluorophenyl)isoxazolidin-2-yl)((3S,4R)-3-fluoro-1-(6-((S)-methylsulfinyl)pyrimidin-4-yl)piperidin-4-yl)methanone;
((R)-3-(3,5-difluorophenyl)isoxazolidin-2-yl)((3R,4S)-3-fluoro-1-(6-((S)-methylsulfinyl)pyrimidin-4-yl)piperidin-4-yl)methanone;
((S)-3-(3,5-difluorophenyl)isoxazolidin-2-yl)((3S,4R)-3-fluoro-1-(6-((R)-methylsulfinyl)pyrimidin-4-yl)piperidin-4-yl)methanone;
((R)-3-(3,5-difluorophenyl)isoxazolidin-2-yl)((3R,4S)-3-fluoro-1-(6-((R)-methylsulfinyl)pyrimidin-4-yl)piperidin-4-yl)methanone;
cis-tert-butyl 3-fluoro-4-(3-(2,3,5-trifluorophenyl)isoxazolidine-2-carbonyl) piperidine-1-carboxylate;
trans-tert-butyl 3-fluoro-4-(3-(2,3,5-trifluorophenyl)isoxazolidine-2-carbonyl) piperidine-1-carboxylate;
cis-tert-butyl 3-fluoro-4-(3-(5-fluoropyridin-3-yl)isoxazolidine-2-carbonyl)piperidine-1-carboxylate; and
trans-tert-butyl 3-fluoro-4-(3-(5-fluoropyridin-3-yl)isoxazolidine-2-carbonyl)piperidine-1-carboxylate;
   or pharmaceutically acceptable salts thereof.

It is to be understood that the references herein to a compound of Formula (I) or Formula (II), or a pharmaceutically acceptable salt thereof. Thus, in one embodiment, the invention is directed to a compound of Formula (I) or Formula (II). In another embodiment, the invention is directed to a pharmaceutically acceptable salt of a compound of Formula (I) or Formula (II). In a further embodiment, the invention is directed to a compound of Formula (I) or Formula (II), or a pharmaceutically acceptable salt thereof.

Because of their potential use in medicine, it will be appreciated that a salt of a compound of Formula (I) or Formula (II) is preferably pharmaceutically acceptable. Suitable pharmaceutically acceptable salts can include acid or base addition salts.

As used herein, the term “pharmaceutically acceptable” means a compound which is suitable for pharmaceutical use. Salts and solvates (e.g. hydrates and hydrates of salts) of the compounds of Formulas (I) and (II) which are suitable for use in medicine are those wherein the counterion or associated solvent is pharmaceutically acceptable.

Pharmaceutically acceptable salts include, amongst others, those described in Berge, J. Pharm. Sci., 66, 1-19, (1977) or those listed in P. H. Stahl and C. G. Wermuth, editors, Handbook of Pharmaceutical Salts; Properties, Selection and Use, Second Edition Stahl/Wermuth: Wiley-VCH/VHCA (2011) (see http://www.wiley.com/WileyCDA/WileyTitle/productCd-3906390519.html).

Suitable pharmaceutically acceptable salts can include acid or base addition salts.

Such base addition salts can be formed by reaction of a compound of Formula (I) or Formula (II) (which, for example, contains a carboxylic acid or other acidic functional group) with the appropriate base, optionally in a suitable solvent such as an organic solvent, to give the salt which can be isolated by a variety of methods, including crystallisation and filtration.

Such acid addition salts can be formed by reaction of a compound of Formula (I) or Formula (II) (which, for example contains a basic amine or other basic functional group) with the appropriate acid, optionally in a suitable solvent such as an organic solvent, to give the salt which can be isolated by a variety of methods, including crystallisation and filtration.

Salts may be prepared in situ during the final isolation and purification of a compound of Formula (I) or Formula (II). If a basic compound of Formula (I) or Formula (II) is isolated as a salt, the corresponding free base form of that compound may be prepared by any suitable method known to the art, including treatment of the salt with an inorganic or organic base, suitably an inorganic or organic base having a higher pKa than the free base form of the compound. Similarly, if a compound of Formula (I) or Formula (II) containing a carboxylic acid or other acidic functional group is isolated as a salt, the corresponding free acid form of that compound may be prepared by any suitable method known to the art, including treatment of the salt with an inorganic or organic acid. This invention also provides for the conversion of one salt of a compound of this invention, e.g., a hydrochloride salt, into another salt of a compound of this invention, e.g., a sulfate salt.

It will be understood that if a compound of Formula (I) or Formula (II) contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of Formula (I) or Formula (II) are included within the scope of the invention, including sub-stoichiometric salts, for example where a counterion contains more than one acidic proton.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, tromethamine (tris(hydroxymethyl)aminomethane), and zinc.

It will be understood that if a compound of Formula (I) or Formula (II) contained two or more basic moieties, the stoichiometry of salt formation may include 1, 2 or more equivalents of acid. Such salts would contain 1, 2 or more acid counterions, for example, a diacetate or a dihydrochloride salt.

Because the compounds of Formulas (I) and (II), or a pharmaceutically acceptable salt thereof, are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

The compounds of this invention may be particularly useful for the treatment of RIP1 kinase-mediated diseases or disorders. Such RIP1 kinase-mediated diseases or disorders are diseases or disorders that are mediated by activation of RIP1 kinase, and as such, are diseases or disorders where inhibition of RIP1 kinase would provide benefit.

In this invention, RIP1 kinase-mediated diseases or disorders are diseases or disorders that are mediated by activation of RIP1 kinase, and as such, are diseases or disorders where inhibition of RIP1 kinase would provide benefit. Such RIP1 kinase-mediated diseases or disorders are diseases/disorders which are likely to be regulated at least in part by programmed necrosis, apoptosis or the production of inflammatory cytokines, particularly inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcohol steatohepatitis (NASH), alcohol steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)) Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, ischemic brain injury, traumatic brain injury allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-1 converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic ductal adenocarcinoma, hepatocellular carcinoma, mesothelioma, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, acute liver failure and radiation protection/mitigation, auditory disorders such as noise-induced hearing loss and drugs associated with ototoxicity such as cisplatin, or for the treatment of cells ex vivo to preserve vitality and function.

The compounds of the invention, particularly the compounds of Formula (I) and Formula (II), or a pharmaceutically acceptable salt thereof, may be particularly useful for the treatment of the following RIP1 kinase-mediated diseases or disorders: inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, systemic onset juvenile idiopathic arthritis (SoJIA), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcoholic steatohepatitis (NASH), alcoholic steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)) Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, traumatic brain injury, allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-1 converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, peridontitis, NEMO-mutations (mutations of NF-kappa-B essential modulator gene (also known as IKK gamma or IKKG)), particularly, NEMO-deficiency syndrome, HOIL-1 deficiency ((also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), spinal cord injury, Stevens-Johnson syndrome, fibrosis, complement-mediated cytotoxicity, toxic epidermal necrolysis, and/or for the treatment of cells ex vivo to preserve vitality and function.

The compounds of the invention, particularly the compounds of Formula (I) and Formula (II), or a pharmaceutically acceptable salt thereof, may be particularly useful for the treatment of the following RIP1 kinase-mediated diseases or disorders, that is, diseases/disorders which are likely to be regulated at least in part by RIP1 kinase activity, particularly inflammatory bowel disease (including Crohn's disease and ulcerative colitis), rheumatoid arthritis, chronic obstructive pulmonary disease (COPD), asthma, cigarette smoke-induced damage, cystic fibrosis, psoriasis, retinal detachment, retinal degeneration, retinitis pigmentosa, macular degeneration, atopic dermatitis, burn injury, periodontitis, a bacterial or viral infection (an infection with a pathogen including but not limited to influenza, staphylococcus, and/or mycobacterium (tuberculosis), systemic scleroderma (particularly, topical treatment of hardened and/or tightened skin areas), and/or ischemia reperfusion injury of solid organs/transplant rejection (particularly, topical treatment of donor organ (particularly kidney, liver, and heart and/or lung transplants), infusion of organ recipient), and topical treatment of bowels.

The compounds of the invention, particularly the compounds of Formula (I) and Formula (II), or a pharmaceutically acceptable salt thereof, may be useful for the treatment of glaucoma.

The compounds of the invention, particularly the compounds of Formulas (I) and (II), or a pharmaceutically acceptable salt thereof, may be particularly useful for treatment of pancreatic ductal adenocarcinoma, hepatocellular carcinoma, mesothelioma, or melanoma.

The compounds of the invention, particularly the compounds of Formulas (I) and (II), or a pharmaceutically acceptable salt thereof, may be particularly useful for the treatment of the following RIP1 kinase-mediated disease or disorder: rheumatoid arthritis, inflammatory bowel disease (including Crohn's disease and ulcerative colitis), and psoriasis.

The treatment of the above-noted diseases/disorders may concern, more specifically, the amelioration of organ injury or damage sustained as a result of the noted diseases/disorders. For example, the compounds of this invention may be particularly useful for amelioration of brain tissue injury or damage following ischemic brain injury or traumatic brain injury, or for amelioration of heart tissue injury or damage following myocardial infarction, or for amelioration of brain tissue injury or damage associated with Huntington's disease, Alzheimer's disease or Parkinson's disease, or for amelioration of liver tissue injury or damage associated with non-alcohol steatohepatitis, alcohol steatohepatitis, autoimmune hepatitis autoimmune hepatobiliary diseases, or primary sclerosing cholangitis, or overdose of acetaminophen.

The compounds of this invention may be particularly useful for the amelioration of organ injury or damage sustained as a result of radiation therapy, or amelioration of spinal tissue injury or damage following spinal cord injury or amelioration of liver tissue injury or damage associated acute liver failure. The compounds of this invention may be particularly useful for amelioration of auditory disorders, such as noise-induced hearing loss or auditory disorders following the administration of ototoxic drugs or substances e.g. cisplatin.

The compounds of this invention may be particularly useful for amelioration of solid organ tissue (particularly kidney, liver, and heart and/or lung) injury or damage following transplant or the administration of nephrotoxic drugs or substances e.g. cisplatin. It will be understood that amelioration of such tissue damage may be achieved where possible, by pre-treatment with a compound of Formula (I) or Formula (II), or a pharmaceutically acceptable salt thereof; for example, by pre-treatment of a patient prior to administration of cisplatin or pre-treatment of an organ or the organ recipient prior to transplant surgery. Amelioration of such tissue damage may be achieved by treatment with a compound of Formula (I) or Formula (II), or a pharmaceutically acceptable salt thereof, during transplant surgery. Amelioration of such tissue damage may also be achieved by short-term treatment of a patient with a compound of Formula (I) or Formula (II), or a pharmaceutically acceptable salt thereof, after transplant surgery.

In one embodiment, the compounds of the invention, particularly the compounds of Formulas (I) and (II), or a pharmaceutically acceptable salt thereof, may be useful for the treatment of retinal detachment, macular degeneration, and retinitis pigmentosa.

In another embodiment, the compounds of the invention, particularly the compounds of Formulas (I) and (II), or a pharmaceutically acceptable salt thereof, may be useful for the treatment of multiple sclerosis.

In one embodiment, the compounds of the invention, particularly the compounds of Formulas (I) and (II), or a pharmaceutically acceptable salt thereof, may be useful for the treatment of traumatic brain injury.

In another embodiment, the compounds of the invention, particularly the compounds of Formulas (I) and (II), or a pharmaceutically acceptable salt thereof, may be useful for the treatment of Huntington's Disease, Alzheimer's Disease, amyotrophic lateral sclerosis, and Niemann-Pick disease.

In another embodiment, the compounds of the invention, particularly the compounds of Formula (I) and Formula (II), or a pharmaceutically acceptable salt thereof, may be useful for the treatment of amyotrophic lateral sclerosis (ALS), progressive supranuclear palsy (PSP), and Alzheimer's disease.

In another embodiment, the compounds of the invention, particularly the compounds of Formulas (I) and (II), or a pharmaceutically acceptable salt thereof, may be useful for the treatment of age-related macular degeneration.

The treatment of retinal detachment, macular degeneration, retinitis pigmentosa, multiple sclerosis, traumatic brain injury, Huntington's Disease, Alzheimer's Disease, amyotrophic lateral sclerosis, and Niemann-Pick disease may concern, more specifically, the amelioration of organ injury or damage sustained as a result of these diseases/disorders. For example, the compounds of this invention may be particularly useful for amelioration of brain tissue injury or damage following traumatic brain injury, or for amelioration of brain tissue injury or damage associated of Huntington's Disease, Alzheimer's Disease, amyotrophic lateral sclerosis, and Niemann-Pick disease.

In another embodiment, the compounds of the invention, particularly the compounds of Formulas (I) and (II), or a pharmaceutically acceptable salt thereof, may be useful for the treatment of retinal detachment, macular degeneration, and retinitis pigmentosa, and the amelioration of brain tissue injury or damage as a result of multiple sclerosis, traumatic brain injury, Huntington's Disease, Alzheimer's Disease, amyotrophic lateral sclerosis, and Niemann-Pick disease.

In another embodiment, the compounds of the invention, particularly the compounds of Formulas (I) and (II), or a pharmaceutically acceptable salt thereof, may be useful for the treatment of Crohn's disease, ulcerative colitis, psoriasis, rheumatoid arthritis, spondyloarthritis, systemic onset juvenile idiopathic arthritis (SoJIA), and osteoarthritis.

In yet another embodiment, the compounds of this invention, particularly the compounds of Formula (I) and Formula (II), or a pharmaceutically acceptable salt thereof, may be useful for the treatment of psoriasis, rheumatoid arthritis, and ulcerative and colitis.

In another embodiment, the compounds of this invention, particularly the compounds of Formula (I) and Formula (II), or a pharmaceutically acceptable salt thereof, may be useful for the treatment of lupus, inflammatory bowel disease (IBD), Crohn's disease, and ulcerative colitis.

In another embodiment, the compounds of the invention, particularly the compounds of Formulas (I) and (II), or a pharmaceutically acceptable salt thereof, may be useful for the treatment of cerebrovascular accident (CVA, stroke), Huntington's disease, Alzheimer's disease, amyotrophic lateral sclerosis (ALS), traumatic brain injury, multiple sclerosis, Gaucher disease, Niemann-Pick disease, and spinal cord injury.

In another embodiment, the compounds of the invention, particularly the compounds of Formulas (I) and (II), or a pharmaceutically acceptable salt thereof, may be useful for the treatment of amyotrophic lateral sclerosis (ALS).

In another embodiment, the compounds of the invention, particularly the compounds of Formulas (I) and (II), or a pharmaceutically acceptable salt thereof, may be useful for the treatment of multiple sclerosis.

In another embodiment, the compounds of the invention, particularly the compounds of Formula (I) and Formula (II), or a pharmaceutically acceptable salt thereof, may be useful for the treatment of pancreatic ductal adenocarcinoma (PDAC), metastasis, melanoma, breast cancer, non-small cell lung carcinoma (NSCLC), and radiation induced necrosis.

In another embodiment, the compounds of the invention, particularly the compounds of Formula (I) and Formula (II), or a pharmaceutically acceptable salt thereof, may be useful for the treatment of pancreatic ductal adenocarcinoma (PDAC), metastasis, melanoma, breast cancer, and non-small cell lung carcinoma (NSCLC).

In another embodiment, the compounds of the invention, particularly the compounds of Formula (I) and Formula (II), or a pharmaceutically acceptable salt thereof, may be useful for the treatment of pancreatic ductal adenocarcinoma (PDAC).

In another embodiment, the compounds of the invention, particularly the compounds of Formula (I) and Formula (II), or a pharmaceutically acceptable salt thereof, may be useful for the treatment of intracerebral hemorrhage and subarachnoid hemorrhage.

In another embodiment, the compounds of the invention, particularly the compounds of Formula (I) and Formula (II), or a pharmaceutically acceptable salt thereof, may be useful for the treatment of type II diabetes and obesity.

In another embodiment, the compounds of the invention, particularly the compounds of Formula (I) and Formula (II), or a pharmaceutically acceptable salt thereof, may be useful for the treatment of atherosclerosis.

In another embodiment, the compounds of the invention, particularly the compounds of Formula (I) and Formula (II), or a pharmaceutically acceptable salt thereof, may be useful for the treatment of vasculitis.

In another embodiment, the compounds of the invention, particularly the compounds of Formula (I) and Formula (II), or a pharmaceutically acceptable salt thereof, may be useful for the treatment of burns.

In another embodiment, the compounds of the invention, particularly the compounds of Formula (I) and Formula (II), or pharmaceutically acceptable salt thereof, may be useful for the treatment of ischemic kidney damage, ophthalmologic ischemia, intracerebral hemorrhage, and subarachnoid hemorrhage.

In another embodiment, the compounds of the invention, particularly the compounds of Formula (I) and Formula (II), or pharmaceutically acceptable salt thereof, may be useful f

or the treatment of non-alcoholic steatohepatitis (NASH), alcoholic steatohepatitis (ASH), autoimmune hepatitis, and non-alcoholic fatty liver disease (NAFLD).

The compounds of the invention, particularly the compounds of Formulas (I) and (II), or a pharmaceutically acceptable salt thereof, may be particularly useful for the treatment of the following RIP1 kinase-mediated diseases or disorders. In one aspect the human has a solid tumor. In one aspect the tumor is selected from head and neck cancer, gastric cancer, melanoma, renal cell carcinoma (RCC), esophageal cancer, non-small cell lung carcinoma (NSCLC), prostate cancer, colorectal cancer, ovarian cancer, pancreatic cancer, and pancreatic ductal adenocarcinoma. In one aspect the human has one or more of the following: colorectal cancer (CRC), esophageal cancer, cervical, bladder, breast cancer, head and neck cancer, ovarian cancer, melanoma, renal cell carcinoma (RCC), EC squamous cell carcinoma, non-small cell lung carcinoma, mesothelioma, prostate cancer, and pancreatic ductal adenocarcinoma. In another aspect, the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, astrocytomas, Bannayan-Zonana syndrome, Cowden disease, Lhermitte-Duclos disease, breast cancer, triple negative breast cancer, inflammatory breast cancer, Wilm's tumor, Ewing's sarcoma, Rhabdomyosarcoma, ependymoma, medulloblastoma, colon cancer, head and neck cancer (including squamous cell carcinoma of head and neck), kidney cancer, lung cancer (including lung squamous cell carcinoma, lung adenocarcinoma, lung small cell carcinoma, and non-small cell lung carcinoma), liver cancer (including hepatocellular carcinoma), melanoma, ovarian cancer, pancreatic cancer (including squamous pancreatic cancer), prostate cancer, sarcoma, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, Non-Hodgkin's lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, cancer of the uterus, renal cancer (including kidney clear cell cancer, kidney papillary cancer, renal cell carcinoma), mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

Treatment of RIP1-mediated disease conditions may be achieved using a compound of the invention, particularly a compound of Formula (I) or Formula (II), or a pharmaceutically acceptable salt thereof, of as a monotherapy, or in dual or multiple combination therapy, particularly for the treatment of refractory cases, such as in combination with other anti-inflammatory and/or anti-TNF agents, which may be administered in therapeutically effective amounts as is known in the art.

The compounds of the invention, particularly the compounds of Formula (I) and Formula (II), or a pharmaceutically acceptable salt thereof, may be employed alone or in combination with one or more other therapeutic agents, e.g., pharmaceutically active compounds or biologic products (e.g., monoclonal antibodies). Combination therapies according to the present invention thus comprise the administration of at least one compound of the invention, particularly a compound of Formula (I) or Formula (II), or a pharmaceutically acceptable salt thereof, and at least one other therapeutically active agent. Combination therapies according to the present invention comprise the administration of at least one compound of the invention, particularly a compound of Formula (I) or Formula (II), or a pharmaceutically acceptable salt thereof, and at least one other therapeutic ally active agent, specifically one or two other therapeutically active agents, more specifically one other therapeutically active agent.

For example, amelioration of tissue damage may be achieved by treatment with a compound of Formula (I) or Formula (II), or a pharmaceutically acceptable salt thereof, and at least one other therapeutically active agent during transplant surgery. Amelioration of tissue damage may also be achieved by short-term treatment of a patient with a compound of Formula (I) or (II), or a pharmaceutically acceptable salt thereof, and at least one other therapeutic ally active agent after transplant surgery. Amelioration of tissue damage ex vivo, that is ex vivo preservation of tissues, organs and cells may also be achieved by short-term treatment of tissues, organs and cells with a compound of Formula (I) or Formula (II), or a pharmaceutically acceptable salt thereof, and at least one other therapeutic ally active agent, prior to or during transplant surgery.

The compound(s) of the invention, particularly the compounds of Formula (I) and Formula (II), or pharmaceutically acceptable salts thereof, and the other therapeutic agent(s) may be administered together in a single pharmaceutical composition or separately and, when administered separately this may occur simultaneously or sequentially in any order. The amounts of the compound(s) of the invention, particularly a compound of Formula (I) or Formula (II), or pharmaceutically acceptable salts thereof, and the other therapeutic agent(s) and the relative timings of administration will be selected in order to achieve the desired combined therapeutic effect. Thus, in a further aspect, there is provided a combination comprising a compound of the invention, particularly a compound of Formula (I) or Formula (II), or a pharmaceutically acceptable salt thereof, together with one or more other therapeutic agents, specifically one or two other therapeutically active agents, more specifically one other therapeutically active agent.

Thus, in one aspect of this invention, a compound of the invention, particularly a compound of Formula (I) or Formula (II), or a pharmaceutically acceptable salt thereof, or a pharmaceutical composition comprising a compound of the invention, particularly a compound of Formula (I) or Formula (II), or a pharmaceutically acceptable salt thereof, may be used in combination with or include one or more other therapeutic agents, for example an anti-inflammatory agent and/or an anti-TNF agent.

The pharmaceutical compositions of the invention typically contain one compound of the invention. However, in certain embodiments, the pharmaceutical compositions of the invention contain more than one compound of the invention. In other embodiments, the pharmaceutical compositions of the invention may comprise one or more additional therapeutic agents, specifically one or two other therapeutically active agents, more specifically one other therapeutically active agent.

A compound that inhibits RIP1 kinase, particularly a compound of Formula (I) or Formula (II), or a pharmaceutically acceptable salt thereof, may be administered in combination with other anti-inflammatory agents for any of the indications above, including oral or topical corticosteroids, anti-TNF agents, 5-aminosalicyclic acid and mesalamine preparations, hydroxycloroquine, thiopurines, methotrexate, cyclophosphamide, cyclosporine, calcineurin inhibitors, mycophenolic acid, mTOR inhibitors, JAK inhibitors, Syk inhibitors, anti-inflammatory biologic agents, including anti-IL6 biologics, anti-IL1 agents, anti-IL17 biologics, anti-CD22, anti-integrin agents, anti-IFNa, anti-CD20 or CD4 biologics and other cytokine inhibitors or biologics to T-cell or B-cell receptors or interleukins.

In the treatment of CVA, a compound that inhibits RIP1 kinase, particularly a compound of Formula (I) or Formula (II), or a pharmaceutically acceptable salt thereof, may be administered in combination with antiplatelets (e.g., aspirin, clopidogrel (Plavix®), dipyridamole (Persantine®), ticolpidine (Ticlid®); aspirin and omeprazole (Ysprala®)), anticoagulants (e.g., warfarin (Coumadin®), Heparin®, dabigitran (Pradaxa®), apixaban (Eliquis®), Rivaroxaban®), antihypertensives—diruetics (e.g., Hygroton®, Diuril®, Lasix®, Esidrix®, Hydrodiuril®, Microzide®, Lozol®, Mykrox®, Zaroxolyn®, Midarmar®, Aldactone®, Dyrenium®, Bumex®, Moduretic®, Aldatazide®, Dyazide®, Maxzide®), other antihypertensives—beta blockers, ace inhibitors, angiotensin II receptor blockers, calcium channel blockers, alpha blockers, alpha2 receptor agonist, combined alpha and beta-blockers, central agonists, peripheral adrenergic inhibitors, blood vessel dilators, or tissue plasminogen activator (Alteplase®).

In the treatment of SIRS, a compound that inhibits RIP1 kinase, particularly a compound of Formula (I) or Formula (II), or a pharmaceutically acceptable salt thereof, may be administered in combination with a broad-spectrum antibiotic (such as vacomycin) or other anti-MRSA therapy (cefeprime (Maxipime®), piperacillin/tazobactam (Zosyn®), carbapenem (imipenem, meropenem, doripenem), quinolones (ciprofloxacin, levofloxacin, ofloxacin, moxifloxacin, etc.), or low dose steroids such as hydrocortisones.

In the treatment of inflammatory bowel disease (particularly, Crohn's disease and/or ulcerative colitis), a compound that inhibits RIP1 kinase, particularly a compound of Formula (I) or Formula (II), or a pharmaceutically acceptable salt thereof, may be administered in combination with vedolizumab (Entyvio®), alicaforsen, remestemcel-L (Prochymal®), etrolizumab, eldelumab, or bertilimumab.

In the treatment of psoriasis, a compound that inhibits RIP1 kinase, particularly a compound of Formula (I) or Formula (II), or a pharmaceutically acceptable salt thereof, may be administered in combination with ixekizumab, tildrakizumab (MK-3222), secukinumab (AIN457), Alefacept (Amevive®), calcipotriene and betamethasone dipropionate (Enstilar®), prednisone (Rayos®), tazorac topical gel, Methotrexate (Trexall®, Rheumatrex®, Folex PFS®, Otrexup®, Rasuvo®, Methotrexate LPF Sodium®), Cyclosporine®, fumaric acid, Acitretin®, Tretinate®, UVA, UVB, Psoralen, coal tar, TNF inhibitors (Etanercept (Enbrel®), Infliximab (Remicade®), adalimumab (Humira®); certolizumab pegol (Cimzia®)), PDE-4 inhibitors (apremilast (Otezla®)), JAK inhibitors (Tofacitinib (Xeljanz® CP-690550), IL 12/23 (ustekinumab (Stelara®)), IL17 (secukinumab (Coxentyx®), ixekizumab (Taltz®), brodalumab with AMG-827), IL23 (tildrakizumab with MK-3222, guselkumab CNTO-1959, BI 655066, itolizumab (Alzumab®), biosimilars infliximab (Remsima (Inflectra®), Sandoz GP 11111), biosimilars rituximab (CT-P10 (Mabthera®), PF-05280586 (MabThera®)), biosimilars etanercept (CHS-2014), biosimilars adalimumab (GP-2017), M-518101 topical vitamin D; Maruho GK-664, or CT-327 (topical Tropomyosin-receptor kinase A), CF-101, or dimethyl fumarate LAS-41008.

In the treatment of periodonitis, a compound that inhibits RIP1 kinase, particularly a compound of Formula (I) or Formula (II), or a pharmaceutically acceptable salt thereof, may be administered in combination with an antimicrobial agent, (such as chlorhexidine (Peridex®, PerioChip®, PerioGard®, etc.)) or an antibiotic (such as doxycycline (Vibrox®, Periostat®, Monodox®, Oracea®, Doryx®, etc.), or minocycline (Dynacin®, Minocin®, Arestin®, Dynacin®, etc.).

In the treatment of asthma, a compound that inhibits RIP1 kinase, particularly a compound of Formula (I) or Formula (II), or a pharmaceutically acceptable salt thereof, may be administered in combination with an inhaled corticosteroid (ICS) such as fluticasone proprionate (Flovent®), fluticasone furoate (Veramyst®/Avamys®), beclomethasone dipropionate (QVAR®), budesonide (Pulmicort), trimcinolone acetonide (Azmacort®), flunisolide (Aerobid®), mometasone fuorate (Asmanex® Twisthaler®), or Ciclesonide (Alvesco®), a long acting beta agonist (LABA) such as formoterol fumarate (Foradil®), salmeterol xinafoate (Serevent®), indacaterol (Arcapta® Neohaler®); a combination of an ICS and LABA (such as fluticasone furoate and vilanterol (Breo Ellipta®/Relvar Ellipta®), formoterol/budesonide inhalation (Symbicort®), mometasone furoate/formoterol fumarate dihydrate (Dulera®), beclomethasone dipropionate/formoterol (Inuvair®), fluticasone propionate/eformoterol fumarate dehydrate (Flutiform®), and fluticasone propionate/salmeterol (Advair®), a short acting beta agonist ((SABA) such as salbutamol dry-powder inhalation, albuterol sulfate (ProAir®, Proventil HFA®, Ventolin HFA®, AccuNeb® Inhalation Solution), levalbuterol tartrate (Xopenex® HFA), an antimuscarinic agent such as ipratropium bromide (Atrovent® HFA); an antimuscarinic in combination with a beta-agonist such as ipratropium bromide/albuterol (Combivent® Respimat®); a long-acting muscarinic antagonist ((LAMA) such as umeclidinium bromide (Incruse®) ortiotropium bromide (Spiriva® HandiHaler; a combination of a LAMA and a LABA, such as umeclidinium bromide and vilanterol (Anoro®) a leukotriene modifier (such as montelukast sodium (Singulair®), zafirlukast (Accolate®), or zileuton (Zyflo®), and anti-IgE (such as omalizumab (Xolair®)), a methylxanthine bronchodilator (such as theophylline (Accurbron®, Aerolate®, Aquaphyllin®, Asbron®, Bronkodyl®, Duraphyl®, Elixicon®, Elixomin®, Elixophyllin®, Labid®, Lanophyllin®, Quibron-T®, Slo-Bid®, Slo-Phyllin®, Somophyllin®, Sustaire®, Synophylate®, T-Phyll®, Theo-24®, Theo-Dur®, Theobid®, Theochron®, Theoclear®, Theolair®, Theolixir®, Theophyl®, Theovent®, Uni-dur®, Uniphyl®), a mast cell inhibitor (such as cromulyn sodium (Nasalcrom®) and nedocromil sodium (Tilade®)).

Other agents that may be suitable for use in combination therapy in the treatment of asthma include a protein tyrosine kinase inhibitor (masitinib), CRTH2/D-prostanoid receptor antangonist (AMG 853), an epinephrine inhalation aerosol (E004), reslizumab, Vectura's VR506, lebrikizumab (RG3637), a combination phosphodiesterase (PDE)-3 and (PDE)-4 inhibitor (RPL554).

In the treatment of COPD, a compound that inhibits RIP1 kinase, particularly a compound of Formula (I) or Formula (II), or a pharmaceutically acceptable salt thereof, may be administered in combination with a LABA (such as salmeterol xinafoate (Serevent), aformoterol tartrate (Brovana®), formoterol fumarate inhalation powder (Foradil®), indacterol maleate (Arcapta® Neohaler®), a long-acting inhaled anticholinergic (or muscannic antagonist, such as umeclidinium (Incruse Ellipta®), tiotropium bromide (Spiriva®), and aclidinium bromide (Tudorza® Pressair®), a phosphodiesterase (PDE-r) inhibitor (such as roflumilast, Daliresp®), a combination ICS/LABA (such as fluticasone furoate and vilanterol (Breo Ellipta®/Relvar Ellipta®), fluticasone propionate/salmeterol (Advair®), budesonide/formoterol (Symbicort®), mometasone/formoterol (Dulera®), or fluticasone propionate/eformoterol fumarate dehydrate (Flutiform®); an antimuscannic such as such as ipratropium bromide (Atrovent®); an antimuscarinic in combination with a beta-agonist such as ipratropium bromide/albuterol (Combivent® Respimat®); a long-acting antimuscarinic such as umeclidinium bromide (Incruse®) or tiotropium bromide (Spiriva®); umeclidinium/vilanterol (Anoro Ellipta®); a combination of a LAMA and a LABA, such as umeclidinium bromide and vilanterol (Anoro®).

Other agents that may be suitable for use in combination therapy in the treatment of COPD include SCH527123 (a CXCR2 antagonist), glycoprronium bromide ((NVA237) Seebri® Breezhaler®), glycopyrronium bromide and indacaterol maleate ((QVA149) Ultibro® Breezhaler®), glycopyrrolate and formoterol fumarate (PT003), indacaterol maleate (QVA149), olodaterol (Striverdi® Respimat®), tiotropium (Spiriva®)/olodaterol (Striverdi® Respimat®), and aclidinium/formoterol inhalation.

In the treatment of a mycobacterium infection (tuberculosis), a compound that inhibits RIP1 kinase, particularly a compound of Formula (I) or Formula (II), or a pharmaceutically acceptable salt thereof, may be administered in combination with an antimycobacterial agent (such as isoniazid (INH), ehambutol (Myambutol®), rifampin (Rifadin®), and pyrazinamide (PZA)) a bactericidal antibiotic (such as rifabutin (Mycobutin®) or rifapentine (Priftin®)), an aminoglycoside (Capreomycin®), a fluorquinolone (levofloxacin, moxifloxicin, ofloxacin), thioamide (ehionamide), cyclosporine (Sandimmune®), para-aminosalicyclic acid (Paser®), cycloserine (Seromycin®), kanamycin (Kantrex®), streptomycin, viomycin, capreomycin (Capastat®)), bedaquiline fumarate (Sirturo®), oxazolidinone (Sutezolid®), or delamanid (OPC-67683).

In the treatment of systemic scleroderma, a compound that inhibits RIP1 kinase, particularly a compound of Formula (I) or Formula (II), or a pharmaceutically acceptable salt thereof, may be administered in combination with an oral corticosteroid (such as prednisolone (Delatsone®, Orapred, Millipred, Omnipred, Econopred, Flo-Pred), an immunosuppressive agent (such as methotrexate (Rhuematrex®, Trexall®), cyclosporine (Sandimmune®), anti-thymocyte globulin (Atgam®), mycophenolate mofetil (CellCept®), cyclophosphamide (Cytoxan®), FK506 (tacrolimus), thalidomide (Thalomid®), chlorambucil (Leukeran®), azathioprine (Imuran®, Azasan®)), a calcium channel blocker (such as nifedipine (Procardia®, Adalat®) or nicardipine (Cardene®), a topical emollient (nitroglycerin ointment), an ACE inhibitor (such as lisinopril (Zestril®, Prinivil®), diltaizem (Cardizem®, Cardizem SR®, Cardizem CD®, Cardia®, Dilacor®, Tiazac®)), a serotonin reuptake inhibitor (such as fluoxetine (Prozac®)), an endothelin-1 receptor inhibitor (such as bosentan (Tracleer®) or epoprostenol (Flolan®, Veletri®, Prostacyclin®)) an anti-fibrotic agent (such as colchicines (Colcrys®), para-aminobenzoic acid (PABA), dimethyl sulfoxide (KMSO), and D-penicillamine (Cuprimine®, Depen®), interferon alpha and interferon gamma (INF-g)), a proton-pump Inhibitor (such as omeprazole (Prilosec®), metoclopramide (Reglan®), lansoprazole (Prevacid®), esomeprazole (Nexium®), pantoprazole (Protonix®), rabeprazole (Aciphex®)) or imatinib (Gleevec®) ARG201 (arGentis Pharmaceutical), belimumab (Benlysta®), tocilizumab (Actema®).

In the treatment of cystic fibrosis, a compound that inhibits RIP1 kinase, particularly a compound of Formula (I) or Formula (II), or a pharmaceutically acceptable salt thereof, may be administered in combination with a cystic fibrosis transmembrane conductance regulator (CFTR) potentiator (ivacftor (Kalydeco®)) a mucolytic agent (such as dornase alpha (Pulmozyme®)), pancreatic enzymes (such as Pancrelipase (Creon®, Pancreaze®, Ultresa®, Zenpep®)), a bronchodilator (such as albuterol (AccuNeb®, ProAir®, Proventil HFA®, VoSpire ER®, Ventolin HFA®)), an antibiotic (including inhaled, oral or parenteral, such as tobramycin solution for inhalation (TOBI®, Bethkis®, TOBI Podhaler®), aztreonam inhalation (Azactam®, Cayston®), colistimethate sodium (Coly-Mycin®), cephalosporins (cefadroxil monohydrate (Duricef®), cefazolin (Kefzol®), cephalexin (Keflex®), cefazolin (Ancef®, etc.), fluoroquinolones (moxifloxacin, levofloxacin, gemifloxacin, etc.), azithromycin (Zithromax®), gentamicin (Garamycin®), piperacillin/tazobacam (Zosyn®), cephalexin (Keflex), ceftazidime (Fortaz, Tazicef), ciprofloxin (Cipro XR, Proquin XR), trimethoprim/sulfamethoxazolyl (Bactrim DS, Septra DS), chloramphenicol)), or ivacftor (Kalydeco®)/lumacaftor (VX-809), ataluren (Translarna®), or with tiopropium bromide (Spiriva® Handihaler®) as add on to standard therapy.

In the treatment of retinitis pigmentosa, a compound that inhibits RIP1 kinase, particularly a compound of Formula (I) or Formula (II), or a pharmaceutically acceptable salt thereof, may be administered in combination with a ciliary neurtotrophic growth factor (NT-501-CNTF) or gene transfer agent, UshStat®.

In the treatment of macular degeneration, a compound that inhibits RIP1 kinase, particularly a compound of Formula (I) or Formula (II), or a pharmaceutically acceptable salt thereof, may be administered in combination with opthalmalic intravitreal injections (afibercept (Eylea®)) or with an anti-vascular endothelial growth factor (VEGF) inhibitor (such as ranibizumab (Lucentis®) or pegaptanib sodium (Macugen®)), a ciliary neurotrophic growth factor agent (NT501), iSONEP®, or bevacizumab (Avastin®).

In the treatment of influenza, a compound that inhibits RIP1 kinase, particularly a compound of Formula (I) or Formula (II), or a pharmaceutically acceptable salt thereof, may be administered in combination with a trivalent (IIV3) inactivated influenza vaccine (such as Afluria®, Fluarix®, Flucelvax®, FluLaval®, Fluvirin®, Fluzone®), a quadrivalent (IIV4) inactivated influenza vaccine (such as Fluarix® Quadrivalent, Flulaval® Quadrivalent, Fluzone® Quadrivalent), a trivalent recombinant influenza vaccine (such as FluBlok®), a quadrivalent live attenuated influenza vaccine (such as FluMist® Quadrivalent), an antiviral agent (such as oseltamivir (Tamiflu®), zanamivir (Relenza®), rimantadine (Flumadine®), or amantadine (Symmetrel®)), or Fluad®, Fludase, FluNhance®, Preflucel, or VaxiGrip®.

In the treatment of a staphylococcus infection, a compound that inhibits RIP1 kinase, particularly a compound of Formula (I) or Formula (II), or a pharmaceutically acceptable salt thereof, may be administered in combination with an antibiotic (such as a β-Lactam cephalosporin (Duricef®, Kefzol®, Ancef®, Biocef®, etc.), nafcillin (Unipen®), a sulfonamide (sulfamethoxazolyl and trimethoprim (Bacrim®, Septra®,) sulfasalazine (Azulfidine®), acetyl sulfisoxazolyl (Gantrisin®, etc.), or vancomycin (Vancocin®)).

In the treatment of transplant rejection, a compound that inhibits RIP1 kinase, particularly a compound of Formula (I) or Formula (II), or a pharmaceutically acceptable salt thereof, may be administered in combination with a high-dose corticosteroid (such as prednisone (Deltasone®), methylprednisolone (SoluMedrol®) etc.) a calcineurin inhibitor (such as cyclosporine (Sandimmune®, Neoral®, Gengraf®), tacrolimus (Prograf®, Astragraf XL®)), an mTor inhibitor (such as sirolimus (Rapamune®) or everolimus (Afinitor®)), an anti-proliferative agent (such as azathioprine (Imuran®, Azasan®), mycophenolate mofetil (CellCept®), or mycophenolate sodium (Myfortic®)), a monoclonal antibody (such as muromonab-CD3 (Orthoclone OKT3®)), an interleukine-2 receptor antagonist ((Basiliximab®, Simulect®), daclizumab (Zenapax®), or rituximab (Rituxan®)), a polyclonal anti-T-cell antibody (such as anti-thymocyte gamma globulin-equine (Atgam®), or antithymocyte globulin-rabbit (Thymoglobulin®)) an anti-CD40 antagonist (ASKP-1240), a JAK inhibitor (ASP015K), or an anti-TCR murine mAb (TOL101).

In the treatment of atopic dermatitis, a compound that inhibits RIP1 kinase, particularly a compound of Formula (I) or Formula (II), or a pharmaceutically acceptable salt thereof, may be administered in combination with a topical immunomodulator or calcineurin inhibitor (such as pimecrolimus (Elidel®) or tacrolimus ointment (Protopic®)), a topical corticosteroid (such as hydrocortizone (Synacort®, Westcort®), betamethasone (Diprolene®), flurandrenolide (Cordan®), fluticasone (Cutivate®), triamcinolone (Kenalog®), fluocinonide (Lidex®), and clobetasol (Temovate®)), an oral corticosteroid (such as hydrocortisone (Cortef®), methylprednisolone (Medrol®), or prednisolone (Pediapred®, Prelone®), an immunosuppressant (such as cyclosporine (Neoral®) or interferon gamma (Alferon N®, Infergen®, Intron A, Roferon-A®)), an antihistamine (for itching such as Atarax®, Vistaril®, Benadryl®), an antibiotic (such as penicillin derivatives flucloxacillin (Floxapen®) or dicloxacillin (Dynapen®), erythromycin (Eryc®, T-Stat®, Erythra-Derm®, etc.)), anon-steroidal immunosuppressive agent (such as azathioprine (Imuran®, Azasan®), methotrexate (Rhuematrex®, Trexall®), cyclosporin (Sandimmune®), or mycophenolate mofetil (CellCept®)).

In the treatment of spondyloarthritis, a compound that inhibits RIP 1 kinase, particularly a compound of Formula (I) or Formula (II), or a pharmaceutically acceptable salt thereof, may be administered in combination with NSAIDs, DMARDs such as Sulfasalazine®, Methotrexate®, and corticosteroids; prednisolone delayed-release tablets (Rayos®), TNF inhibitors (Enbrel®, Remicade®, Humira® and Simponi®), or IL-17A (Cosentyx®).

In the treatment of systemic onset juvenile idiopathic arthritis (sJIA), a compound that inhibits RIP 1 kinase, particularly a compound of Formula (I) or Formula (II), or a pharmaceutically acceptable salt thereof, may be administered in combination with NSAIDs such as Celebrex®, diclofenac (Voltaran®), ibuprofen (Advil®, Motrin®), naproxen (Aleve, Naprosyn®), corticosteroids (prednisone, glucocorticoids), Methotrexate®, or biologics (ankinra (Kineret®), tocilizumab (Actemra®), canakinumab (ILARIS®)).

In the treatment of osteoarthritis, a compound that inhibits RIP 1 kinase, particularly a compound of Formula (I) or Formula (II), or a pharmaceutically acceptable salt thereof, may be administered in combination with analgesics and NSAIDs (acetaminophen, opioid narcotics (e.g., Tramadol®, Vicodin®, Darvon®, Percocet®); ibuprofen and famotidine (Duexis®); Etadolac®; naproxen sodium (Naprelan®), diclofenac sodium topical solution (Pennsaid®); sodium hyaluronate (Supartz®); meloxicam (Vivlodex®, Mobic®); acetaminophen, ibuprofen, aspirin, Celecoxib®, COX-2 (Celebrex®), valdecoxib (Bextra®)), corticosteroid injections, hyaluronic acid injection (Gelsyn-3®); hylan GF 20 (Synvisc, @Synvisc-One®), or duloxetine hydrochloride (Cymbalta®).

In the treatment of or medication management of Huntington's disease, a compound that inhibits RIP 1 kinase, particularly a compound of Formula (I) or Formula (II), or a pharmaceutically acceptable salt thereof, may be administered in combination with tetrabenazine (Xenazine®), antipsychotic drugs (haloperidol (Haldol®), chlorpromazine HCL (Thorazine®), risperidone (Risperdal®) and quetiapine (Seroquel®)), drugs to suppress chorea (amantadine, devetiracetam (Keppra®), clonazepam (Klonopin®)), antidepressants (citalopram (Celexa®, Lexapro®), fluoxetine (Prozac®, Sarafem®), sertraline (Zoloft®)), antipsychotics (quetiapine (Seroquel®), risperidone (Risperdal®), olanzapine (Zyprexa®)), or mood-stabilizing drugs (vaproate (Depacon®), carbamazepine (Carbatrol®, Epitol®, Equetro®), lamotrigine (Lamictal®)).

In the treatment of Alzheimer's, a compound that inhibits RIP 1 kinase, particularly a compound of Formula (I) or Formula (II), or a pharmaceutically acceptable salt thereof, may be administered in combination with Donepzil hydrocholoride (Aricept®), Rivastigmine tartrate (Exelon®), caprylidene (Axona®), butoconazole nitrate 2% (Femstat 3®), Galantamine hydrobromide (Razadyne®, Reminyl®), Memantine HCL (Namenda@), memantine hydrocholoride extended release+donepezil hydrochloride (Namzaric®), Solanezumab, beta-secretase with Merck (MK-8931), beta-secretase with Cerespir (CSP-1103), or drugs that targets tau protein (AADvac1).

In the treatment of Amyotrophic lateral sclerosis (ALS), a compound that inhibits RIP 1 kinase, particularly a compound of Formula (I) or Formula (II), or a pharmaceutically acceptable salt thereof, may be administered in combination with a glutamate blocker (Riluzole (Rilutek®)), In the treatment of symptoms with ALS, a compound that inhibits RIP 1 kinase, particularly a compound of Formula (I) or Formula (II), or a pharmaceutically acceptable salt thereof, may be administered in combination with quinidine (Nuedexta®), anticholinergics (Amitriptyline®, Artane®, scopolamine patch (Transderm Scop®)), sympathomimetics (pseudoephedrine), mucolytics (guaifenesin), or analgesics (tramadol (Ultram®); ketorolac (Toradol®); morphine; fentanyl patch (Duragesic®)).

In the treatment of multiple sclerosis, a compound that inhibits RIP 1 kinase, particularly a compound of Formula (I) or Formula (II), or a pharmaceutically acceptable salt thereof, may be administered in combination with corticosteroids (prednisone, methylprednisolone), Interferon Beta 1-A (Avonex®, Extavia®, Rebif®, Betaseron®), peginterferon beta-1A (Plegridy®), Glatiramer acetate (Copaxone®); glatiramer acetate (Glatopa®—generic equivalent of Copaxone); Dimethyl fumarate (Tecfidera®); Fingolimod (Gilenya®); teriflunomide (Aubagio®); dalfampridine (Ampyra®); daclizumab (Zinbryta); alemtuzumab (Lemtrada®); natalizumab (Tysabri®); or mitoxantrone hydrochloride (Novantrone®).

In the treatment of gaucher disease, a compound that inhibits RIP 1 kinase, particularly a compound of Formula (I) or Formula (II), or a pharmaceutically acceptable salt thereof, may be administered in combination with enzyme replacement therapy (imiglucerase (Cerezyme®), velaglucerase alfa (VPRIV®), taliglucerase alfa (Elelyso®)) or substrate reduction therapy (miglustat (Zavesca®), eliglustat (Cerdelga®)).

In the treatment of Niemann-Pick Disease, a compound that inhibits RIP 1 kinase, particularly a compound of Formula (I) or Formula (II), or a pharmaceutically acceptable salt thereof, may be administered in combination with bone marrow transplant, enzyme replacement therapy, gene therapy, miglustat (Zavesca®), Arimoclomol (BRX-345), NCT02612129, Hydroxypropyl-beta-cyclodexin (HPbCD), NCT01747135, or Hydroxypropyl-β-cyclodextrin (VTS-2702) (NCT02534844).

In the treatment of rheumatoid arthritis, a compound that inhibits RIP 1 kinase, particularly a compound of Formula (I) or Formula (II), or a pharmaceutically acceptable salt thereof, may be administered in combination with Tocilizumab (Actemra®), Arava, sulfasalazine delayed release tablets (Azulfidine EN-tabs®, Bextra, certolizumab pegol (Cimzia®), ibuprofen and famotidine (Duexis®), naproxen sodium (Etodolac®), adalimumab (Humira®), Kineret; etodolac (Lodine®), naproxen sodium (Naprelan), abatacept (Orencia), prednisone (Rayos®), inflimimab (Remicade®), golimuma (Simponi®), rofecoxib (Vioxx®), tofacitinib (Xeljanz®), methotrexate (Trexall®, Rheumatrex®, Folex PFS®, Otrexup®, Rasuvo®, Methotrexate LPF Sodium®, selective JAK1 & JAK2 inhbitor (baracitinib), antisense oligonucleotide (alicafosen), biosimilars for infliximab (Remsima (Inflectra®)), GS-071 infliximab (Aprogen), SB2 infliximab, PF-06438179 infliximab, GP 11111, biosimilars for rituximab (CT-P10 rituximab Celltrion), BI-695500, GP-2013, PF-05280586, biosimilars for etanercept (etanercept SB4 (Brenzys™), Benepali®; CHS-0214 etanercept, GP-2015, biosimilars for adalimumab (ABP-501 adalimumab, BI-695501, Samsung SB5, GP-2017. PF-06410293, Momenta M923, or biosimilar for abatacept (M834).

In the treatment of ulcerative colitis, a compound that inhibits RIP 1 kinase, particularly a compound of Formula (I) or Formula (II), or a pharmaceutically acceptable salt thereof, may be administered in combination with alicafosen, Mesalamine (Asacol®), balsalazide disodium (Colazal®), vedolizumab (Entyvio®), golimumab (Simponi®), budesonide (Uceris®), adalimumab (Humira®), RG-7413 (alpha4beta7 integrin), CNTO-1275 (ustekinumab), biosimiar infliximab (Remsima (Inflectra®)), BMS eldelumab (CXCL 10), or Immune Pharma bertilimumab (CCR3).

In the treatment of Crohn's disease, a compound that inhibits RIP 1 kinase, particularly a compound of Formula (I) or Formula (II), or a pharmaceutically acceptable salt thereof, may be administered in combination with Remestemcel-L (Prochymal®), vedolizumab (Entyvio®), ustekinumab (Stelara®), certolizumab pegol (Cimzia®), natalizumab (Tysabri®), budesonide (Entocort EC®), anti-inflammatories (mesalamine (Lialda®, Apriso®, Canasa®, Asacol®, Rowasa®), sulfasalazine (Azulfidine®)), steroids (hydrocortisone, prednisone), immunosuppressants (methotrexate (Trexall®, Rasuvo®, Rheumatrex®), infliximab (Remicade®), azathioprine (Azasan®, Imuran®), adalimumab (Humira®), mercaptopurine (Purinethol®, Purixan®); cyclosporine (Gengraf®, Neoral®, Sandimuune®); tacrolimus (Astagraf XL®, Hecoria®)), or antibiotics (metronidazole (Flagyl®, Metrogel®, Noritate®, MetroCream®, Rosadan®, MetroLotion®); ciprofloxacin (Cipro®)).

In one embodiment of this invention, the at least one other therapeutically active agent is selected from a thrombolytic agent, a tissue plasminogen activator, an anticoagulant, and a platelet aggregation inhibitor. In another embodiment, the at least one other therapeutically active agent is selected from heparin, coumadin, clopidrogel, dipyridamole, ticlopidine HCL, eptifibatide, and aspirin. In one embodiment, the RIP1 kinase-mediated disease or disorder treated with these agents is a cerebrovascular accident.

In one embodiment of this invention, the at least one other therapeutically active agent is selected from broad-spectrum antibiotic, anti-MRSA therapy and a low dose steroid. In another embodiment, the at least one other therapeutically active agent is selected from vacomycin, cefeprime, a combination of piperacillin and tazobactam, imipenem, meropenem, doripenem, ciprofloxacin, levofloxacin, ofloxacin, moxifloxacin, and hydrocortisone. In one embodiment, the RIP1 kinase-mediated disease or disorder treated with these agents is systemic inflammatory response syndrome.

In one embodiment of this invention, the at least one other therapeutically active agent is alicaforse or remestemcel-L. In one embodiment, the RIP1 kinase-mediated disease or disorder treated with these agents is Crohn's disease or ulcerative colitis.

In one embodiment of this invention, the at least one other therapeutically active agent is ixekizumab, or tildrakizumab. In one embodiment, the RIP1 kinase-mediated disease or disorder treated with these agents is psoriasis.

In one embodiment of this invention, the at least one other therapeutically active agent is an antimicrobial agent or an antibiotic. In another embodiment, the at least one other therapeutically active agent is selected from chlorhexidine, doxycycline and minocycline. In one embodiment, the RIP1 kinase-mediated disease or disorder treated with these agents is periodonitis.

In one embodiment of this invention, the at least one other therapeutically active agent is selected from an inhaled corticosteroid, a long acting beta agonist, a combination of an inhaled corticosteroid and a long acting beta agonist, a short acting beta agonist, a leukotriene modifier, an anti-IgE, a methylxanthine bronchodilator, a mast cell inhibitor, and a long-acting muscarinic antagonist. In another embodiment, the at least one other therapeutically active agent is selected from fluticasone proprionate, beclomethasone dipropionate, budesonide, trimcinolone acetonide, flunisolide, mometasone fuorate, or ciclesonide, formoterol fumarate, salmeterol xinafoate, a combination of fluticasone furoate and vilanterol, a combination of formoterol and budesonide inhalation, a combination of beclomethasone dipropionate and formoterol, a combination of fluticasone propionate and salmeterol, albuterol sulfate, levalbuterol tartrate, a combination of ipratropium bromide and albuterol, ipratropium bromide, montelukast sodium, zafirlukast, zileuton, omalizumab theophylline, cromulyn sodium, nedocromil sodium, and a combination of mometasone furoate and formoterol fumarate dihydrate. In another embodiment, the at least one other therapeutically active agent is selected from protein tyrosine kinase inhibitor, a CRTH2/D-prostanoid receptor antangonist, an epinephrine inhalation aerosol, and a combination of a phosphodiesterase-3 inhibitor and a phosphodiesterase-4 inhibitor. In another embodiment, the at least one other therapeutically active agent is selected from masitinib, AMG 853, indacaterol, E004, a combination of fluticasone furoate and fluticasone proprionate, a combination of vinanterol fluticasone furoate, a combination of fluticasone propionate and eformoterol fumarate dehydrate, reslizumab, salbutamol, tiotropium bromide, a combination of formoterol and budesonide, fluticasone furoate, VR506, lebrikizumab, and RPL554. In one embodiment, the RIP1 kinase-mediated disease or disorder treated with these agents is asthma.

In one embodiment of this invention, the at least one other therapeutically active agent is selected from a long acting beta agonist, a long-acting inhaled anticholinergic or muscarinic antagonist, a phosphodiesterase inhibitor, a combination an inhaled corticosteroid long acting beta agonist, a short acting beta agonist, and an inhaled corticosteroid. In another embodiment, the at least one other therapeutically active agent is selected from salmeterol xinafoate, a combination of umeclidinium and vilanterol, umeclidinium, aformoterol tartrate, formoterol fumarate, indacterol maleate, a combination of fluticasone propionate and eformoterol fumarate dehydrate, tiotropium bromide, aclidinium bromide, roflumilast, a combination of fluticasone furoate and vilanterol, a combination of fluticasone propionate and salmeterol, a combination of budesonide and formoterol, a combination of mometasone and formoterol, a combination of ipratropium bromide and albuterol sulfate, a combination of albuterol and ipratropium, ipratropium bromide, albuterol sulfate, budesonide, fluticasone propionate, and beclometasone dipropionate. In another embodiment, the at least one other therapeutically active agent is selected from SCH527123, glycoprronium bromide, a combination of glycopyrronium bromide and indacaterol maleate, a combination of glycopyrrolate and formoterol fumarate, indacaterol maleate, olodaterol, tiotropium, olodaterol, and a combination of aclidinium and formoterol. In one embodiment, the RIP1 kinase-mediated disease or disorder treated with these agents is COPD.

In one embodiment of this invention, the at least one other therapeutically active agent is an antimycobacterial agent or a bactericidal antibiotic. In another embodiment, the at least one other therapeutically active agent is selected from isoniazid, ehambutol, rifampin, pyrazinamide, rifabutin, rifapentine, capreomycin, levofloxacin, moxifloxicin, ofloxacin, ehionamide, cycloserine, kanamycin, streptomycin, viomycin, bedaquiline fumarate, PNU-100480, and delamanid. In one embodiment, the RIP1 kinase-mediated disease or disorder treated with these agents is a mycobacterium infection.

In one embodiment of this invention, the at least one other therapeutically active agent is selected from an oral corticosteroid, anti-thymocyte globulin, thalidomide, chlorambucil, a calcium channel blocker, a topical emollient, an ACE inhibitor, a serotonin reuptake inhibitor, an endothelin-1 receptor inhibitor, an anti-fibrotic agent, a proton-pump inhibitor or imatinib, ARG201, and tocilizumab. In another embodiment, the at least one other therapeutically active agent is selected from prednisolone, anti-thymocyte globulin, FK506 (tacrolimus), thalidomide, chlorambucil, nifedipine, nicardipine, nitroglycerin ointment, lisinopril, diltaizem, fluoxetine, bosentan, epoprostenol, colchicines, para-aminobenzoic acid, dimethyl sulfoxide, D-penicillamine, interferon alpha, interferon gamma (INF-g)), omeprazole, metoclopramide, lansoprazole, esomeprazole, pantoprazole, rabeprazole, imatinib, ARG201, and tocilizumab. In one embodiment, the RIP1 kinase-mediated disease or disorder treated with these agents is systemic scleroderma.

In one embodiment of this invention, the at least one other therapeutically active agent is selected from a cystic fibrosis transmembrane conductance regulator potentiator, a mucolytic agent, pancreatic enzymes, a bronchodilator, an antibiotic, or ivacftor/lumacaftor, ataluren, and tiopropium bromide. In another embodiment, the at least one other therapeutically active agent is selected from ivacftor, dornase alpha, pancrelipase, albuterol, tobramycin, aztreonam, colistimethate sodium, cefadroxil monohydrate, cefazolin, cephalexin, cefazolin, moxifloxacin, levofloxacin, gemifloxacin, azithromycin, gentamicin, piperacillin/tazobacam, ceftazidime, ciprofloxin, trimethoprim/sulfamethoxazolyl, chloramphenicol, or ivacftor/lumacaftor, ataluren, and tiopropium bromide. In one embodiment, the RIP1 kinase-mediated disease or disorder treated with these agents is cystic fibrosis.

In one embodiment of this invention, the at least one other therapeutically active agent is a ciliary neurtotrophic growth factor or a gene transfer agent. In another embodiment, the at least one other therapeutically active agent is NT-501-CNTF or a gene transfer agent encoding myosin VIIA (MY07A). In one embodiment, the RIP1 kinase-mediated disease or disorder treated with these agents is retinitis pigmentosa.

In one embodiment of this invention, the at least one other therapeutically active agent is selected from opthalmalic intravitreal injections, an anti-vascular endothelial growth factor inhibitor, and a ciliary neurotrophic growth factor agent. In another embodiment, the at least one other therapeutically active agent is selected from afibercept, ranibizumab, pegaptanib sodium, NT501, humanized sphingomab, and bevacizumab. In one embodiment, the RIP1 kinase-mediated disease or disorder treated with these agents is macular degeneration.

In one embodiment of this invention, the at least one other therapeutically active agent is selected from a trivalent (IIV3) inactivated influenza vaccine, a quadrivalent (IIV4) inactivated influenza vaccine, a trivalent recombinant influenza vaccine, a quadrivalent live attenuated influenza vaccine, an antiviral agent, or inactivated influenza vaccine. In another embodiment, the at least one other therapeutically active agent is selected from oseltamivir, zanamivir, rimantadine, or amantadine. In one embodiment, the RIP1 kinase-mediated disease or disorder treated with these agents is influenza.

In one embodiment of this invention, the at least one other therapeutically active agent is selected from a β-Lactam, nafcillin, sulfamethoxazolylm, trimethoprim, sulfasalazine, acetyl sulfisoxazolyl, and vancomycin. In one embodiment, the RIP1 kinase-mediated disease or disorder treated with these agents is a staphylococcus infection.

In one embodiment of this invention, the at least one other therapeutically active agent is selected from a monoclonal antibody, a polyclonal anti-T-cell antibody, an anti-thymocyte gamma globulin-equine antibody, an antithymocyte globulin-rabbit antibody, an anti-CD40 antagonist, a JAK inhibitor, and an anti-TCR murine mAb. In another embodiment, the at least one other therapeutically active agent is selected from muromonab-CD3, ASKP-1240, ASP015K, and TOL101. In one embodiment, the RIP1 kinase-mediated disease or disorder treated with these agents is transplant rejection.

In one embodiment of this invention, the at least one other therapeutically active agent is selected from a topical immunomodulator or calcineurin inhibitor, a topical corticosteroid, an oral corticosteroid, an interferon gamma, an antihistamine, or an antibiotic. In another embodiment, the at least one other therapeutically active agent is selected from pimecrolimus, tacrolimus, hydrocortizone, betamethasone, flurandrenolide, fluticasone, triamcinolone, fluocinonide, clobetasol, hydrocortisone, methylprednisolone, prednisolone, an interferon alpha protein, a recombinant synthetic type I interferon, interferon alpha-2a, interferon alpha-2b, hydroxyzine, diphenhydramine, flucloxacillin, dicloxacillin, and erythromycin. In one embodiment, the RIP1 kinase-mediated disease or disorder treated with these agents is atopic dermatitis.

In another embodiment, a compound that inhibits RIP 1 kinase, particularly a compound of Formula (I) or Formula (II), or a pharmaceutically acceptable salt thereof, may be administered to a patient in need thereof, in combination with at least one other therapy and/or with at least one other active therapeutic agent that is considered standard of care (U.S. Department of Health and Human Services, Agency for Healthcare Research and Quality, National Guideline Clearinghouse, https://www.guideline.gov/ and World Health Organization, http://www.who.int/management/quality/standards/en/) for any of the diseases and/or disorders recited herein.

A compound that inhibits RIP 1 kinase, particularly a compound of Formula (I) or Formula (II), or a pharmaceutically acceptable salt thereof, may be administered to a patient in need thereof, in combination with at least one other active therapeutic agent, wherein the at least one other active therapeutic agent is: a corticosteroid [administered orally, topically, by injection, or as a suppository; prednisone, methylprednisolone, prednisolone, budesonide, betamethasone, dexamethasone, hydrocortisone, triamcinolone, fluticasone (fluticasone furoate, fluticasone propionate), fludroxycortide (flurandrenolide, flurandrenolone), fluocinonide, clobetasol (clobetasol propionate)], an anti-TNF biologic agent (etanecerpt, adalimumab, infliximab, certolizumab, golimumab), an other biologic agent (vedolizumab, etrolizumab, eldelumab, or bertilimumab), biosimilars to any of the above biologic agents, a PDE-4 inhibitor (apremilast), 5-aminosalicyclic acid (mesalazine/mesalamine; sulfasalazine, balsalazide), a DMARD (a disease-modifying anti-rheumatic drug: methotrexate, hydroxychloroquine, sulfasalazine, leflunomide), a thiopurine (azathioprine, mercaptopurine), a JAK inhibitor (tofacitinib, Baracitinib), an NSAID (aspirin, acetaminophen, ibuprofen, naproxen (naproxen sodium), etodolac, celecoxib, diclofenac, meloxicam), an anti-IL6 biologic agent (tocilizumab), an anti-IL1 biologic agent (anakinra, canakinumab, rilonacept), an anti-IL12 or IL23 biologic agent (ustekinumab, risankizumab, guselkumab, tildrakizumab), an anti-CD6 biologic agent (itolizumab), an anti-integrin agent (natalizumab (Tysabri®), etrolizumab), an anti-IL17 biologic agent (secukinumab, ixekizumab, brodalumab), an anti-CD22 biologic agent (epratuzumab), an anti-CD20 biologic agent (rituximab, ofatumumab), an anti-CD20 or CD4 biologic agent and other cytokine inhibitor or biologic to T-cell or B-cell receptors or interleukins, T cell inhibitors (abatacept) a calcineurin inhibitor (cyclosporine, pimecrolimus, tacrolimus), acitretin, fumaric acid, dimethyl fumarate, cyclophosphamide, cyclosporine (or ciclosporin), methotrexate, mycophenolic acid (or mycophenolate mofetil), topical vitamin D (calcipotriol or calcipotriene), an mTOR inhibitor (temsirolimus, everolimus), a Syk inhibitor (fostamatinib), an anti-IFNa biologic agent (sifalimumab), a retinoid (tazarotene), coal tar preparations, aryl hydrocarbon receptor agonist or modulating agent (tapinarof), hydroxyurea, 6-tioguanineor light therapy with or without psoralen.

Examples of other active therapeutic agents that may be used in combination with a compound of this invention for the treatment of ulcerative colitis and/or Crohn's disease include vedolizumab, etrolizumab, eldelumab, or bertilimumab.

Examples of other suitable biologic agents include abatacept, belimumab, and alicafosen. Examples of other active therapeutic agent that may be used in combination with a compound of this invention include baracitinib and Remestemcel-L.

A compound that inhibits RIP 1 kinase, particularly a compound of Formula (I) or Formula (II), or a pharmaceutically acceptable salt thereof, may be administered to a pediatric or an adult patient in need thereof, in combination with at least one other therapy, for example, in combination with UVA and/or UVB phototherapy as indicated for the treatment of psoriasis.

In the treatment of pediatric and/or adult psoriasis, a compound that inhibits RIP 1 kinase, particularly a compound of Formula (I) or Formula (II), or a pharmaceutically acceptable salt thereof, may be administered to reduce the signs and symptoms including body surface area, pruritis, nail disease, and scalp involvement, and to improve quality of life, in pediatric and/or adult patients with moderate to severe psoriasis.

In the treatment of pediatric and/or adult psoriasis, a compound that inhibits RIP 1 kinase, particularly a compound of Formula (I) or Formula (II), or a pharmaceutically acceptable salt thereof, may be administered as initial treatment or after treatment with another agent in pediatric and/or adult patients with moderate to severe psoriasis.

In the treatment of pediatric and/or adult psoriasis, a compound that inhibits RIP 1 kinase, particularly a compound of Formula (I) or Formula (II), or a pharmaceutically acceptable salt thereof, may be administered to maintain reductions in signs and symptoms and improvements in quality of life after treatment with another agent in pediatric and/or adult patients with moderate to severe psoriasis.

A compound that inhibits RIP 1 kinase, particularly a compound of Formula (I) or Formula (II), or a pharmaceutically acceptable salt thereof, may be administered for the treatment of moderately to severely active rheumatoid arthritis.

In the treatment of rheumatoid arthritis, a compound that inhibits RIP 1 kinase, particularly a compound of Formula (I) or Formula (II), or a pharmaceutically acceptable salt thereof, may be administered to reduce the signs and symptoms, to induce a major clinical response, to inhibit the progression of structural damage, or to improve physical function in a patient, particularly an adult patient with moderately to severely active rheumatoid arthritis.

In the treatment of rheumatoid arthritis, a compound that inhibits RIP 1 kinase, particularly a compound of Formula (I) or Formula (II), or a pharmaceutically acceptable salt thereof, may be administered alone or in combination with methotrexate or other non-biologic disease-modifying anti-rheumatic drugs (DMARDs). In a specific embodiment, a compound that inhibits RIP 1 kinase, particularly a compound of Formula (I) or Formula (II), or a pharmaceutically acceptable salt thereof, may be administered alone or in combination with methotrexate, or corticosteroids in the treatment of rheumatoid arthritis.

In the treatment of juvenile idiopathic arthritis (IA), a compound that inhibits RIP 1 kinase, particularly a compound of Formula (I) or Formula (II), or a pharmaceutically acceptable salt thereof, may be administered to reduce the signs and symptoms of moderately to severely active polyarticular juvenile idiopathic arthritis in patients 2 years of age and older.

In the treatment of juvenile idiopathic arthritis, particularly polyarticular juvenile idiopathic arthritis, a compound that inhibits RIP 1 kinase, particularly a compound of Formula (I) or Formula (II), or a pharmaceutically acceptable salt thereof, may be administered alone or in combination with methotrexate.

In the treatment of psoriatic arthritis, a compound that inhibits RIP 1 kinase, particularly a compound of Formula (I) or Formula (II), or a pharmaceutically acceptable salt thereof, may be administered to reduce the signs and symptoms, inhibiting the progression of structural damage, of active arthritis, and/or to improve physical function in adult patients with psoriatic arthritis.

In the treatment of psoriatic arthritis, a compound that inhibits RIP 1 kinase, particularly a compound of Formula (I) or Formula (II), or a pharmaceutically acceptable salt thereof, may be administered alone or in combination with methotrexate, corticosteroids, or other non-biologic disease-modifying anti-rheumatic drugs (DMARDs).

In a specific embodiment, a compound that inhibits RIP 1 kinase, particularly a compound of Formula (I) or Formula (II), or a pharmaceutically acceptable salt thereof, may be administered alone or in combination with methotrexate for the treatment of psoriatic arthritis.

In the treatment of psoriatic arthritis, a compound that inhibits RIP 1 kinase, particularly a compound of Formula (I) or Formula (II), or a pharmaceutically acceptable salt thereof, may be administered to a patient, particularly an adult patient with moderate to severe chronic plaque psoriasis, who is a candidate for systemic therapy or phototherapy.

In the treatment of axial Spondyloarthritis and ankylosing spondylitis, a compound that inhibits RIP 1 kinase, particularly a compound of Formula (I) or Formula (II), or a pharmaceutically acceptable salt thereof, may be administered to reduce the signs and symptoms of active ankylosing spondylitis in a patient, either an adult or a pediatric patient, in need thereof.

In the treatment of axial Spondyloarthritis and ankylosing spondylitis, a compound that inhibits RIP 1 kinase, particularly a compound of Formula (I) or Formula (II), or a pharmaceutically acceptable salt thereof, may be administered alone or in combination with methotrexate, corticosteroids, or other non-biologic disease-modifying anti-rheumatic drugs (DMARDs).

In the treatment of Crohn's Disease, a compound that inhibits RIP 1 kinase, particularly a compound of Formula (I) or Formula (II), or a pharmaceutically acceptable salt thereof, may be administered to reduce the signs and symptoms of Crohn's disease. A compound that inhibits RIP 1 kinase, particularly a compound of Formula (I) or Formula (II), or a pharmaceutically acceptable salt thereof, may be administered to induce or maintain a clinical response (clinical remission) in a patient, particularly an adult patient with moderately to severely active Crohn's disease.

In the treatment of Crohn's Disease, a compound that inhibits RIP 1 kinase, particularly a compound of Formula (I) or Formula (II), or a pharmaceutically acceptable salt thereof, may be administered to reduce the signs and symptoms of Crohn's disease. A compound that inhibits RIP 1 kinase, particularly a compound of Formula (I) or Formula (II), or a pharmaceutically acceptable salt thereof, may be administered to induce or maintain a clinical response (clinical remission) in a patient, particularly a pediatric patient 6 years of age and older with moderately to severely active Crohn's disease.

A compound that inhibits RIP 1 kinase, particularly a compound of Formula (I) or Formula (II), or a pharmaceutically acceptable salt thereof, may be administered to reduce the signs and symptoms of Crohn's disease, particularly, moderately to severely active Crohn's disease, in a patient who has had an inadequate response to corticosteroids or immunomodulators such as azathioprine, 6-mercaptopurine, or methotrexate.

A compound that inhibits RIP 1 kinase, particularly a compound of Formula (I) or Formula (II), or a pharmaceutically acceptable salt thereof, may be administered to treat a patient, particularly an adult patient or a pediatric patient 6 years and older, with moderately to severely active ulcerative colitis.

In the treatment of ulcerative colitis, a compound that inhibits RIP 1 kinase, particularly a compound of Formula (I) or Formula (II), or a pharmaceutically acceptable salt thereof, may be administered to induce and/or sustain clinical remission in a patient, particularly an adult patient or a pediatric patient 6 years and older, with moderately to severely active ulcerative colitis.

A compound that inhibits RIP 1 kinase, particularly a compound of Formula (I) or Formula (II), or a pharmaceutically acceptable salt thereof, may be administered to induce and/or sustain a clinical response (clinical remission) in a patient, particularly a patient with moderately to severely active ulcerative colitis, who has had an inadequate response to immunosuppressants such as aminosalicylates, corticosteroids, azathioprine or 6-mercaptopurine (6-MP).

A compound that inhibits RIP 1 kinase, particularly a compound of Formula (I) or Formula (II), or a pharmaceutically acceptable salt thereof, may be administered for the treatment of moderate to severe hidradenitis suppurativa.

A compound that inhibits RIP 1 kinase, particularly a compound of Formula (I) or Formula (II), or a pharmaceutically acceptable salt thereof, may be administered for the treatment of uveitis, particularly non-infectious intermediate, posterior and panuveitis, in a patient, particularly an adult patient, in need thereof.

Accordingly, one embodiment of this invention is directed to a method of inhibiting RIP1 kinase comprising contacting a cell with a compound of the invention. Another embodiment of this invention is a method of inhibiting RIP1 kinase comprising contacting a cell with a compound of Formula (I) or Formula (II) or a pharmaceutically acceptable salt thereof. A particular embodiment of this invention is to a method of inhibiting RIP1 kinase comprising contacting a cell with a compound of Formula (II) or Formula (II) or a pharmaceutically acceptable salt thereof.

In another embodiment, the invention is directed to a method of treating a RIP1 kinase-mediated disease or disorder (for example, a disease or disorder recited herein) comprising administering a therapeutically effective amount of a compound of Formula (I) or Formula (II), or a pharmaceutically acceptable salt thereof, to a human in need thereof. In a particular embodiment, the invention is directed to a method of treating a RIP1 kinase-mediated disease or disorder (for example, a disease or disorder recited herein) comprising administering a therapeutically effective amount of a compound disclosed herein, or a pharmaceutically acceptable salt thereof, to a human in need thereof.

This invention also provides a compound of Formula (I) or Formula (II), or a pharmaceutically acceptable salt thereof, for use in therapy. This invention provides a compound of Formula (I) or Formula (II), or a pharmaceutically acceptable salt thereof, for use in the treatment of a RIP1 kinase-mediated disease or disorder (for example, a disease or disorder recited herein). Specifically, this invention provides a compound described herein, or a pharmaceutically acceptable salt thereof, for use in therapy.

In another embodiment, this invention provides a compound of the invention for use in the treatment of a RIP1 kinase-mediated disease or disorder, specifically, a disease or disorder recited herein. This invention provides a compound described herein, or a pharmaceutically acceptable salt thereof, for use in the treatment of a RIP1 kinase-mediated disease or disorder, specifically, a disease or disorder recited herein.

This invention specifically provides for the use of a compound of Formula (I) or Formula (II), or a pharmaceutically acceptable salt thereof, as an active therapeutic substance. More specifically, this invention provides for the use of the compounds described herein for the treatment of a RIP1 kinase-mediated disease or disorder, specifically, a disease or disorder recited herein. Accordingly, the invention provides for the use of a compound of Formula (I) or Formula (II) as an active therapeutic substance in the treatment of a human in need thereof with a RIP1 kinase-mediated disease or disorder, specifically, a disease or disorder recited herein.

The invention further provides for the use of a compound of Formula (I) or Formula (II), or a pharmaceutically acceptable salt thereof, in the manufacture of a medicament for use in the treatment of a RIP1 kinase-mediated disease or disorder, for example the diseases and disorders recited herein. Specifically, the invention also provides for the use of a compound described herein, or a pharmaceutically acceptable salt thereof, in the manufacture of a medicament for use in the treatment of a RIP1 kinase-mediated disease or disorder, for example the diseases and disorders recited herein.

A therapeutically “effective amount” is intended to mean that amount of a compound that, when administered to a patient in need of such treatment, is sufficient to effect treatment, as defined herein. Thus, e.g., a therapeutically effective amount of a compound of Formula (I) or Formula (II), or a pharmaceutically acceptable salt thereof, is a quantity of an inventive agent that, when administered to a human in need thereof, is sufficient to modulate and/or inhibit the activity of RIP1 kinase such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmacokinetic properties), disease or disorder and its severity and the specific composition and method being used, but can nevertheless be determined by one of skill in the art.

“Treating” or “treatment” is intended to mean at least the mitigation of a disease or disorder in a patient. The methods of treatment for mitigation of a disease or disorder include the use of the compounds in this invention in any conventionally acceptable manner, for example for prevention, retardation, prophylaxis, therapy or cure of a RIP1 kinase mediated disease or disorder, as described hereinabove.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the disease or disorder being treated, the severity of the disease or disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Total daily dosages range from 1 mg to 2000 mg.

For use in therapy, the compounds of the invention will be normally, but not necessarily, formulated into a pharmaceutical composition prior to administration to a patient. Accordingly, the invention also is directed to pharmaceutical compositions comprising a compound of the invention and one or more pharmaceutically acceptable excipients. The invention is directed to a pharmaceutical composition comprising a compound of Formula (I) or Formula (II), or a pharmaceutically acceptable salt thereof, and one or more pharmaceutically acceptable excipients.

The invention is further directed to a pharmaceutical composition comprising a compound of Formula (I) or Formula (II), or a pharmaceutically acceptable salt thereof, and one or more pharmaceutically acceptable excipients and at least one other therapeutically active agent, specifically one or two other therapeutically active agents, more specifically one other therapeutically active agent.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. A dose of the pharmaceutical composition contains at least a therapeutically effective amount of a compound of this invention (i.e., a compound of Formula (I) or Formula (II), or a pharmaceutically acceptable salt, thereof). When prepared in unit dosage form, the pharmaceutical compositions may contain from 1 mg to 1000 mg of a compound of this invention.

As provided herein, unit dosage forms (pharmaceutical compositions) containing from 1 mg to 1000 mg of a compound of the invention may be administered one, two, three, or four times per day, preferably one, two, or three times per day, and more preferably, one or two times per day, to effect treatment of a RIP1 kinase-mediated disease or disorder.

As used herein, “pharmaceutically acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. Conventional dosage forms suitable for use with the compounds of this invention include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company). Accordingly, another embodiment of this invention is a method of preparing a pharmaceutical composition comprising the step of admixing a compound of Formula (I) or Formula (II), or a pharmaceutically acceptable salt, thereof, with one or more pharmaceutically acceptable excipients.

In one aspect, the invention is directed to a topical dosage form such as a cream, ointment, lotion, paste, or gel comprising an effective amount of a compound of the invention and one or more pharmaceutically acceptable excipients. Lipophilic formulations, such as anhydrous creams and ointments, generally will have a base derived from fatty alcohols, and polyethylene glycols. Additional additives include alcohols, non-ionic surfactants, and antioxidants. For ointments, the base normally will be an oil or mixture of oil and wax, e.g., petrolatum. Also, an antioxidant normally will be included in minor amounts. Because the compositions are applied topically and the effective dosage can be controlled by the total composition applied, the percentage of active ingredient in the composition can vary widely. Convenient concentrations range from 0.5% to 20%.

Topically applied gels can also be a foamable suspension gel comprising a compound of the invention, as an active agent, one or more thickening agents, and optionally, a dispersing/wetting agent, a pH-adjusting agent, a surfactant, a propellent, an antioxidant, an additional foaming agent, a chelating/sequestering agent, a solvent, a fragrance, a coloring agent, a preservative, wherein the gel is aqueous and forms a homogenous foam.

In one aspect, the invention is directed to a topical dosage form that can be administered by inhalation, that is, by intranasal and oral inhalation administration. Appropriate dosage forms for such administration, such as an aerosol formulation or a metered dose inhaler, may be prepared by conventional techniques. Intranasal sprays may be formulated with aqueous or non-aqueous vehicles with the addition of agents such as thickening agents, buffer salts or acid or alkali to adjust the pH, isotonicity adjusting agents or anti-oxidants. Solutions for inhalation by nebulization may be formulated with an aqueous vehicle with the addition of agents such as acid or alkali, buffer salts, isotonicity adjusting agents or antimicrobials.

Formulations for administration by inhalation or foamable gel often require the use of a suitable propellant. Capsules and cartridges of e.g. gelatin for use in an inhaler or insufflator may be formulated using a suitable powder base such as lactose or starch.

In another aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Definitions

As used herein, the term “alkyl” represents a saturated, straight or branched hydrocarbon group having the specified number of carbon atoms. The term “(C₁-C₄)alkyl” refers to an alkyl moiety containing from 1 to 4 carbon atoms. Exemplary alkyls include, but are not limited to methyl, ethyl, n-propyl, isopropyl, n-butyl, isobutyl, s-butyl, and t-butyl.

As used herein, “5-6-membered heteroaryl” represents an aromatic monocyclic group containing 5 or 6 ring atoms, including at least one carbon atom and 1 to 4 heteroatoms independently selected from nitrogen, oxygen and sulfur. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Examples of 5-membered heteroaryl groups include furyl (furanyl), thienyl, pyrrolyl, imidazolyl, pyrazolyl, triazolyl, tetrazolyl, thiazolyl, isothiazolyl, thiadiazolyl, oxazolyl, isoxazolyl, oxadiazolyl and oxo-oxadiazolyl. Selected 6-membered heteroaryl groups include pyridinyl, oxo-pyridyl, pyrazinyl, pyrimidinyl, pyridazinyl and triazinyl.

The term “halogen” represents chloro, fluoro, bromo, or iodo substituents.

As used herein, the term “cyano” refers to the group —CN.

The term “independently” means that where more than one substituent is selected from a number of possible substituents, those substituents may be the same or different.

As used herein, the term “optionally” means that the subsequently described event(s) may or may not occur, and includes both event(s) that occur and event(s) that do not occur.

The term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The compounds of this invention contain one or more asymmetric centers (also referred to as a chiral center), such as a chiral carbon, or a chiral —SO— moiety. The stereochemistry of the chiral carbon center present in compounds of this invention is generally represented in the compound names and/or in the chemical structures illustrated herein. Compounds of this invention containing one or more chiral centers may be present as racemic mixtures, diastereomeric mixtures, enantiomerically enriched mixtures, diastereomerically enriched mixtures, or as enantiomerically or diastereomerically pure individual stereoisomers.

In those instances where the stereochemistry of the chiral carbon center present in compounds of this invention is not represented in the compound name or in the accompanying chemical structure, it will be understood that the compound is present as a mixture of enantiomers or diastereomers. It is understood that one skilled in the art can obtain either the (R) or (S) isomer of any stereoisomeric compound mixture described herein using the resolution techniques described herein or using other conventional resolution techniques.

Individual stereoisomers of a compound described herein may be resolved (or mixtures of stereoisomers may be enriched) using methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The alternative definitions for the various groups and substituent groups of Formula(s) (I) and/or (II) provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

As used herein, the terms “compound(s) of the invention” or “compound(s) of this invention” mean a compound of Formula(s) (I) and/or (II) as defined herein, in any form, i.e., any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms.

Accordingly, included within the present invention are the compounds of Formulas (I) and (II), as defined herein, in any salt or non-salt form and any physical form thereof, and mixtures of various forms. While such are included within the present invention, it will be understood that the compounds of Formulas (I) and (II), as defined herein, in any salt or non-salt form, and in any physical form thereof, may have varying levels of activity, different bioavailabilities and different handling properties for formulation purposes.

“Treating” or “treatment” is intended to mean at least the mitigation of a disease or disorder in a patient. The methods of treatment for mitigation of a disease or disorder include the use of the compounds in this invention in any conventionally acceptable manner, for example for prevention, retardation, prophylaxis, therapy or cure of a RIP1 kinase mediated disease or disorder, as described hereinabove.

As used herein, the term “cancer,” refers to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.”

A therapeutically “effective amount” is intended to mean that amount of a compound that, when administered to a patient in need of such treatment, is sufficient to effect treatment, as defined herein. Thus, e.g., a therapeutically effective amount of a compound of Formula (I) or Formula (II), or a pharmaceutically acceptable salt thereof, is a quantity of an inventive agent that, when administered to a human in need thereof, is sufficient to modulate and/or inhibit the activity of RIP1 kinase such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmacokinetic properties), disease or disorder and its severity and the specific composition and method being used, but can nevertheless be determined by one of skill in the art.

As used herein, “pharmaceutically acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

### Compound Preparation

### Generic Synthesis Schemes

The compounds of this invention may be made by a variety of methods, including well-known standard synthetic methods. Illustrative general synthetic methods are set out below and then specific compounds of the invention are prepared in the working examples. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, Protecting Groups in Organic Synthesis, John Wiley & Sons (1991) incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Starting materials are commercially available or are made from commercially available starting materials using methods known to those skilled in the art.

As will be understood by the skilled chemist, references to preparations carried out in a similar manner to, or by the general method of, other preparations, may encompass variations in routine parameters such as time, temperature, workup conditions, minor changes in reagent amounts, etc. The syntheses of intermediates provided in the Examples herein are applicable for producing intermediates of the invention having a variety of R groups employing appropriate precursors, which are protected if needed, to achieve compatibility with the reactions described.

Compounds of Formula I can be prepared according to Scheme 1. An aldehyde of Formula A can be reacted with 2,2-dimethyl-1,3-dioxane-4,6-dione and tert-butyl N-hydroxycarbamate in the presence of 1,4-diazabicyclo[2.2.2]octane to afford an isoxazolidinone of Formula B. Treatment of a compound of Formula B with lithium borohydride gives an alcohol of Formula C. A compound of Formula C can be cyclized under Mitsunobu conditions, and the Boc group of the resulting isoxazolidine (D) can be subsequently removed upon treatment with acid to afford a compound of Formula E. An isoxazolidine of Formula E can be coupled with an appropriately substituted carboxylic acid or acid chloride to afford a compound of Formula I.

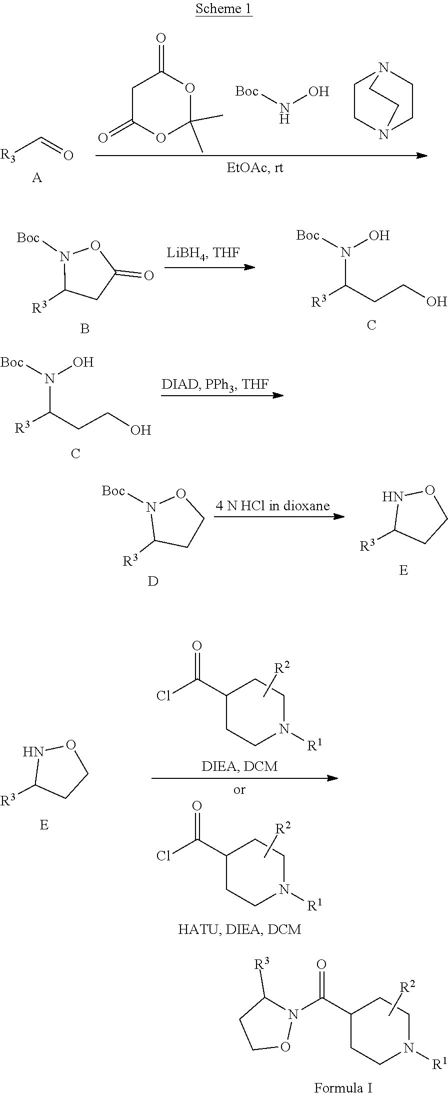

Alternatively, compounds of Formula I can be prepared according to Scheme 2. An isoxazolidine of Formula E, prepared according to Scheme 1, can be coupled with a Boc protected piperidine-4-carboxylic acid to give a compound of Formula F. The Boc group of a compound of Formula F can be removed upon treatment with acid, and the resulting amine salt (G) can be reacted with a heteroaryl halide in the presence of base to afford a compound of Formula I.

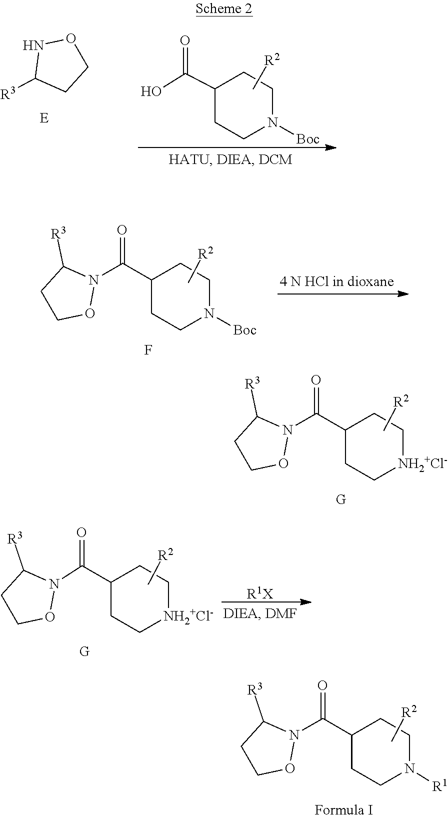

Compounds of Formula II can be prepared according to Scheme 3, wherein a compound of Formula I can be subjected to chiral separation methods to afford a compound of Formula II.

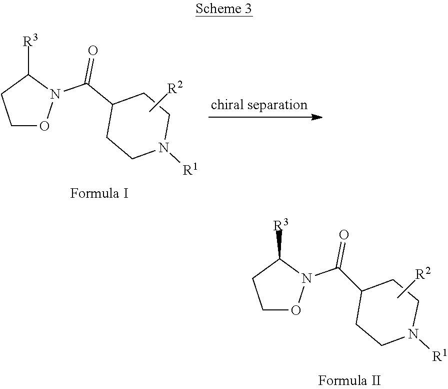

Alternatively, compounds of Formula II can be prepared according to Scheme 4. An aldehyde of Formula A can be condensed with acetaldehyde to afford an α,β-unsaturated aldehyde of Formula H. A compound of Formula H can be reacted with Boc-protected hydroxylamine and subsequently reduced to yield a compound of Formula J. A compound of Formula J can be cyclized under Mitsunobu conditions, and the Boc group of the resulting isoxazolidine can be subsequently removed upon treatment with acid to afford a compound of Formula M. A compound of Formula M can be further transformed according to the methods outlined in Scheme 1 or Scheme 2 to afford a compound of Formula II.

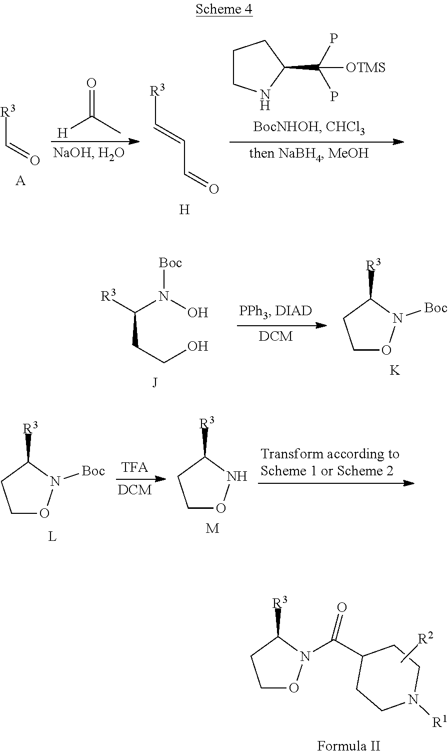

### EXPERIMENTALS

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

The reactions described herein are applicable for producing compounds of Formulas (I) and (II) having a variety of different substituent groups (e.g., R¹, R², etc.), as defined herein. The skilled artisan will appreciate that if a particular substituent is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, N Y, 1999.

Names for the intermediate and final compounds described herein were generated using the naming program in ChemDraw, Struct=Name Pro 12.0, as part of ChemBioDraw Ultra, available from CambridgeSoft. 100 CambridgePark Drive, Cambridge, Mass. 02140 USA (www.cambridgesoft.com).

It will be appreciated by those skilled in the art that in certain instances these programs may name a structurally depicted compound as a tautomer of that compound. It is to be understood that any reference to a named compound or a structurally depicted compound is intended to encompass all tautomers of such compounds and any mixtures of tautomers thereof.

¹H NMR spectra were recorded in either CDCl₃, CD₃OD-d₄, or DMSO-d₆ on either a Bruker Advance III 400 MHz, Bruker Fourier 300 MHz, Bruker Mercury Plus 300 NMR at 400 MHz, or 400 NMR Spectrometer equipped with a Bruker 400 BBO probe. The internal standard used was either tetramethylsilane or the residual protonated solvent at 7.26 ppm for CDCl₃, 3.31 ppm for CD₃OD-d₄, or 2.50 ppm for DMSO-d₆. Chemical shifts are reported in parts per million (ppm). Abbreviations for NMR data are as follows: s=singlet, d=doublet, t=triplet, m=multiplet, br s=broad singlet, dd=doublet of doublets, dt=doublet of triplets, tt=triplet of triplets, ddd=doublet of doublet of doublets, sextuplet of d=sextuplet of doublets. J indicates the ¹H NMR coupling constant measured in Hertz.

Mass spectrum was recorded on a Waters ZQ mass spectrometer using alternative-scan positive and negative mode electrospray ionisation. Cone voltage: 30V.

TABLE 1
LC/MS Analytical Method

| | |
|---|---|
| LC/MS | The column used was a Kinetex EVO, 2.6 μm, 3.0 × 50 mm. A |
| Method 1 | linear gradient was applied, starting at 90% A (A: 0.03% NH₃•H₂O |
| | in water) and ending at 95% B (B: MeCN) over 2.6 min with a total |
| | run time of 3.0 min. The column temperature was at 40° C. with the |
| | flow rate of 1.2 mL/min. |
| LC/MS | The column used was a Shim-pack XR-ODS. 2.2 μm, 3.0 × 50 mm. |
| Method 2 | A linear gradient was applied, starting at 95% A (A: 0.05% TFA in |
| | water) and ending at 100% B (B: 0.05% TFA in MeCN) over 1.7 |
| | min with a total run time of 2.0 min. The column temperature was |
| | at 40° C. with the flow rate of 1.2 mL/min. |
| LC/MS | The column used was a Shim-pack XR-ODS, 2.2 μm, 3.0 × 50 mm. |
| Method 3 | A linear gradient was applied, starting at 95% A (A: 0.05% TFA in |
| | water) and ending at 100% B (B: 0.05% TFA in MeCN) over 2.7 |
| | min with a total run time of 3.0 min. The column temperature was |
| | at 40° C. with the flow rate of 1.2 mL/min. |
| LC/MS | The column used was an Ascentis Express C18, 2.7 μm, 3.0 × 50 |
| Method 4 | mm. A linear gradient was applied, starting at 95% A (A: 0.05% |
| | TFA in water) and ending at 95% B (B: 0.05% TFA in MeCN) over |
| | 1.7 min with a total run time of 2.0 min. The column temperature |
| | was at 40° C. with the flow rate of 1.5 mL/min. |
| LC/MS | The column used was an Ascentis Express C18, 2.7 μm, 3.0 × 50 |
| Method 5 | mm. A linear gradient was applied, starting at 95% A (A: 0.05% |
| | TFA in water) and ending at 95% B (B: 0.05% TFA in MeCN) over |
| | 2.10 min with a total run time of 2.6 min. The column temperature |
| | was at 40° C. with the flow rate of 1.5 mL/min. |
| LC/MS | The column used was a CORTECS C18, 2.7 μm, 2.1 × 50 mm. A |
| Method 6 | linear gradient was applied, starting at 95% A (A: 0.09% FA in |
| | water) and ending at 100% B (B: 0.1% FA in MeCN) over 1.6 min |
| | with a total run time of 1.8 min. The column temperature was at |
| | 40° C. with the flow rate of 1.2 mL/min. |
| LC/MS | The column used was a CORTECS C18, 2.7 μm, 2.1 × 50 mm. A |
| Method 7 | linear gradient was applied, starting at 95% A (A: 0.09% FA in |
| | water) and ending at 95% B (B: 0.1% FA in MeCN) over 2.6 min |
| | with a total run time of 3.0 min. The column temperature was at |
| | 40° C. with the flow rate of 1.2 mL/min. |
| LC/MS | The column used was an Agilent Poroshell HPH-C18, 2.7 μm, |
| Method 8 | 3.0 × 50 mm. A linear gradient was applied, starting at 90% A (A: 6.5 |
| | mM NH₄HCO₃ + NH₃•H₂O in water) and ending at 95% B (B: |
| | MeCN) over 2.7 min with a total run time of 3.0 min. The column |
| | temperature was at 40° C. with the flow rate of 1.2 mL/min. |
| LC/MS | The column used was a CORTECS C18, 2.7 μm, 2.1 × 50 mm. A |
| Method 9 | linear gradient was applied, starting at 95% A (A: 0.05% TFA in |
| | water) and ending at 100% B (B: 0.05% TFA in MeCN) over 2.8 |
| | min with a total run time of 3.0 min. The column temperature was |
| | at 40° C. with the flow rate of 1.2 mL/min. |
| LC/MS | The column used was a Shim-pack XR-ODS, 2.2 μm, 3.0 × 50 mm. |
| Method 10 | A linear gradient was applied, starting at 95% A (A: 0.05% TFA in |
| | water) and ending at 95% B (B: 0.05% TFA in MeCN) over 2.7 min |
| | with a total run time of 3 min. The column temperature was at 40° |
| | C. with the flow rate of 1.2 mL/min. |
| LC/MS | The column used was an Ascentis Express C18, 2.7 μm, 3.0 × 50 |
| Method 11 | mm. A linear gradient was applied, starting at 95% A (A: 0.05% |
| | TFA in water) and ending at 100% B (B: 0.05% TFA in MeCN) |
| | over 2.8 min with a total run time of 3.0 min. The column |
| | temperature was at 40° C. with the flow rate of 1.5 mL/min. |
| LC/MS | The column used was a Kinelex 2.6 μm XB-C18, 2.6 μm, 3.0 × 50 |
| Method 12 | mm. A linear gradient was applied, starting at 95% A (A: 0.05% |
| | TFA in water) and ending at 100% B (B: 0.05% TFA in MeCN) |
| | over 2.8 min with a total run time of 3.0 min. The column |
| | temperature was at 45° C. with the flow rate of 1.5 mL/min. |
| LC/MS | The column used was an Agilent Poroshell HPH-C18, 2.7 μm, |
| Method 13 | 3.0 × 50 mm. A linear gradient was applied, starting at 90% A (A: 6.5 |
| | mM NH₄HCO₃ + NH₃•H₂O in water) and ending at 95% B (B: |
| | MeCN) over 1.7 min with a total run time of 2.0 min. The column |
| | temperature was at 40° C. with the flow rate of 1.2 mL/min. |
| LC/MS | The column used was a Kinelex XB-C18, 2.6 μm, 3.0 × 50 mm. A |
| Method 14 | linear gradient was applied, starting at 95% A (A: 0.05% TFA in |
| | water) and ending at 100% B (B: 0.05% TFA in MeCN) over 1.8 |
| | min with a total run time of 2.0 min. The column temperature was |
| | at 40° C. with the flow rate of 1.5 mL/min. |
| LC/MS | The column used was an Ascentis Express C18, 2.7 μm, 3.0 × 50 |
| Method 15 | mm. A linear gradient was applied, starting at 95% A (A: 0.05% |
| | TFA in water) and ending at 95% B (B: 0.05% TFA in MeCN) over |
| | 1.8 min with a total run time of 2.0 min. The column temperature |
| | was at 40° C. with the flow rate of 1.5 mL/min. |
| LC/MS | The column used was an Ascentis Express C18, 2.7 μm, 3.0 × 50 |
| Method 16 | mm. A linear gradient was applied, starting at 95% A (A: 0.05% |
| | TFA in water) and ending at 95% B (B: 0.05% TFA in MeCN) over |
| | 2.8 min with a total run time of 3.0 min. The column temperature |
| | was at 40° C. with the flow rate of 1.5 mL/min. |
| LC/MS | The column used was a Kinetex 2.6 μm EVO C18 100A, 2.6 μm, |
| Method 17 | 3.0 × 50 mm. A linear gradient was applied, starting at 90% A (A: |
| | 5 mmol NH₄HCO₃ in water) and ending at 95% B (B: MeCN) over |
| | 4.9 min with a total run time of 5.3 min. The column temperature |
| | was at 40° C. with the flow rate of 1.2 mL/min. |
| LC/MS | The column used was a Kinetex 2.6 μm EVO C18 100A, 2.6 μm, |
| Method 18 | 3.0 × 50 mm. A linear gradient was applied, starting at 90% A (A: |
| | 5 mmol NH₄HCO₃ in water) and ending at 95% B (B: MeCN) over |
| | 2.75 min with a total run time of 3.0 min. The column temperature |
| | was at 40° C. with the flow rate of 1.2 mL/min. |
| LC/MS | The column used was a Kinelex 2.6 μm XB-C18, 2.6 μm, 3.0 × 50 |
| Method 19 | mm. A linear gradient was applied, starting at 95% A (A: 0.05% |
| | TFA in water) and ending at 100% B (B: 0.05% TFA in MeCN) |
| | over 4.5 min with a total run time of 5.0 min. The column |
| | temperature was at 45° C. with the flow rate of 1.5 mL/min. |
| LC/MS | The column used was an Ascientis Express C18, 2.7 μm, 2.1 mm × |
| Method 20 | 50 mm. A linear gradient was applied, starting at 90% A (A: 0.1% |
| | FA in water) and ending at 100% B (B: 0.1% FA in ACN) over 1.1 |
| | min with a total run time of 2.0 min. The column temperature was |
| | at 40° C. with the flow rate of 1.0 mL/min. |
| LC/MS | The column used was a Poroshell HPH-C18, 2.7 μm, 3.0 mm × 50 |
| Method 21 | mm. A linear gradient was applied, starting at 90% A (A: 6.5 mM |
| | NH₄HCO₃ + NH₃H₂O in water) and ending at 95% B (B: MeCN) |
| | over 4.8 min with a total run time of 5.1 min. The column |
| | temperature was at 45° C. with the flow rate of 1.2 mL/min. |
| LC/MS | The column used was a CORTECS C18, 2.7 μm, 2.1 × 50 mm. A |
| Method 22 | linear gradient was applied, starting at 95% A (A: 0.05% TFA in |
| | water) and ending at 100% B (B: 0.05% TFA in MeCN) over 4.50 |
| | min with a total run time of 5 min. The column temperature was at |
| | 45° C. with the flow rate of 1.5 mL/min. |
| LCMS | (x-95%-4 min) HPLC (AGILENT1260/6120B) was conducted on a |
| Method 23 | Dikma C18 column (5 μm 30 × 4.6 mm id) eluting with (0.02% |
| | NH₄OAc + 5% acetonitrile in water) (solvent A) and acetonitrile |
| | (solvent B), using the following elution gradient 0-4 min.: 0- |
| | 0.2 min.: x % B, 0.2-2.0 min.: x %-95% B, 2.0-3.5 min.: 95% B, 3.5- |
| | 3.6 min.: 95%-x % B, 3.6-4.0 min.: x % B at a flow rate of 1.5 mL/min. |
| | at 40° C. |
| LCMS | (x-95%-6.5 min) HPLC (AGILENT1260/6120B or SHIMADZU |
| Method 24 | LCMS2020) was conducted on a Waters XBridge C18 column |
| | (5 μm 50 × 4.6 mm id) eluting with (0.02% NH₄OAc in water) |
| | (solvent A) and acetonitrile (solvent B), using the following elution |
| | gradient: 0-0.5 min.: x % B, 0.5-4.5 min.: x %-95% B, 4.5-6.0 min.: |
| | 95% B, 6.0-6.1 min.: 95%-x % B, 6.1-6.5 min.: x % B at a flow rate |
| | of 1.5 mL/min. at 40° C. |
| LCMS | (x-y %-6.5 min) HPLC (AGILENT1260/6120B or SHIMADZU |
| Method 25 | LCMS2020) was conducted on a Phenomenex C18 column (5 μm |
| | 50 × 4.6 mm id) eluting with (0.02% NH₄OAc in water) (solvent |
| | A) and acetonitrile (solvent B), using the following elution gradient: |
| | 0-0.5 min.: x % B, 0.5-4.5 min.: x %-y % B, 4.5-5.0 min.: y %-95% |
| | B, 5.0-6.0 min.: 95% B, 6.0-6.1 min.: 95%-x % B, 6.1-6.5 min.: x % |
| | B at a flow rate of 1.5 mL/min. at 40° C. |

TABLE 2
UPLC-MS Analytical Method

| | |
|---|---|
| UPLC-MS | (x-95%-2.5 min) UPLC (Waters H-class/SQD) was conducted on a |
| Method 1 | BEH C18 column (1.7 μm 50 × 2.1 mm id) eluting with |
| | (0.02% NH4OAc + 5% acetonitrile in water) (solvent A) and |
| | acetonitrile (solvent B), using the following elution gradient 0-0.1 |
| | min.: x % B, 0.1-1.0 min.: x %-95% B, 1.0-2.1 min.: 95% B, 2.1-2.2 |
| | min.: 95%-x % B, 2.2-2.5 min.: x % B at a flow rate of 0.5 mL/min. |
| | at 40° C. |

TABLE 3
Chiral HPLC Analytical Method

| | |
|---|---|
| Chiral HPLC | on CHIRALPAK ® IG-3 was using 0.46 × 5 cm, 3 μm column, |
| Analytical | (hexane:DCM = 3:1) with 0.2% IPA/EtOH 80/20 at 254 nm, at a |
| Method 1 | flow rate of 1.0 mL/min. at 25° C. |
| Chiral HPLC | on CHIRALPAK ® IC-3 was using 0.46 × 5 cm, 3 μm column, |
| Analytical | hexane with 0.2% IPA/EtOH 50/50 at 254 nm, at a flow rate of 1.0 |
| Method 2 | mL/min. at 25° C. |
| Chiral HPLC | on CHIRALPAK ® IA-3 was using 0.46 × 5 cm, 3 μm column, |
| Analytical | 100% MeOH with 0.1% DEA at 254 nm, at a flow rate of 1.0 |
| Method 3 | mL/min. at 25° C. |
| Chiral HPLC | on Chiral Cellulose-SB was using 0.46 × 10 cm; 3 μm column, |
| Analytical | (Hex:DCM = 3:1)(0.1% DEA):EtOH = 70:30 at 254 nm, at a flow rate |
| Method 4 | of 1.0 mL/min. at 25° C. |
| Chiral HPLC | on Chiral Cellulose-SB was using 0.46 × 10 cm; 3 μm column, |
| Analytical | (Hex:DCM = 5:1)(0.1% DEA):EtOH = 70:30 at 254 nm, at a flow rate |
| Method 5 | of 1.0 mL/min. at 25° C. |
| Chiral HPLC | on Chiral Cellulose-SB was using 0.46 × 10 cm; 3 μm column, |
| Analytical | (Hex:DCM = 5:1)(0.1% DEA):IPA = 70:30 at 254 nm, at a flow rate |
| Method 6 | of 1.0 mL/min. at 25° C. |
| Chiral HPLC | on CHIRALPAK ® IC-3 was using 0.46 × 5 cm, 3 μm column, |
| Analytical | (Hex:DCM = 5:1)(0.1% DEA):EtOH = 50:50 at 254 nm, at a flow rate |
| Method 7 | of 1.0 mL/min. at 25° C. |
| Chiral HPLC | on CHIRALPAK ® IA-3 was using 0.46 × 5 cm, 3 μm column, |
| Analytical | (Hexane:DCM = 5:1) with (0.1% DEA)/EtOH = 50/50 at 254 nm, at |
| Method 8 | a flow rate of 1.0 mL/min. at 25° C. |
| Chiral HPLC | on Chiral Cellulose-SB was using 0.46 × 10 cm; 3 μm column, |
| Analytical | (Hex:DCM = 3:1)(0.1% DEA):IPA = 70:30 at 254 nm, at a flow rate |
| Method 9 | of 1.0 mL/min. at 25° C. |
| Chiral HPLC | on CHIRALPAK ® IF-3 was using 0.46 × 5 cm; 3 μm column, |
| Analytical | (Hexane:DCM = 5:1)(0.1% DEA):EtOH = 70:30 at 254 nm, at a flow |
| Method 10 | rate of 1.0 mL/min. at 25° C. |
| Chiral HPLC | on CHIRALPAK ® IC-3 was using 0.46 × 5 cm, 3 μm column, |
| Analytical | (Hex:DCM = 3:1)(0.1% DEA):EtOH = 70:30 at 254 nm, at a flow rate |
| Method 11 | of 1.0 mL/min. at 25° C. |
| Chiral HPLC | on CHIRALPAK ® IC was using 4.6 × 150 mm column, x % |
| Analytical | Heptane (or hexane, or acetonitrile) in EtOH (or MeOH) with or |
| Method 12 | without 0.2% diethylamine at 254 nm, at a flow rate of 0.5 mL/min. |
| | at 25° C. |
| Chiral HPLC | on CHIRALPAK ® IE was using 4.6 × 150 mm column, x % DCM |
| Analytical | in MeOH with or without 0.2% diethylamine at 254 nm, at a flow |
| Method 13 | rate of 0.5 mL/min. at 25° C. |
| Chiral HPLC | on CHIRALPAK ® IG was using 4.6 × 150 mm column, x % DCM |
| Analytical | (or hexane) in MeOH (or EtOH) with or without 0.2% diethylamine |
| Method 14 | at 254 nm, at a flow rate of 0.5 mL/min. at 25° C. |

TABLE 4
Chiral SFC Analytical Method

| | |
|---|---|
| Chiral SFC | on CHIRALPAK ® IF-3 was using 3.0 × 50 mm, 3 μm column, |
| Analytical | CO₂/MeOH with 0.1% DEA, 10% B to 50% B in 4.0 min, hold 2.0 |
| Method 1 | min at 50% at 220 nm, at a flow rate of 2.0 mL/min. at 35° C. |

In the following experimental descriptions, the following abbreviations may be used:

| | |
|---|---|
| Abbreviation | |
| Ac₂O | acetic anhydride |
| ACN | acetonitrile |
| aq. | aqueous |
| BOC, tBOC, | tert-butoxycarbonyl |
| Boc | |
| brine | saturated aqueous solution of sodium chloride |
| CH₂Cl₂ or | methylene chloride or 1,2-dichloromethane |
| DCM | |
| CH₃CN or | acetonitrile |
| MeCN or ACN | |
| CPME | cyclopentyl methyl ether |
| cPrNH₂ | cyclopropylamine |
| Cs₂CO₃ | cesium carbonate |
| Cu(OTf)₂ | copper(II) trifluoromethansulfonate |
| CuBr₂ | copper(II) bromide |
| Cy or CyH | cyclohexane |
| DCE | 1,2-dichloroethane |
| DCM | dichloromethane |
| DIAD | diisopropyl azodicarboxylate |
| DIBAL or | diisobutylaluminium hydride |
| DIBAL-H | |
| DIEA or | diisopropyl ethylamine |
| DIPEA | |
| DMAP | 4-dimethylaminopyidine |
| DMF | N,N-dimethylformamide |
| DMSO | dimethylsulfoxide |
| Et | ethyl |
| Et₃N or TEA | triethylamine |
| Et₂O | diethyl ether |
| EtOH | ethanol |
| EA or EtOAc | ethyl acetate |
| h, hr | hour(s) |
| HATU | O-(7-azabenzotriazol-lyl)-N,N,N′,N′-tetramethylyronium |
| | hexafluorophosphate |
| HCl | hydrochloric acid |
| HFIP | 1,1,1,3,3,3-hexafluoro-2-propanol |
| i-Pr₂Net | N′,N′-diisopropylethylamine |
| iPr₂O | diisopropyl ether |
| KOH | potassium hydroxide |
| Kot-Bu | potassium tert-butoxide |
| LCMS | liquid chromatography-mass spectroscopy |
| LiBH₄ | lithium borohydride |
| LiHDMS | lithium hexamethyldisilazide |
| LiOH | lithium hydroxide |
| mCPBA | 3-chloroperbenzoic acid |
| Me | methyl |
| MeCN | acetonitrile |
| MeOH or | methanol |
| CH₃OH | |
| min | minute (s) |
| MS | mass spectrum |
| NaBH₄ | sodium borohydride |
| nBuLi | n-butyllithium |
| NaBH₄ | sodium borohydride |
| Na₂CO₃ | sodium carbonate |
| NaH | sodium hydride |
| NaHCO₃ | sodium bicarbonate |
| NaOH | sodium hydroxide |
| Na₂SO₄ | sodium sulfate |
| NH₄Cl | ammonium chloride |
| NH₄OH | ammonium hydroxide |
| PCl₅ | phosphorus pentachloride |
| PE | petroleum ether |
| Ph | phenyl |
| PPh₃ | triphenyl phospine |
| POCl₃ | phosphoryl chloride |
| Pr | propyl |
| PyBroP ® | bromotripyrrolidinophosphonium |
| | hexafluorophosphate |
| rt | room temperature |
| t-BuOH | tert-butanol |
| TEA | triethylamine |
| TFA | trifluoroacetic acid |
| THF | tetrahydrofuran |
| UPLC | ultra performance liquid chromatography |
| UV | ultraviolet |

### Intermediates

### Intermediate 1

### 3-(3,5-difluorophenyl)isoxazolidine

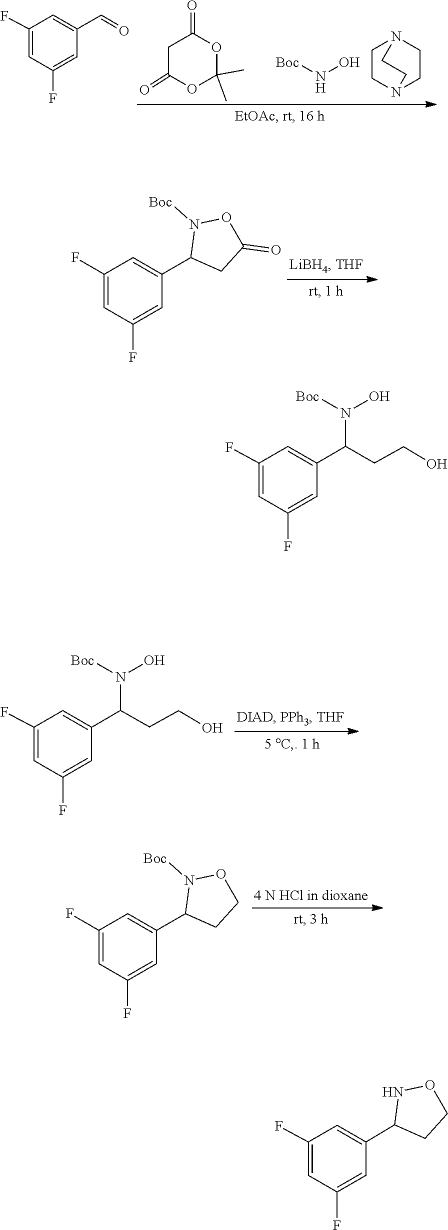

Step 1:

To a stirred solution of 3,5-difluorobenzaldehyde (1.42 g, 10 mmol) in EA (20 mL) were added 2,2-dimethyl-1,3-dioxane-4,6-dione (1.44 g, 10 mmol), 1,4-diazabicyclo[2.2.2]octane (112 mg, 1 mmol), and tert-butyl N-hydroxycarbamate (1.33 g, 10 mmol). The reaction mixture was stirred at rt for 16 hours, diluted with H₂O (50 mL), and extracted with EA (3×30 mL). The combined organic layers were washed with brine (100 mL), dried over Na₂SO₄, filtered, and concentrated under vacuum to afford tert-butyl 3-(3,5-difluorophenyl)-5-oxoisoxazolidine-2-carboxylate (1.3 g crude) as a yellow oil. LCMS (m/z) no mass signal, retention time: 1.083 min, LC/MS Method 6.

Step 2:

LiBH₄ (284 mg, 12.9 mmol) was added into a stirred mixture of tert-butyl 3-(3,5-difluorophenyl)-5-oxoisoxazolidine-2-carboxylate (1.3 g, 4.3 mmol) in THF (30 mL) in portions. The reaction mixture was stirred at rt for 1 hour, quenched by the addition of H₂O (50 mL), and extracted with EA (3×50 mL). The combined organic layers were washed with brine (100 mL), dried over anhydrous Na₂SO₄, filtered, and concentrated under vacuum to afford tert-butyl (1-(3,5-difluorophenyl)-3-hydroxypropyl)(hydroxy)carbamate (1.3 g crude) as a light yellow solid. LCMS (m/z) 204 (M+H−100)+, retention time: 1.390 min, LC/MS Method 5.

Step 3:

DIAD (1.3 g, 6.5 mmol) was added into a stirred mixture of tert-butyl(1-(3,5-difluorophenyl)-3-hydroxypropyl)(hydroxy)carbamate (1.3 g, 4.3 mmol), and PPh₃ (1.7 g, 6.5 mmol) in THF (30 mL) dropwise under nitrogen atmosphere at 5° C. The resulting mixture was stirred at 5° C. for 1 hour, quenched by the addition of H₂O (50 mL), and extracted with EA (3×50 mL). The combined organic layers were washed with brine (50 mL), dried over anhydrous Na₂SO₄, filtered, and concentrated under vacuum to afford tert-butyl 3-(3,5-difluorophenyl)isoxazolidine-2-carboxylate (2.2 g crude) as a yellow oil. LCMS (m/z) 186 (M+H−100)⁺, retention time: 1.540 min, LC/MS Method 5.

Step 4:

Tert-butyl 3-(3,5-difluorophenyl)isoxazolidine-2-carboxylate (2.2 g, 7.7 mmol) was added to a solution of HCl in dioxane (4 N, 30 mL). The resulting mixture was stirred at rt for 3 hours and concentrated under vacuum. The residue was diluted with H₂O (20 mL). The resulting mixture was adjusted to PH=8-9 with aqueous NaHCO₃ (10%, 100 mL), and extracted with DCM (3×50 mL). The combined organic layers were washed with brine (50 mL), dried over Na₂SO₄, filtered, and concentrated under vacuum. The residue was purified by column chromatography (EA in PE from 0% to 40%) to afford 3-(3,5-difluorophenyl)isoxazolidine (1.1 g crude) as a yellow solid. LCMS (m/z) 186 (M+H)⁺, retention time: 1.306 min, LC/MS Method 5.

### Intermediate 2

### (3-(3,5-difluorophenyl)isoxazolidin-2-yl)(piperidin-4-yl)methanone hydrochloride

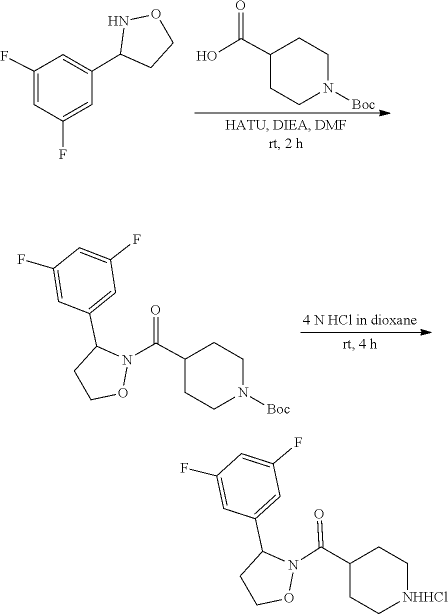

Step 1:

3-(3,5-Difluorophenyl)isoxazolidine (700 mg, 3.78 mmol) was added to a stirred mixture of 1-(tert-butoxycarbonyl)piperidine-4-carboxylic acid (866 mg, 3.78 mmol), HATU (1.44 g, 3.78 mmol), and DIEA (1.46 g, 11.3 mmol) in DMF (20 mL). The resulting mixture was stirred for 2 hours at rt, quenched by the addition of H₂O (50 mL), and extracted with DCM (3×50 mL). The combined organic layers were washed with brine (50 mL), dried over Na₂SO₄, filtered, and concentrated under vacuum. The residue was purified by column chromatography (EA in PE from 0% to 50%) to afford tert-butyl 4-(3-(3,5-difluorophenyl) isoxazolidine-2-carbonyl)piperidine-1-carboxylate (1.2 g, 80% yield) as a yellow solid. LCMS (m/z) 341 (M+H−56)⁺, retention time: 1.216 min, LC/MS Method 4.

Step 2:

Tert-butyl 4-(3-(3,5-difluorophenyl)isoxazolidine-2-carbonyl)piperidine-1-carboxylate (1.2 g, 3.0 mmol) was added to a solution of HCl in dioxane (4 N, 20 mL). The resulting mixture was stirred at rt for 4 hours and concentrated under vacuum to afford (3-(3,5-difluorophenyl)isoxazolidin-2-yl)(piperidin-4-yl)methanone hydrochloride (900 mg crude) as a yellow solid. LCMS (m/z) 297 (M+H−36)⁺, retention time: 0.699 min, LC/MS Method 4.

### Intermediate 3

### cis-(3-(3,5-difluorophenyl)isoxazolidin-2-yl)(3-fluoropiperidin-4-yl)methanone hydrochloride

### and

### trans-(3-(3,5-difluorophenyl)isoxazolidin-2-yl)(3-fluoropiperidin-4-yl)methanone hydrochloride

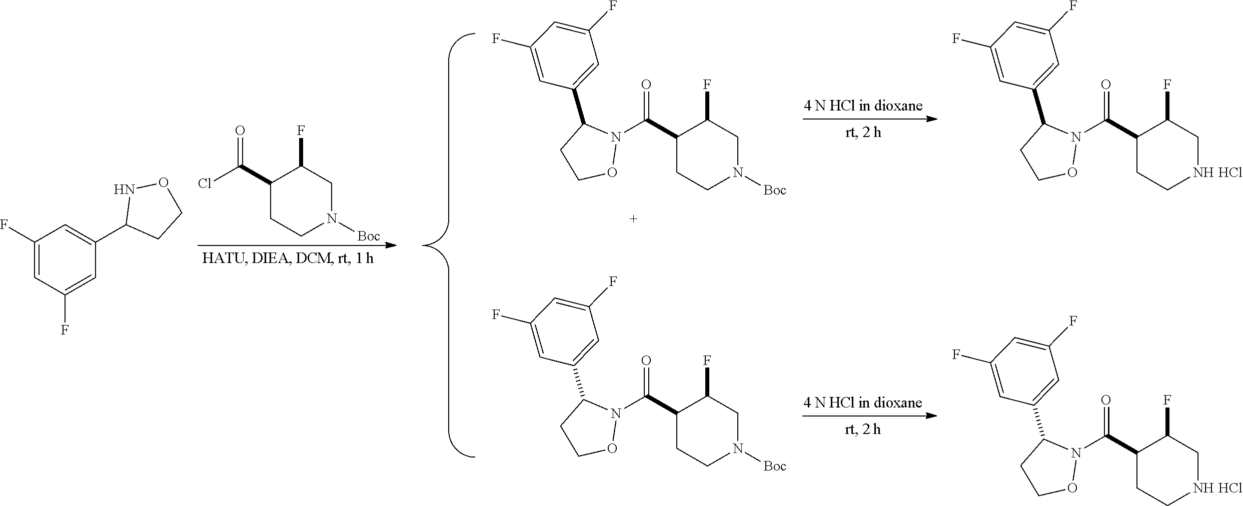

Step 1:

3-(3,5-Difluorophenyl)isoxazolidine (7.5 g, 40.5 mmol) was added to a stirred mixture of cis-1-(tert-butoxycarbonyl)-3-fluoropiperidine-4-carboxylic acid (10 g, 40.5 mmol), HATU (15.4 g, 121.5 mmol), and DIEA (15.7 g, 121.5 mmol) in DMF (60 mL). The resulting mixture was stirred for 1 hour at rt, quenched by the addition of H₂O (100 mL), and extracted with DCM (3×100 mL). The combined organic layers were washed with brine (50 mL), dried over Na₂SO₄, filtered, and concentrated under vacuum. The residue was purified by column chromatography (EA in PE from 0% to 80%) to afford cis-tert-butyl 4-(3-(3,5-difluorophenyl)isoxazolidine-2-carbonyl)-3-fluoropiperidine-1-carboxylate (2.8, 33% yield) as a white solid, LCMS (m/z) 359 (M+H−56)⁺, retention time: 1.202 min, LC/MS Method 22 and trans-tert-butyl 4-(3-(3,5-difluorophenyl)isoxazolidine-2-carbonyl)-3-fluoropiperidine-1-carboxylate (2.7, 32% yield) as a white solid. LCMS (m/z) 359 (M+H−56)⁺, retention time: 1.137 min, LC/MS Method 20.

Step 2:

cis-tert-butyl 4-(3-(3,5-difluorophenyl)isoxazolidine-2-carbonyl)-3-fluoropiperidine-1-carboxylate (2.8 g, 6.76 mmol) was added to a solution of HCl in dioxane (4 N, 60 mL). The resulting mixture was stirred at rt for 2 hours and concentrated under vacuum to afford cis-(3-(3,5-difluorophenyl)isoxazolidin-2-yl)(3-fluoropiperidin-4-yl)methanone hydrochloride (2.68 g crude) as a yellow solid. LCMS (m/z) 315 (M+H−37)⁺, retention time: 0.655 min, LC/MS Method 20.

Step 3:

trans-tert-butyl 4-(3-(3,5-difluorophenyl)isoxazolidine-2-carbonyl)-3-fluoropiperidine-1-carboxylate (2.7 g, 6.52 mmol) was added to a solution of HCl in dioxane (4 N, 60 mL). The resulting mixture was stirred at rt for 2 hours and concentrated under vacuum to afford trans-(3-(3,5-difluorophenyl)isoxazolidin-2-yl)(3-fluoropiperidin-4-yl)methanone hydrochloride (2.2 g crude) as a white solid. LCMS (m/z) 315 (M+H−37)⁺, retention time: 0.905 min, LC/MS Method 13.

### Intermediate 4

### 2-bromo-5-methyl-1,3,4-oxadiazole

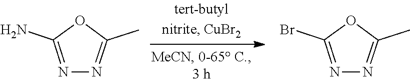

Tert-butyl nitrite (4.7 g, 40.4 mmol) was added into a stirred mixture of 5-methyl-1,3,4-oxadiazol-2-amine (2 g, 20.2 mmol) and CuBr₂ (6.7 g, 30.3 mmol) in MeCN at 0° C. under nitrogen atmosphere. The resulting mixture was heated to 65° C. and stirred for 3 hours. After cooled to rt, the reaction mixture was diluted with H₂O (100 mL), and extracted with EA (3×100 mL). The combined organic layers were washed with brine (200 mL), dried over anhydrous Na₂SO₄, and concentrated under vacuum. The residue was purified by column chromatography (EA in PE from 0% to 50%) to afford 2-bromo-5-methyl-1,3,4-oxadiazole (1 g, 31% yield) as a yellow solid. LCMS (m/z) 163 (M+H⁺), retention time: 0.390 min. LC/MS Method 13.

### Intermediate 5

### 6-chloro-N-methylpyrimidine-4-carboxamide

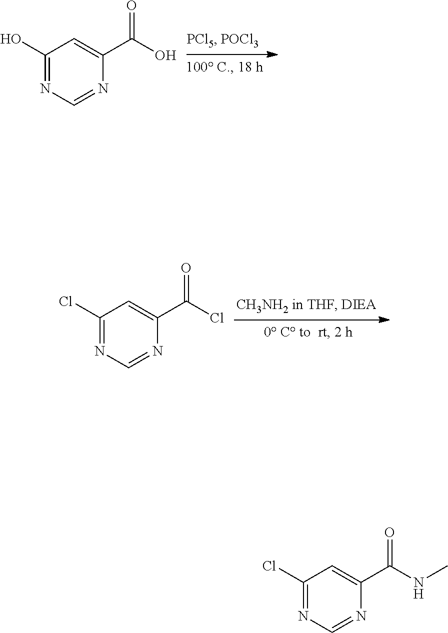

Step 1:

To a solution of 6-hydroxypyrimidine-4-carboxylic acid (4.5 g, 32.1 mmol) in POCl₃ (80 mL) was added PCl₅ (6.6 g, 32.1 mmol). The resulting mixture was heated to 100° C. and stirred for 18 hours. After cooled to rt, the reaction mixture was concentrated under vacuum to afford 6-chloropyrimidine-4-carbonyl chloride (15.0 g crude) as a black oil. LCMS (m/z) 169 (M+H⁺), retention time: 0.532 min. LC/MS Method 14.

Step 2:

A solution of methanamine in THF (2 M in THF, 6.7 mL, 13.4 mmol) was added to a solution of 6-chloropyrimidine-4-carbonyl chloride (2.3 g, 13.1 mmol) in DCM (40 mL) at 0° C. under N₂ atmosphere, followed by addition of DIEA (6.3 g, 49.1 mmol). The resulting mixture was stirred at 0° C. for an hour then stirred at rt for another hour. The resulting mixture was concentrated under vacuum. The residue was purified by column chromatography (EA in PE from 0% to 30%) to give the title compound (2.0 g, 89% yield) as a yellow solid. LCMS (m/z) 172 (M+H)⁺, retention time: 0.487 min. LC/MS Method 14.

### Intermediate 6

### 6-chloro-N,N-dimethylpyrimidine-4-carboxamide

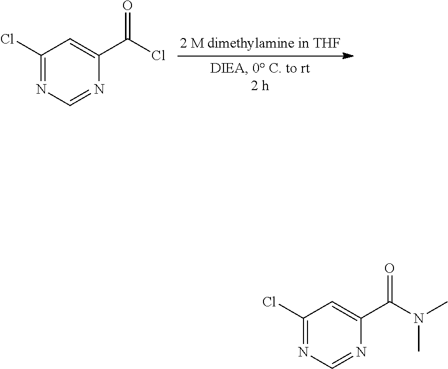

A solution of dimethylamine in THF (2 M in THF, 6.4 mL, 12.8 mmol) was added to a solution of 6-chloropyrimidine-4-carbonyl chloride (2.2 g, 12.5 mmol) in DCM (40 mL), followed by addition of DIEA (6.0 g, 46.8 mmol) at 0° C. under nitrogen atmosphere. The resulting mixture was stirred at 0° C. for an hour and rt for another hour. The mixture was concentrated under vacuum. The residue was purified by column chromatography (EA in PE from 0% to 30%) to give 6-chloro-N,N-dimethylpyrimidine-4-carboxamide (2.0 g, 86% yield) as a yellow solid. LCMS (m/z) 186 (M+H)⁺, retention time: 0.457 min, LC/MS Method 14.

### Intermediate 7

### 2-chloro-5-(methylsulfonyl)pyrimidine

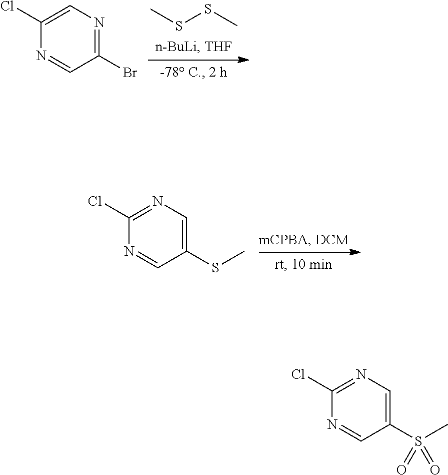

Step 1:

A solution of n-BuLi in hexane (2.5 M, 4 mL, 10 mmol) was added to a stirred mixture of 5-bromo-2-chloropyrimidine (1.93 g, 10 mmol) and 1,2-dimethyldisulfane (1.13 g, 12 mmol) in THF (50 mL) dropwise at −78° C. The resulting mixture was stirred for 2 hours at −78° C., quenched by the addition of saturated aqueous NH₄Cl (50 mL), and extracted with EA (3×50 mL). The combined organic layers were washed with brine (50 mL), dried over anhydrous Na₂SO₄, filtered, and concentrated under vacuum. The residue was purified by column chromatograph (EA in PE from 0% to 7%) to afford 2-chloro-5-(methylthio) pyrimidine (380 mg, 24% yield) as a white solid. LCMS (m/z) 161 (M+H)⁺, retention time: 0.79 min, LC/MS Method 9.

Step 2:

m-CPBA (545 mg, 3.15 mmol) was added to a stirred mixture of 2-chloro-5-(methylthio)pyrimidine (340 mg, 2.1 mmol) in DCM (20 mL). The resulting mixture was stirred for 10 minutes at rt, quenched by the addition of aqueous NaHCO₃ (10%, 40 mL), and extracted with DCM (3×50 mL). The combined organic layers was washed with brine (50 mL), dried over anhydrous Na₂SO₄, filtered, and concentrated under vacuum. The residue was purified by column chromatograph (EA in PE from 0% to 30%) to afford 2-chloro-5-(methylsulfonyl)pyrimidine (120 mg, 30% yield) as a white solid. LCMS (m/z) 193 (M+H)⁺, retention time: 0.56 min, LC/MS Method 9.

### Intermediate 8

### 4-chloro-6-(methylsulfinyl)pyrimidine

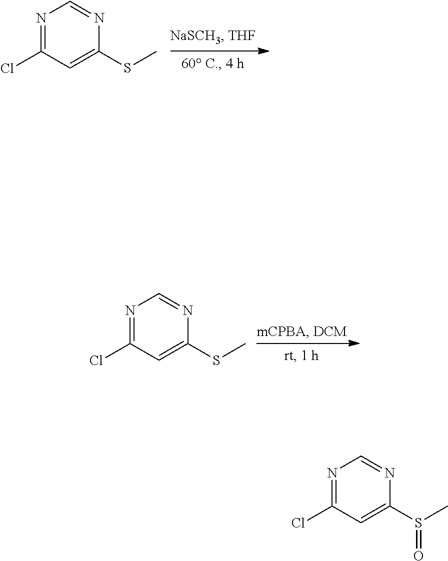

Step 1:

NaSCH₃ (4.6 g, 66 mmol) was added to a mixture of 4-chloro-6-(methylthio) pyrimidine (9.8 g, 66 mmol) in THF (60 mL). The resulting mixture was stirred for 4 hours at 60° C., quenched by the addition of H₂O (50 mL), and extracted with EA (3×50 mL). The combined organic layers were washed with brine (50 mL), dried over anhydrous Na₂SO₄, filtered, and concentrated under vacuum to afford 4-chloro-6-(methylthio)pyrimidine (8.2 g crude) as a yellow oil. LCMS (m/z) 161 (M+H)⁺, retention time: 0.857 min, LC/MS Method 20.

Step 2:

mCPBA (5.6 g, 32.6 mmol) was added to a stirred mixture of 4-chloro-6-(methylthio)pyrimidine (3.5 g, 21.7 mmol) in DCM (100 mL). The resulting mixture was stirred for 1 hour at rt, quenched by the addition of aqueous NaHCO₃ (10%, 100 mL), and extracted with DCM (3×50 mL). The combined organic layers were washed with brine (50 mL), dried over anhydrous Na₂SO₄, filtered, and concentrated under vacuum. The residue was purified by column chromatograph (EA in PE from 0% to 20%) to afford 4-chloro-6-(methylsulfinyl)pyrimidine (2.8 g, 73% yield) as a yellow solid. LCMS (m/z) 177 (M+H)⁺, retention time: 0.345 min, LC/MS Method 20.

### EXAMPLES

### Example 1 and Example 2

### (R)-1-(4-(3-(3,5-difluorophenyl)isoxazolidine-2-carbonyl)piperidin-1-yl)ethan-1-one

### and

### (S)-1-(4-(3-(3,5-difluorophenyl)isoxazolidine-2-carbonyl)piperidin-1-yl)ethan-1-one

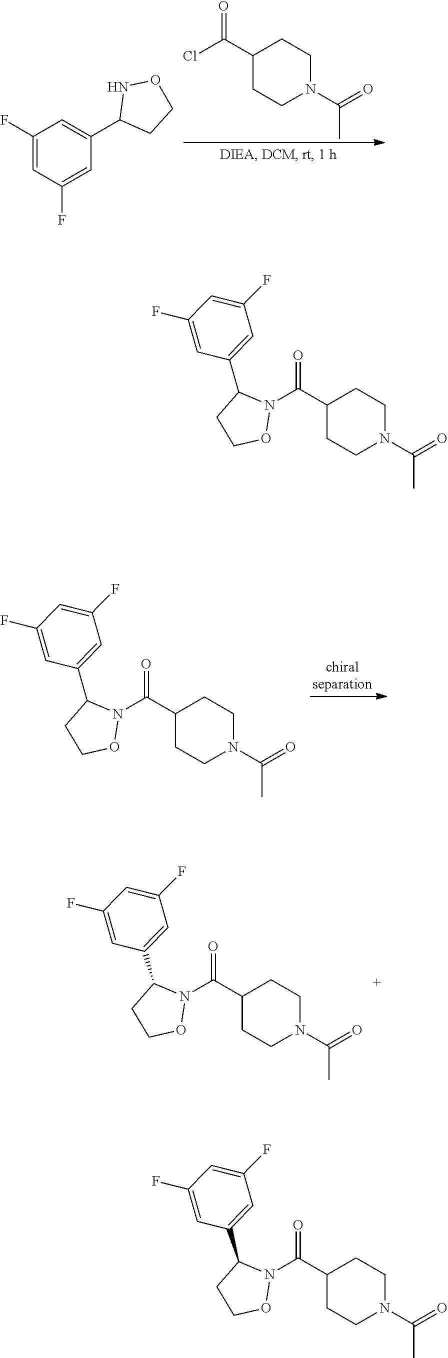

Step 1:

1-Acetylpiperidine-4-carbonyl chloride (338 mg, 1.78 mmol) was added to a stirred mixture of 3-(3,5-difluorophenyl)isoxazolidine (300 mg, 1.62 mmol), and DIEA (627 mg, 4.86 mmol) in DCM (20 mL). The resulting mixture was stirred for 1 hour at rt, quenched by the addition of H₂O (60 mL), and extracted with DCM (3×20 mL). The combined organic layers were washed with brine (40 mL), dried over anhydrous Na₂SO₄, filtered, and concentrated under vacuum. The residue was purified by column chromatograph (EA in PE from 0% to 100%) to afford 1-(4-(3-(3,5-difluorophenyl)isoxazolidine-2-carbonyl)piperidin-1-yl)ethan-1-one (280 mg, 52% yield) as a yellow oil. LCMS (m/z) 339 (M+H)⁺, retention time: 2.040 min, LC/MS Method 10.

Step 2:

The racemate of 1-(4-(3-(3,5-difluorophenyl)isoxazolidine-2-carbonyl)piperidin-1-yl)ethan-1-one (280 mg, 0.83 mmol) were separated by Prep-Chiral-SFC with the following conditions: Column: CHIRALPAK IF, 2×25 cm, 5 um; Mobile Phase A: CO₂: 65%, Mobile Phase B: MeOH: 35%; Flow rate: 40 mL/min; UV 220 nm; Rt1: 3.95 min; Rt2: 5.55 min to afford two title compounds:

Example 1: (R)-1-(4-(3-(3,5-difluorophenyl)isoxazolidine-2-carbonyl)piperidin-1-yl)ethan-1-one was obtained as a yellow oil (87.3 mg, 62% yield). LCMS (m/z) 339 (M+H)⁺, retention time: 1.077 min, LC/MS Method 7. Anal-SFC Method 1, retention time: 1.618 min, % ee=100.0%. ¹H NMR (400 MHz, CD₃OD-d₄) ppm 6.98-6.80 (m, 3H), 5.36 (t, J=7.6 Hz, 1H), 4.50-4.40 (m, 1H), 4.35-4.25 (m, 1H), 4.02-3.86 (m, 2H), 3.29-3.00 (m, 2H), 2.99-2.87 (m, 1H), 2.82-2.69 (m, 1H), 2.33-2.20 (m, 1H), 2.09 (s, 3H), 2.02-1.88 (m, 1H), 1.90-1.72 (m, 1H), 1.70-1.48 (m, 2H).

Example 2: (S)-1-(4-(3-(3,5-difluorophenyl)isoxazolidine-2-carbonyl)piperidin-1-yl)ethan-1-one was obtained as a yellow oil (95.7 mg, 68% yield). LCMS (m/z) 339 (M+H)⁺, retention time: 1.077 min, LC/MS Method 7. Anal-SFC Method 1, retention time: 2.246 min, % ee=99.4%. ¹H NMR (400 MHz, CD₃OD-d₄) ppm 6.96-6.80 (m, 3H), 5.34 (t, J=7.6 Hz, 1H), 4.52-4.40 (m, 1H), 4.34-4.26 (m, 1H), 4.00-3.88 (m, 2H), 3.28-3.00 (m, 2H), 3.00-2.86 (m, 1H), 2.84-2.68 (m, 1H), 2.34-2.20 (m, 1H), 2.09 (s, 3H), 2.02-1.88 (m, 1H), 1.90-1.70 (m, 1H), 1.70-1.48 (m, 2H).

### Example 3 and Example 4

### (R)-6-(4-(3-(3,5-difluorophenyl)isoxazolidine-2-carbonyl)piperidin-1-yl)pyrimidine-4-carboxamide and

### (S)-6-(4-(3-(3,5-difluorophenyl)isoxazolidine-2-carbonyl)piperidin-1-yl)pyrimidine-4-carboxamide

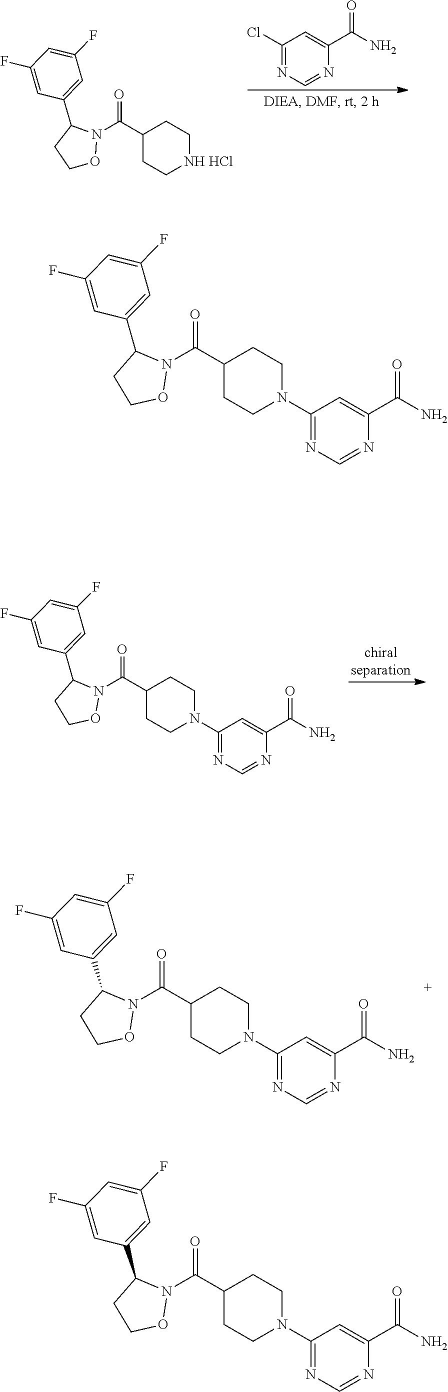

Step 1:

DIEA (522 mg, 4.05 mmol) was added to a mixture of (3-(3,5-difluorophenyl)isoxazolidin-2-yl)(piperidin-4-yl)methanone hydrochloride (450 mg, 1.35 mmol), and 6-chloropyrimidine-4-carboxamide (213 mg, 1.35 mmol) in DMF (10 mL). The resulting mixture was stirred for 2 hours at rt, quenched by the addition of H₂O (60 mL), and extracted with DCM (3×20 mL). The combined organic layers were washed with brine (40 mL), dried over anhydrous Na₂SO₄, filtered, and concentrated under vacuum. The residue was purified by column chromatography (EA in PE from 0% to 100%) to afford 6-(4-(3-(3,5-difluorophenyl) isoxazolidine-2-carbonyl)piperidin-1-yl)pyrimidine-4-carboxamide (370 mg, 66% yield) as a yellow solid. LCMS (m/z) 418 (M+H)⁺, retention time: 0.929 min, LC/MS Method 2.

Step 2:

The racemate of 6-(4-(3-(3,5-difluorophenyl)isoxazolidine-2-carbonyl)piperidin-1-yl) pyrimidine-4-carboxamide (370 mg, 0.89 mmol) were separated by Prep-Chiral-HPLC with the following conditions: Column: CHIRALPAK IC, 2×25 cm, 5 um; Mobile Phase A: Hexane, Mobile Phase B: EtOH; Flow rate: 15 mL/min; Gradient: 50 B to 50 B in 18 min; UV 254 & 220 nm; Rt1: 11.88 min; Rt2: 14.48 min to afford two title compounds:

Example 3: (R)-6-(4-(3-(3,5-difluorophenyl)isoxazolidine-2-carbonyl)piperidin-1-yl)pyrimidine-4-carboxamide was obtained as a white solid (125.2 mg, 68% yield). LCMS (m/z) 418 (M+H)⁺, retention time: 1.346 min, LC/MS Method 1. Chiral-HPLC Method 2, retention time: 2.246 min, % ee=100.0%. ¹H NMR (300 MHz, CD₃OD-d₄) ppm 8.51 (s, 1H), 7.37 (s, 1H), 6.98-6.80 (m, 3H), 5.42-5.30 (m, 1H), 4.65-4.45 (m, 2H), 4.40-4.28 (m, 1H), 4.06-3.92 (m, 1H), 3.28-3.02 (m, 3H), 2.99-2.88 (m, 1H), 2.36-2.26 (m, 1H), 2.12-2.00 (m, 1H), 1.98-1.60 (m, 3H).

Example 4: (S)-6-(4-(3-(3,5-difluorophenyl)isoxazolidine-2-carbonyl)piperidin-1-yl)pyrimidine-4-carboxamide was obtained as a white solid (113 mg, 61% yield). LCMS (m/z) 418 (M+H)⁺, retention time: 1.346 min, LC/MS Method 1. Chiral-HPLC Method 2, retention time: 2.653 min, % ee=95.0%. ¹H NMR (300 MHz, CD₃OD-d₄) ppm 8.51 (s, 1H), 7.38 (s, 1H), 6.98-6.80 (m, 3H), 5.40-5.30 (m, 1H), 4.62-4.40 (m, 2H), 4.38-4.28 (m, 1H), 4.06-3.92 (m, 1H), 3.28-3.02 (m, 3H), 2.99-2.88 (m, 1H), 2.36-2.24 (m, 1H), 2.12-2.00 (m, 1H), 1.98-1.58 (m, 3H).

The following compounds were synthesized in an analogous manner. Example 5 and Example 6 were synthesized using the same condition. For Examples 7 and 8, the displacement was carried out using NaHCO₃ as a base and DMF as the solvent while heating at 80° C. for 3 hours. **

| | | | | Chiral | | |
|---|---|---|---|---|---|---|
| | | | | HPLC/SFC: | LC: | |
| | | | | retention | retention | |
| | | | | time (min); | Time | |
| | | | | Chiral | (min); | |
| | | | | HPLC/SFC | LC/MS | |
| | | | | Analytic | Method of | MS |
| Ex. | Name | Structure | ¹H NMR | Method | analysis | (M + H)⁺ |
| 5 | (S)-(3-(3,5- difluorophenyl) isoxazolidin-2- yl)(1-(5-methyl- 1,3,4-oxadiazol- 2-yl)piperidin-4- yl)methanone | 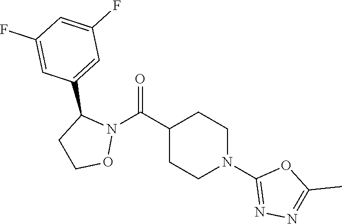 [SMILES: Cc1nnc(N2CCC(C(=O)N3OCC[C@H]3c3cc(F)cc(F)c3)CC2)o1] | ¹H NMR (300 MHz, CD₃OD-d₄) ppm 7.00-6.80 (m, 3H), 5.37 (dd, J = 8.4, 6.6 Hz, 1H), 4.36-4.23 (m, 1H), 4.05-3.88 (m, 3H), 3.22-3.00 (m, 3H), 3.00-2.88 (m, 1H), 2.37 (s, 3H), 2.32-2.23 (m, 1H), 2.08-1.98 (m, 1H), 1.90-1.66 (m, 3H) | 2.154 min, Chiral HPLC Method 1 | 1.321 min, LC/MS Method 1 | 379.2 |
| 6 | (R)-(3-(3,5- difluorophenyl) isoxazolidin-2- yl)(1-(5-methyl- 1,3,4-oxadiazol- 2-yl)piperidin-4- yl)methanone | 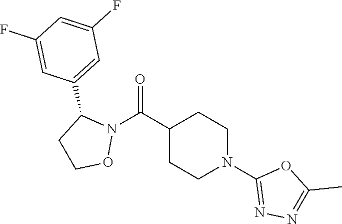 [SMILES: Cc1nnc(N2CCC(C(=O)N3OCC[C@@H]3c3cc(F)cc(F)c3)CC2)o1] | ¹H NMR (300 MHz, CD₃OD-d₄) ppm 7.00-6.80 (m, 3H), 5.37 (dd, J = 8.4, 6.3 Hz, 1H), 4.38-4.28 (m, 1H), 4.02-3.88 (m, 3H), 3.22-3.00 (m, 3H), 3.00-2.88 (m, 1H), 2.40-2.22 (m, 4H), 2.08-1.96 (m, 1H), 1.90-1.64 (m, 3H) | 2.623 min, Chiral HPLC Method 1 | 1.133 min, LC/MS Method 7 | 379.2 |
| 7 | (R)-6-(4-(3-(3,5- difluorophenyl) isoxazolidine-2- carbonyl) piperidin-1- yl)pyrimidine-4- carbonitrile | 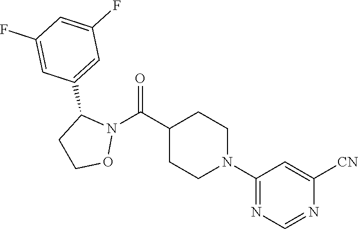 [SMILES: N#Cc1cc(N2CCC(C(=O)N3OCC[C@@H]3c3cc(F)cc(F)c3)CC2)ncn1] | ¹H NMR (400 MHz, CDCl₃-d) ppm 8.59 (s, 1H), 6.88-6.76 (m, 3H), 6.76-6.68 (m, 1H), 5.35 (dd, J = 8.8, 6.4 Hz, 1H), 4.60-4.26 (m, 3H), 3.98-3.88 (m, 1H), 3.26-3.06 (m, 3H), 2.92-2.80 (m, 1H), 2.40-2.28 (m, 1H), 2.12-2.04 (m, 1H), 1.90-1.70 (m, 3H) | 1.540 min, Chiral HPLC Method 3 | 1.703 min, LC/MS Method 3 | 400.2 |
| 8 | (S)-6-(4-(3-(3,5- difluorophenyl) isoxazolidine-2- carbonyl) piperidin-1- yl)pyrimidine-4- carbonitrile | 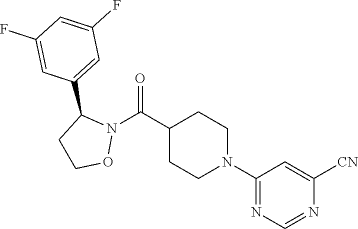 [SMILES: N#Cc1cc(N2CCC(C(=O)N3OCC[C@H]3c3cc(F)cc(F)c3)CC2)ncn1] | ¹H NMR (400 MHz, CDCl₃-d) ppm 8.59 (s, 1H), 6.88-6.76 (m, 3H), 6.76-6.66 (m, 1H), 5.36 (dd, J = 8.8, 6.0 Hz, 1H), 4.70-4.02 (m, 3H), 3.96-3.88 (m, 1H), 3.26-3.06 (m, 3H), 2.92-2.80 (m, 1H), 2.40-2.28 (m, 1H), 2.12-2.04 (m, 1H), 1.90-1.68 (m, 3H). | 3.416 min, Chiral HPLC Method 3 | 1.703 min, LC/MS Method 3 | 400.2 |

Alternatively, compounds of the preceding type can be prepared as single enantiomers without utilization of chiral chromatography. For instance, Example 5 can be prepared according to the conditions outlined below.

### Alternate Synthesis of

### Example 5

### (S)-(3-(3,5-difluorophenyl)isoxazolidin-2-yl)(1-(5-methyl-1,3,4-oxadiazol-2-yl)piperidin-4-yl)methanone

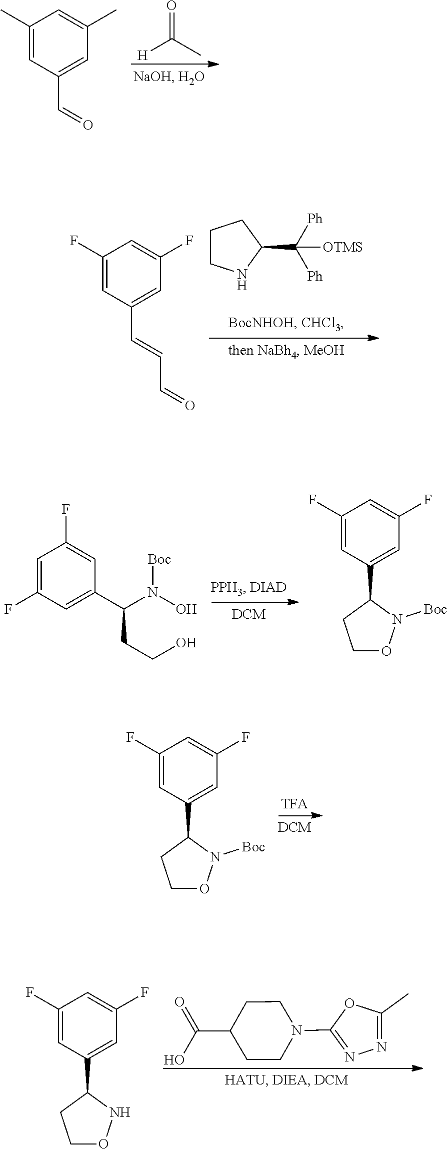

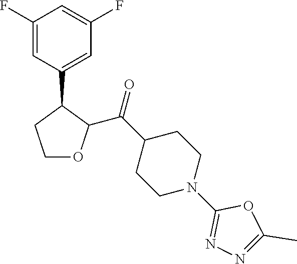 [SMILES: Cc1nnc(N2CCC(C(=O)C3OCC[C@H]3c3cc(F)cc(F)c3)CC2)o1]

Step 1

To a suspension of 3,5-difluorobenzaldehyde (286 g, 2.01 mol) in water (2.5 L) was added acetaldehyde (35% in water, 380 g, 3.01 mol) at 0° C. Then an aqueous solution of sodium hydroxide (1.0 M, 2.1 L, 2.11 mol) was added drop wise over 30 minutes. The mixture was stirred at rt for 48 hours. The resulting mixture was diluted with water (1.0 L), and the aqueous layer was extracted with EtOAc (2 L×3). Combined organic phases were washed with brine (2 L), dried over anhydrous sodium sulfate, and concentrated in vacuo. The resulting residue was purified by normal phase column chromatography (10% EtOAc in petroleum ether) to afford 3-(3,5-difluorophenyl)acrylaldehyde (230.0 g, 1367.9 mmol, 68% yield) as a pale yellow solid. ¹H NMR (300 MHz, CDCl₃) δ 9.74 (d, J=7.5 Hz, 1H), 7.40 (d, J=15.9 Hz, 1H), 7.13-7.06 (m, 2H), 6.94-6.88 (m, 1H), 6.73 (dd, J=7.2 Hz, 16.2 Hz, 1H).

Step 2

To a suspension of (S)-2-(diphenyl((trimethylsilyl)oxy)methyl)pyrrolidine (11.7 g, 35.8 mmol) in CHCl₃ (600 mL) was added 3-(3,5-difluorophenyl)acrylaldehyde (20.0 g, 119.1 mmol) and tert-butyl hydroxycarbamate (31.7 g, 238.2 mmol). The reaction mixture was stirred at 0° C. for 3 hours. Then MeOH (60 mL) was added to the mixture at 0° C., followed by NaBH₄ (9.1 g, 238.2 mmol) and the mixture was stirred at rt for 16 hours. Concentrated under reduced pressure, the residue was purified by silica gel column chromatography with PE/EA=7/1 to 2/1 to get the crude product as a yellow oil. ¹H NMR (300 MHz, CDCl₃) δ 7.47-7.36 (m, 1H), 6.98-6.91 (m, 2H), 6.75-6.69 (m, 1H), 5.17 (dd, J=5.1, 10.5 Hz, 1H), 3.86-3.73 (m, 2H), 2.76-2.67 (m, 1H), 2.40-2.30 (m, 1H), 2.07-1.98 (m, 1H), 1.44 (s, 9H); MS (m/z) 204.2 (M+H-Boc)⁺, retention time: 1.50 min, UPLC/MS Method 1 using 5% ACN. Chiral HPLC retention time: 7.446 min; Chiral HPLC Analytical Method 14, eluent: 90% (v) hexane+10% EtOH+0.2% (v) DEA.

Step 3

To a solution of (S)-tert-butyl (1-(3,5-difluorophenyl)-3-hydroxypropyl)(hydroxy)carbamate (3.7 g, 12.21 mmol) and triphenylphosphine (6.4 g, 24.42 mmol) in DCM (850 mL) was added a solution of DIAD (4.94 g, 24.42 mmol) in DCM (50 mL) dropwise at 0° C. under N₂. The mixture was stirred at rt for 16 hours. Concentrated, the residue was purified by silica column chromatography using 0-20% EtOAc in petroleum ether to afford (S)-tert-butyl 3-(3,5-difluorophenyl)isoxazolidine-2-carboxylate (2.0 g, 7.0 mmol, yield: 57%) as light yellow oil. ¹H NMR (300 MHz, CDCl₃) δ 6.92-6.85 (m, 2H), 6.72-6.64 (m, 1H), 5.17 (dd, J=7.2, 11.6 Hz, 1H), 4.19-4.12 (m, 2H), 3.90-3.82 (m, 1H), 2.83-2.73 (m, 1H), 2.30-2.18 (m, 1H), 1.47 (s, 9H); MS (m/z) 229.9 (M+H−56)⁺, retention time: 2.27 min, LC/MS Method 23 using 20% ACN.

Step 4

TFA (5 mL) was added slowly to a solution of (S)-tert-butyl 3-(3,5-difluorophenyl)isoxazolidine-2-carboxylate (2 g, 7.02 mmol) in DCM (20 mL) at 0° C. The mixture was stirred at rt for 2 hours. The mixture was concentrated to afford the crude (S)-3-(3,5-difluorophenyl)isoxazolidine as TFA salt (3 g, purity: 70%), which was used for the next step without other further purification. ¹H NMR (300 MHz, CDCl₃) δ 6.97-6.83 (m, 3H), 4.96-4.91 (m, 1H), 4.54-4.46 (m, 1H), 4.38-4.29 (m, 1H), 3.00-2.873 (m, 1H), 2.67-2.56 (m, 1H); MS (m/z) 186.3 (M+H)⁺, retention time: 1.35 min, UPLC/MS Method 1 using 5% ACN.

Step 5

A mixture of crude (S)-3-(3,5-difluorophenyl)isoxazolidine TFA salt (3.0 g, 7.02 mmol, purity: 70%), 1-(5-methyl-1,3,4-oxadiazol-2-yl)piperidine-4-carboxylic acid (1.78 g, 8.42 mmol), HATU (4.0 g, 10.53 mmol) and DIEA (4.56 g, 35.1 mmol) in DCM (50 mL) was stirred at rt for 16 hours. After completion, 50 mL water was added. The organic phase was washed with saturated aqueous NH₄Cl solution (60 mL×3), dried over anhydrous Na₂SO₄. The filtrate was concentrated, the residue was purified by C18 (%5-95% ACN in H₂O) to afford the crude product, which was further purified by silica column chromatography using 0-8% MeOH in DCM to afford (S)-(3-(3,5-difluorophenyl)isoxazolidin-2-yl)(1-(5-methyl-1,3,4-oxadiazol-2-yl)piperidin-4-yl)methanone (1.8 g, 4.76 mmol, yield: 67%) as white solid. LCMS (m/z) 379.2 (M+H)⁺, retention time: 3.275 min (LCMS Method 25, 20%-70% ACN), ¹H NMR (400 MHz, CDCl₃) δ 6.82-6.80 (m, 2H), 6.72-6.67 (m, 1H), 5.36 (dd, J=6.0 Hz, 8.8 Hz, 1H), 4.30-4.25 (m, 1H), 4.03-3.87 (m, 3H), 3.15-3.03 (m, 2H), 2.99-2.93 (m, 1H), 2.89-2.81 (m, 1H), 2.38-2.28 (m, 4H), 2.06-1.90 (m, 1H), 1.89-1.80 (m, 3H).

Examples 2, 4, and 8 were also alternatively synthesized in an analogous manner.

| | | | | LC: | |
|---|---|---|---|---|---|
| | | | | retention | |
| | | | | Time (min); | |
| | | | | QC Method | |
| Exam- | | | | of analysis, | MS: |
| ple | Name | Structure | ¹H NMR | eluent | (M + H)⁺ |
| 2 | (S)-1-(4-(3-(3,5- difluorophenyl)isoxazolidine- 2-carbonyl)piperidin-1- yl)ethanone | 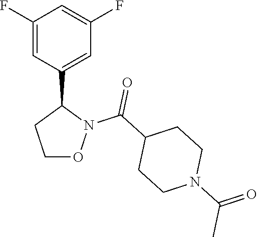 [SMILES: CC(=O)N1CCC(C(=O)N2OCC[C@H]2c2cc(F)cc(F)c2)CC1] | ¹H NMR (400 MHz, CDCl₃) δ 6.82-6.80 (m, 2H), 6.72-6.67 (m, 1H), 5.36 (t, J = 7.6 Hz, 1H), 4.60-4.51 (m, 1H), 4.29-4.25 (m, 1H), 3.93-3.84 (m, 2H), 3.17-3.13 (m, 1H), 3.02-2.98 (m, 1H), 2.86-2.73 (m, 1H), 2.35-2.30 (m, 1H), 2.09 (s, 3H), 2.02-1.93 (m, 1H), 1.76-1.68 (m, 3H). | 3.253 min; Method 24, 5% ACN | 339.2 |
| 4 | (S)-6-(4-(3-(3,5- difluorophenyl)isoxazolidine- 2-carbonyl)piperidin-1- yl)pyrimidine-4-carboxamide | 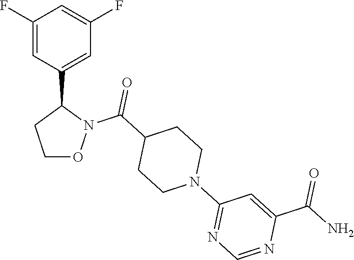 [SMILES: NC(=O)c1cc(N2CCC(C(=O)N3OCC[C@H]3c3cc(F)cc(F)c3)CC2)ncn1] | ¹H NMR (400 MHz, CDCl₃) δ 8.55 (s, 1H), 7.80 (s, 1H), 7.38 (s, 1H), 6.85-6.73 (m, 3H), 6.82-6.80 (m, 2H), 6.72-6.67 (m, 1H), 5.62 (s, 1H), 5.37 (t, J = 8 Hz, 1H), 4.52-4.49 (m, 2H), 4.31-4.26 (m, 1H), 3.95-3.88 (m, 1H), 3.19-3.07 (m, 3H), 2.87-2.83 (m, 1H), 2.36-2.30 (m, 1H), 2.08-2.04 (m, 1H), 1.86-1.77 (m, 3H). | 3.355 min; Method 25, 20%-70% ACN | 418.2 |
| 8 | (S)-6-(4-(3-(3,5- difluorophenyl)isoxazolidine- 2-carbonyl)piperidin-1- yl)pyrimidine-4-carbonitrile | 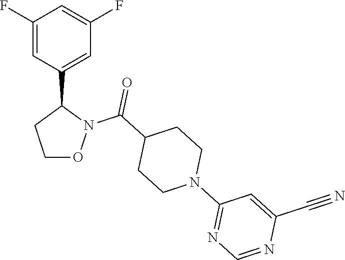 [SMILES: N#Cc1cc(N2CCC(C(=O)N3OCC[C@H]3c3cc(F)cc(F)c3)CC2)ncn1] | ¹H NMR (400 MHz, CDCl₃) δ 8.58 (s, 1H), 6.85-6.73 (m, 3H), 6.71-6.68 (m, 1H), 5.36 (dd, J = 6 Hz, 8.8 Hz, 1H), 4.32-4.27 (m, 3H), 3.95-3.89 (m, 1H), 3.24-3.09 (m, 3H), 2.89-2.85 (m, 1H), 3.37-2.31 (m, 1H), 2.11-2.07 (m, 1H), 1.87-1.78 (m, 3H). | 3.498 min; Method 25, 30%-70% ACN | 400.2 |

### Example 9

### (S)-6-(4-(3-(3,5-difluorophenyl)isoxazolidine-2-carbonyl)piperidin-1-yl)-N-methylpyrimidine-4-carboxamide

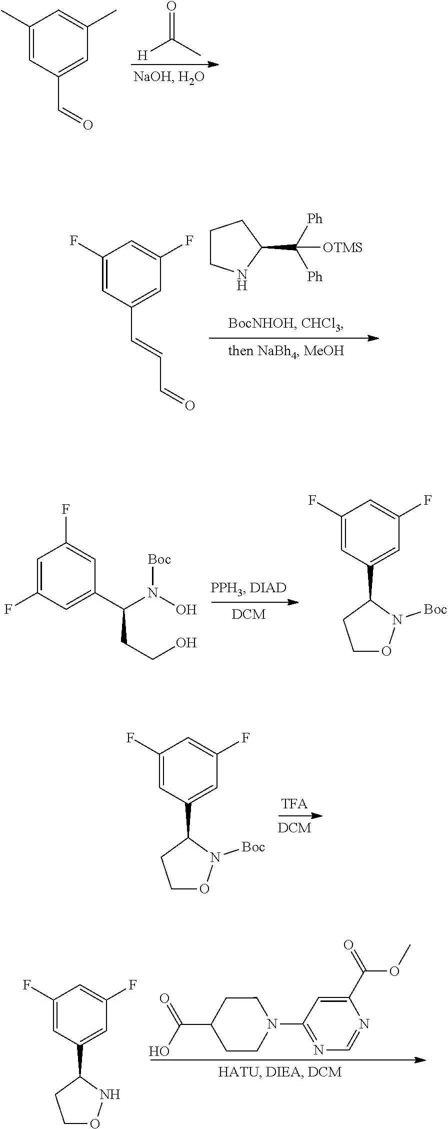

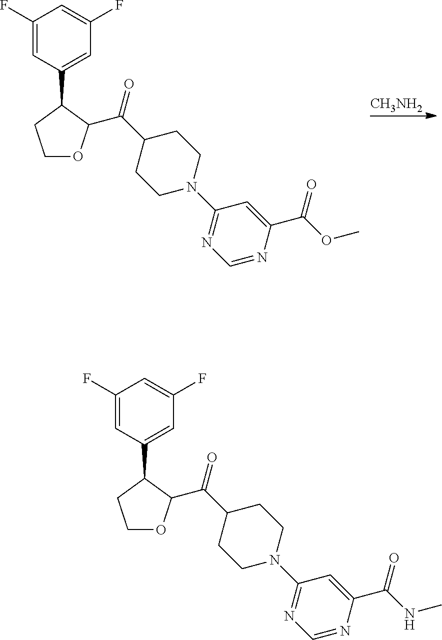 [SMILES: COC(=O)c1cc(N2CCC(C(=O)C3OCC[C@H]3c3cc(F)cc(F)c3)CC2)ncn1 ; "CH3NH2" ; CNC(=O)c1cc(N2CCC(C(=O)C3OCC[C@H]3c3cc(F)cc(F)c3)CC2)ncn1]

Step 1

To a suspension of 3,5-difluorobenzaldehyde (286 g, 2.01 mol) in water (2.5 L) was added acetaldehyde (35% in water, 380 g, 3.01 mol) at 0° C. Then an aqueous solution of sodium hydroxide (1.0 M, 2.1 L, 2.11 mol) was added drop wise over 30 minutes. The mixture was stirred at rt for 48 hours. The resulting mixture was diluted with water (1.0 L), and the aqueous layer was extracted with EtOAc (2 L×3). Combined organic phases were washed with brine (2 L), dried over anhydrous sodium sulfate, and concentrated in vacuo. The resulting residue was purified by normal phase column chromatography (10% EtOAc in petroleum ether) to afford 3-(3,5-difluorophenyl)acrylaldehyde (230.0 g, 1367.9 mmol, 68% yield) as a pale yellow solid. ¹H NMR (300 MHz, CDCl₃) δ 9.74 (d, J=7.5 Hz, 1H), 7.40 (d, J=15.9 Hz, 1H), 7.13-7.06 (m, 2H), 6.94-6.88 (m, 1H), 6.73 (dd, J=7.2 Hz, 16.2 Hz, 1H).

Step 2

To a suspension of (S)-2-(diphenyl((trimethylsilyl)oxy)methyl)pyrrolidine (11.7 g, 35.8 mmol) in CHCl₃ (600 mL) was added 3-(3,5-difluorophenyl)acrylaldehyde (20.0 g, 119.1 mmol) and tert-butyl hydroxycarbamate (31.7 g, 238.2 mmol). The reaction mixture was stirred at 0° C. for 3 hours. Then MeOH (60 mL) was added to the mixture at 0° C., followed by NaBH₄ (9.1 g, 238.2 mmol) and the mixture was stirred at rt for 16 hours. Concentrated under reduced pressure, the residue was purified by silica gel column chromatography with PE/EA=7/1 to 2/1 to get the crude product as a yellow oil. ¹H NMR (300 MHz, CDCl₃) δ 7.47-7.36 (m, 1H), 6.98-6.91 (m, 2H), 6.75-6.69 (m, 1H), 5.17 (dd, J=5.1, 10.5 Hz, 1H), 3.86-3.73 (m, 2H), 2.76-2.67 (m, 1H), 2.40-2.30 (m, 1H), 2.07-1.98 (m, 1H), 1.44 (s, 9H); MS (m/z) 204.2 (M+H-Boc)⁺, retention time: 1.50 min, UPLC/MS Method 1 using 5% ACN. Chiral HPLC retention time: 7.446 min; Chiral HPLC Analytical Method 14, eluent: 90% (v) hexane+10% EtOH+0.2% (v) DEA.

Step 3

To a solution of (S)-tert-butyl (1-(3,5-difluorophenyl)-3-hydroxypropyl)(hydroxy)carbamate (3.7 g, 12.21 mmol) and triphenylphosphine (6.4 g, 24.42 mmol) in DCM (850 mL) was added a solution of DIAD (4.94 g, 24.42 mmol) in DCM (50 mL) dropwise at 0° C. under N₂. The mixture was stirred at rt for 16 hours. Concentrated, the residue was purified by silica column chromatography using 0-20% EtOAc in petroleum ether to afford (S)-tert-butyl 3-(3,5-difluorophenyl)isoxazolidine-2-carboxylate (2.0 g, 7.0 mmol, yield: 57%) as light yellow oil. ¹H NMR (300 MHz, CDCl₃) δ 6.92-6.85 (m, 2H), 6.72-6.64 (m, 1H), 5.17 (dd, J=7.2, 11.6 Hz, 1H), 4.19-4.12 (m, 2H), 3.90-3.82 (m, 1H), 2.83-2.73 (m, 1H), 2.30-2.18 (m, 1H), 1.47 (s, 9H); MS (m/z) 229.9 (M+H−56)⁺, retention time: 2.27 min, LC/MS Method 23 using 20% ACN.

Step 4

TFA (5 mL) was added slowly to a solution of (S)-tert-butyl 3-(3,5-difluorophenyl)isoxazolidine-2-carboxylate (2 g, 7.02 mmol) in DCM (20 mL) at 0° C. The mixture was stirred at rt for 2 hours. The mixture was concentrated to afford the crude (S)-3-(3,5-difluorophenyl)isoxazolidine as TFA salt (3 g, purity: 70%), which was used for the next step without other further purification. ¹H NMR (300 MHz, CDCl₃) δ 6.97-6.83 (m, 3H), 4.96-4.91 (m, 1H), 4.54-4.46 (m, 1H), 4.38-4.29 (m, 1H), 3.00-2.873 (m, 1H), 2.67-2.56 (m, 1H); MS (m/z) 186.3 (M+H)⁺, retention time: 1.35 min, UPLC/MS Method 1 using 5% ACN.

Step 5

A mixture of crude (S)-3-(3,5-difluorophenyl)isoxazolidine TFA salt (1.25 g, 4.2 mmol, purity: 58%), 1-(6-(methoxycarbonyl)pyrimidin-4-yl)piperidine-4-carboxylic acid (1.34 g, 5.04 mmol), HATU (2.40 g, 6.3 mmol) and DIEA (2.71 g, 21.0 mmol) in DCM (21 mL) was stirred at rt for 16 hours. After completion, 50 mL water was added. The organic phase was washed with saturated aqueous NH₄Cl solution (60 mL×3), dried over anhydrous Na₂SO₄. The filtrate was concentrated, the residue was purified by C18 (5%-95% ACN in H₂O) to afford the crude product (S)-methyl 6-(4-(3-(3,5-difluorophenyl)isoxazolidine-2-carbonyl)piperidin-1-yl)pyrimidine-4-carboxylate (1.5 g, crude) as yellow solid.

¹H NMR (300 MHz, CDCl₃) δ 8.67 (s, 1H), 7.30-7.26 (m, 1H), 6.82-6.80 (m, 2H), 6.73-6.67 (m, 1H), 5.37 (dd, J=6.0 Hz, 8.7 Hz, 1H), 4.48-4.46 (m, 2H), 4.31-4.25 (m, 1H), 3.98 (s, 3H), 3.96-3.88 (m, 1H), 3.22-3.09 (m, 3H), 2.87-2.85 (m, 1H), 2.3.7-2.30 (m, 1H), 2.11-2.04 (m, 1H), 1.86-1.76 (m, 3H); MS (m/z) 433.2 (M+H)⁺, retention time: 1.54 min, LC/MS Method 24 using 5% ACN.

Step 6

A solution of (S)-methyl 6-(4-(3-(3,5-difluorophenyl) isoxazolidine-2-carbonyl) piperidin-1-yl)pyrimidine-4-carboxylate (700 mg, 1.62 mmol) and methylamine in EtOH (2 N in EtOH, 15 mL) was stirred at rt for 16 hours. The solvent was removed under reduced pressure. The filtrate was concentrated, the residue was purified by C18 (5%-95% ACN in H₂O) to afford the product (S)-6-(4-(3-(3,5-difluorophenyl)isoxazolidine-2-carbonyl)piperidin-1-yl)-N-methylpyrimidine-4-carboxamide (600 mg, 1.39 mmol, 86% yield) as white solid. ¹H NMR (400 MHz, CDCl₃) δ 8.51 (s, 1H), 8.02-7.94 (m, 1H), 7.37 (s, 1H), 6.84-6.78 (m, 2H), 6.73-6.67 (m, 1H), 5.37 (dd, J=6.4 Hz, 8.8 Hz, 1H), 4.59-4.43 (m, 2H), 4.31-4.26 (m, 1H), 3.95-3.86 (m, 1H), 3.19-3.07 (m, 3H), 3.00 (d, J=5.2 Hz, 3H), 2.90-2.82 (m, 1H), 2.3.7-2.29 (m, 1H), 2.08-2.04 (m, 1H), 1.86-1.62 (m, 3H); MS (m/z) 432.2 (M+H)⁺, retention time: 3.58 min, UPLC/MS Method 3 using 5% ACN.

### Example 10

### (S)-6-(4-(3-(3,5-difluorophenyl)isoxazolidine-2-carbonyl)piperidin-1-yl)-N,N-dimethylpyrimidine-4-carboxamide

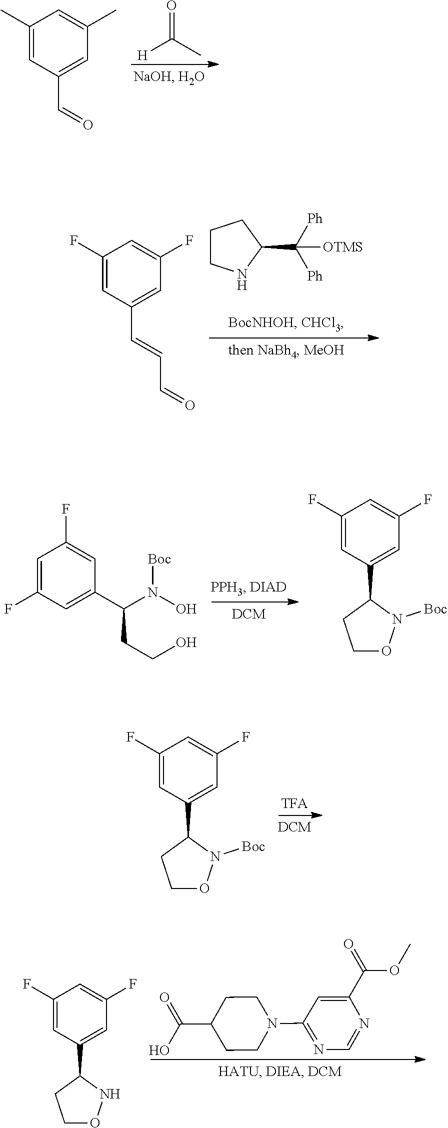

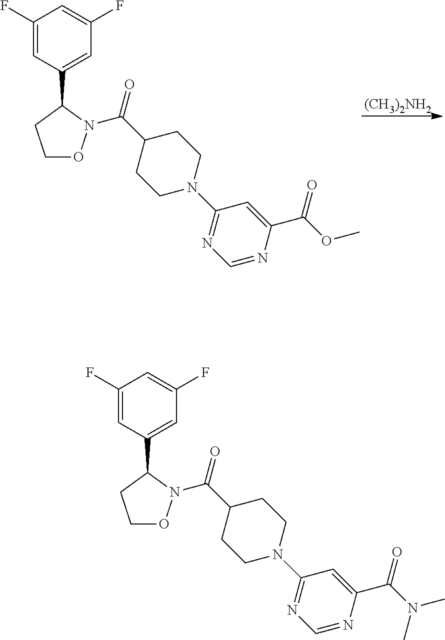 [SMILES: COC(=O)c1cc(N2CCC(C(=O)N3OCC[C@H]3c3cc(F)cc(F)c3)CC2)ncn1 ; "(CH3)2NH2" ; CN(C)C(=O)c1cc(N2CCC(C(=O)N3OCC[C@H]3c3cc(F)cc(F)c3)CC2)ncn1]

Step 1

To a suspension of 3,5-difluorobenzaldehyde (286 g, 2.01 mol) in water (2.5 L) was added acetaldehyde (35% in water, 380 g, 3.01 mol) at 0° C. Then an aqueous solution of sodium hydroxide (1.0 M, 2.1 L, 2.11 mol) was added drop wise over 30 minutes. The mixture was stirred at rt for 48 hours. The resulting mixture was diluted with water (1.0 L), and the aqueous layer was extracted with EtOAc (2 L×3). Combined organic phases were washed with brine (2 L), dried over anhydrous sodium sulfate, and concentrated in vacuo. The resulting residue was purified by normal phase column chromatography (10% EtOAc in petroleum ether) to afford 3-(3,5-difluorophenyl)acrylaldehyde (230.0 g, 1367.9 mmol, 68% yield) as a pale yellow solid. ¹H NMR (300 MHz, CDCl₃) δ 9.74 (d, J=7.5 Hz, 1H), 7.40 (d, J=15.9 Hz, 1H), 7.13-7.06 (m, 2H), 6.94-6.88 (m, 1H), 6.73 (dd, J=7.2 Hz, 16.2 Hz, 1H).

Step 2

To a suspension of (S)-2-(diphenyl((trimethylsilyl)oxy)methyl)pyrrolidine (11.7 g, 35.8 mmol) in CHCl₃ (600 mL) was added 3-(3,5-difluorophenyl)acrylaldehyde (20.0 g, 119.1 mmol) and tert-butyl hydroxycarbamate (31.7 g, 238.2 mmol). The reaction mixture was stirred at 0° C. for 3 hours. Then MeOH (60 mL) was added to the mixture at 0° C., followed by NaBH₄ (9.1 g, 238.2 mmol) and the mixture was stirred at rt for 16 hours. Concentrated under reduced pressure, the residue was purified by silica gel column chromatography with PE/EA=7/1 to 2/1 to get the crude product as a yellow oil. ¹H NMR (300 MHz, CDCl₃) δ 7.47-7.36 (m, 1H), 6.98-6.91 (m, 2H), 6.75-6.69 (m, 1H), 5.17 (dd, J=5.1, 10.5 Hz, 1H), 3.86-3.73 (m, 2H), 2.76-2.67 (m, 1H), 2.40-2.30 (m, 1H), 2.07-1.98 (m, 1H), 1.44 (s, 9H); MS (m/z) 204.2 (M+H-Boc)⁺, retention time: 1.50 min, UPLC/MS Method 1 using 5% ACN. Chiral HPLC retention time: 7.446 min; Chiral HPLC Analytical Method 14, eluent: 90% (v) hexane+10% EtOH+0.2% (v) DEA.

Step 3

To a solution of (S)-tert-butyl (1-(3,5-difluorophenyl)-3-hydroxypropyl)(hydroxy)carbamate (3.7 g, 12.21 mmol) and triphenylphosphine (6.4 g, 24.42 mmol) in DCM (850 mL) was added a solution of DIAD (4.94 g, 24.42 mmol) in DCM (50 mL) dropwise at 0° C. under N₂. The mixture was stirred at rt for 16 hours. Concentrated, the residue was purified by silica column chromatography using 0-20% EtOAc in petroleum ether to afford (S)-tert-butyl 3-(3,5-difluorophenyl)isoxazolidine-2-carboxylate (2.0 g, 7.0 mmol, yield: 57%) as light yellow oil. ¹H NMR (300 MHz, CDCl₃) δ 6.92-6.85 (m, 2H), 6.72-6.64 (m, 1H), 5.17 (dd, J=7.2, 11.6 Hz, 1H), 4.19-4.12 (m, 2H), 3.90-3.82 (m, 1H), 2.83-2.73 (m, 1H), 2.30-2.18 (m, 1H), 1.47 (s, 9H); MS (m/z) 229.9 (M+H−56)⁺, retention time: 2.27 min, LC/MS Method 23 using 20% ACN.

Step 4

TFA (5 mL) was added slowly to a solution of (S)-tert-butyl 3-(3,5-difluorophenyl)isoxazolidine-2-carboxylate (2 g, 7.02 mmol) in DCM (20 mL) at 0° C. The mixture was stirred at rt for 2 hours. The mixture was concentrated to afford the crude (S)-3-(3,5-difluorophenyl)isoxazolidine as TFA salt (3 g, purity: 70%), which was used for the next step without other further purification. ¹H NMR (300 MHz, CDCl₃) δ 6.97-6.83 (m, 3H), 4.96-4.91 (m, 1H), 4.54-4.46 (m, 1H), 4.38-4.29 (m, 1H), 3.00-2.873 (m, 1H), 2.67-2.56 (m, 1H); MS (m/z) 186.3 (M+H)⁺, retention time: 1.35 min, UPLC/MS Method 1 using 5% ACN.

Step 5

A mixture of crude (S)-3-(3,5-difluorophenyl)isoxazolidine TFA salt (1.25 g, 4.2 mmol, purity: 58%), 1-(6-(methoxycarbonyl)pyrimidin-4-yl)piperidine-4-carboxylic acid (1.34 g, 5.04 mmol), HATU (2.40 g, 6.3 mmol) and DIEA (2.71 g, 21.0 mmol) in DCM (21 mL) was stirred at rt for 16 hours. After completion, 50 mL water was added. The organic phase was washed with saturated aqueous NH₄Cl solution (60 mL×3), dried over anhydrous Na₂SO₄. The filtrate was concentrated, the residue was purified by C18 (5%-95% ACN in H₂O) to afford the crude product (S)-methyl 6-(4-(3-(3,5-difluorophenyl)isoxazolidine-2-carbonyl)piperidin-1-yl)pyrimidine-4-carboxylate (1.5 g, crude) as yellow solid.

¹H NMR (300 MHz, CDCl₃) δ 8.67 (s, 1H), 7.30-7.26 (m, 1H), 6.82-6.80 (m, 2H), 6.73-6.67 (m, 1H), 5.37 (dd, J=6.0 Hz, 8.7 Hz, 1H), 4.48-4.46 (m, 2H), 4.31-4.25 (m, 1H), 3.98 (s, 3H), 3.96-3.88 (m, 1H), 3.22-3.09 (m, 3H), 2.87-2.85 (m, 1H), 2.3.7-2.30 (m, 1H), 2.11-2.04 (m, 1H), 1.86-1.76 (m, 3H); MS (m/z) 433.2 (M+H)⁺, retention time: 1.54 min, LC/MS Method 24 using 5% ACN.

Step 6

A solution of (S)-methyl 6-(4-(3-(3,5-difluorophenyl) isoxazolidine-2-carbonyl) piperidin-1-yl)pyrimidine-4-carboxylate (700 mg, 1.62 mmol) and dimethylamine in EtOH (2 N in EtOH, 15 mL) was stirred at rt for 16 hours. The solvent was removed under reduced pressure. The filtrate was concentrated, the residue was purified by C18 (5%-95% ACN in H₂O) to afford the product (S)-6-(4-(3-(3,5-difluorophenyl)isoxazolidine-2-carbonyl)piperidin-1-yl)-N,N-dimethylpyrimidine-4-carboxamide (533 mg, 1.20 mmol, 65% yield) as white solid. ¹H NMR (400 MHz, CDCl₃) δ 8.56 (s, 1H), 6.84-6.79 (m, 3H), 6.73-6.67 (m, 1H), 5.36 (dd, J=6.4 Hz, 9.2 Hz, 1H), 4.52-4.34 (m, 2H), 4.31-4.26 (m, 1H), 3.95-3.88 (m, 1H), 3.17-3.04 (m, 9H), 2.90-2.82 (m, 1H), 2.37-2.29 (m, 1H), 2.08-2.04 (m, 1H), 1.86-1.74 (m, 3H); MS (m/z) 446.0 (M+H)⁺, retention time: 3.14 min, UPLC/MS Method 3 using 5% ACN.

### Example 11 and Example 12

### 1-((3R,4S)-4-((S)-3-(3,5-difluorophenyl)isoxazolidine-2-carbonyl)-3-fluoropiperidin-1-yl)ethan-1-one

### and

### 1-((3S,4R)-4-((R)-3-(3,5-difluorophenyl)isoxazolidine-2-carbonyl)-3-fluoropiperidin-1-yl)ethan-1-one

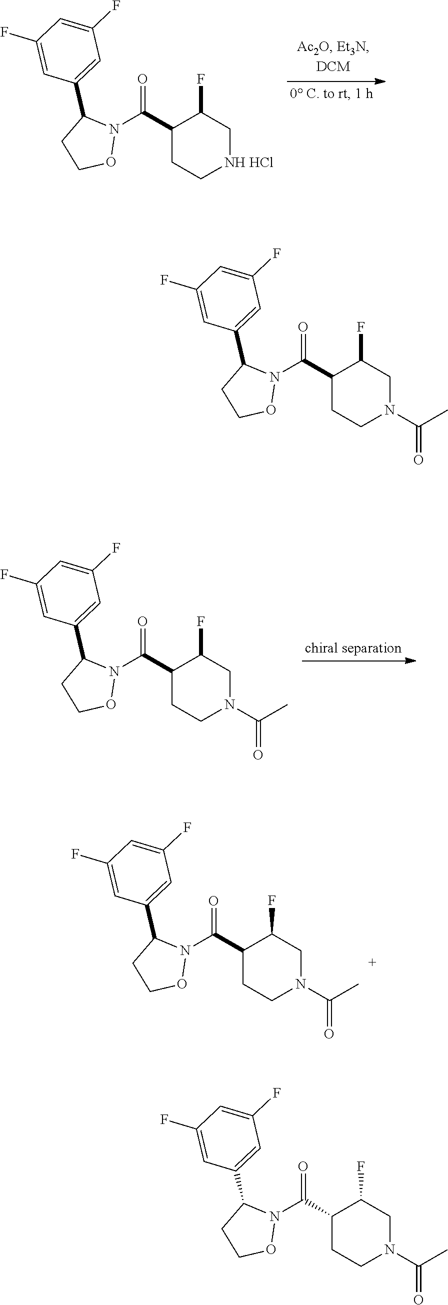

Step 1:

Ac₂O (80 mg, 0.78 mmol) was added into a stirring mixture of cis-(3-(3,5-difluorophenyl)isoxazolidin-2-yl)(3-fluoropiperidin-4-yl)methanone hydrochloride (250 mg, 0.71 mmol) and Et₃N (215 mg, 2.13 mmol) in DCM (7 mL) at 0° C. The resulting mixture was stirred at rt for 1 hour, quenched by addition of H₂O (30 mL), and extracted with EA (3×20 mL). The combined organic layers were washed with brine (100 mL), dried over anhydrous Na₂SO₄, filtered, and concentrated under vacuum. The residue was purified by Prep-HPLC with the condition: Column: XBridge Prep OBD C18 Column 30×150 mm, 5 um; Mobile Phase A: Water (10 mmol/L NH₄HCO₃), Mobile Phase B: ACN; Flow rate: 60 mL/min; Gradient: 15% B to 55% B in 7 min; 254 nm; Rt: 5.83 min to afford cis 1-(4-(3-(3,5-difluorophenyl)isoxazolidine-2-carbonyl)-3-fluoropiperidin-1-yl)ethan-1-one (200 mg, 78% yield) as an off-white solid. LCMS (m/z) 357 (M+H)⁺, retention time: 0.721 min, LC/MS Method 14.

Step 2:

The racemate of cis-1-((3R,4S)-4-((S)-3-(3,5-difluorophenyl)isoxazolidine-2-carbonyl)-3-fluoropiperidin-1-yl)ethan-1-one (200 mg, 0.56 mmol) were separated by Prep-Chiral-HPLC with the following conditions: Column: CHIRAL ART Cellulose-SB S-5 um 50×250 mm, 50 mm×250 mm, 5 um; Mobile Phase A: Hexane: DCM=5:1, Mobile Phase B: IPA; Flow rate: 20 mL/min; Gradient: 30 B to 30 B in 15 min; UV254 & 220 nm; Rt1: 8.943 min; Rt2: 12.188 min to afford two title compounds:

Example 11: 1-((3R,4S)-4-((S)-3-(3,5-difluorophenyl)isoxazolidine-2-carbonyl)-3-fluoropiperidin-1-yl)ethan-1-one was obtained as a off white solid (43.4 mg, 43% yield). LCMS (m/z) 357 (M+H)⁺, retention time: 2.170 min, LC/MS Method 17. Chiral HPLC Method 4, retention time: 3.154 min, % ee=100%. ¹H NMR (300 MHz, DMSO-d₆) ppm 7.10-6.87 (m, 3H), 5.38 (dd, J=9.0, 6.0 Hz, 1H), 5.03 (d, J=48 Hz, 1H), 4.65-3.72 (m, 4H), 3.53-3.10 (m, 2H), 2.97-2.60 (m, 2H), 2.35-2.22 (m, 1H), 2.05-1.79 (m, 4H), 1.75-1.59 (m, 1H).

Example 12: 1-((3S,4R)-4-((R)-3-(3,5-difluorophenyl)isoxazolidine-2-carbonyl)-3-fluoropiperidin-1-yl)ethan-1-one was obtained as a off white solid (43.3 mg, 43% yield). LCMS (m/z) 357 (M+H)⁺, retention time: 1.249 min, LC/MS Method 18. Chiral HPLC Method 4, retention time: 4.722 min, % ee=100%. ¹H NMR (300 MHz, DMSO-d₆) ppm 7.10-6.95 (m, 3H), 5.48-5.28 (m, 1H), 5.02 (d, J=48 Hz, 1H), 4.62-3.73 (m, 4H), 3.73-3.07 (m, 2H), 2.98-2.60 (m, 2H), 2.35-2.13 (m, 1H), 2.09-1.80 (m, 4H), 1.80-1.60 (m, 1H).

### Example 13 and Example 14

### 1-((3S,4R)-4-((S)-3-(3,5-difluorophenyl)isoxazolidine-2-carbonyl)-3-fluoropiperidin-1-yl)ethan-1-one

### and

### 1-((3R,4S)-4-((R)-3-(3,5-difluorophenyl)isoxazolidine-2-carbonyl)-3-fluoropiperidin-1-yl)ethan-1-one

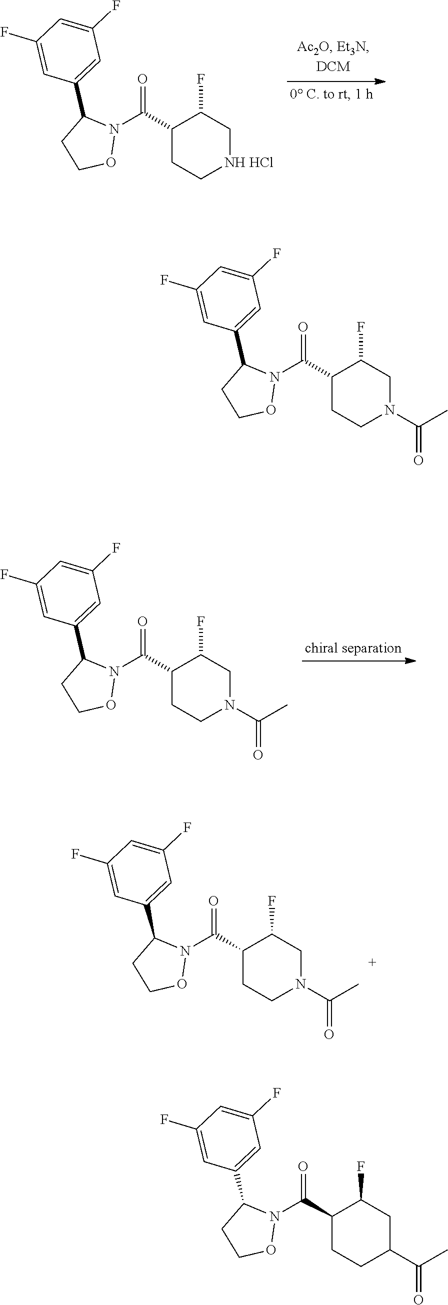

Step 1:

Ac₂O (64 mg, 0.62 mmol) was added into a stirring mixture of trans-(3-(3,5-difluorophenyl)isoxazolidin-2-yl)(3-fluoropiperidin-4-yl)methanone hydrochloride (200 mg, 0.57 mmol) and Et₃N (173 mg, 1.71 mmol) in DCM (5 mL) at 0° C. The resulting mixture was stirred at rt for 1 hour, quenched by addition of H₂O (30 mL), and extracted with EA (3×20 mL). The combined organic layers were washed with brine (50 mL), dried over anhydrous Na₂SO₄, filtered, and concentrated under vacuum. The residue was purified by Prep-HPLC with the column: Column: XBridge Prep OBD C18 Column 30×150 mm, 5 um; Mobile Phase A: Water (10 mmol/L NH₄HCO₃), Mobile Phase B: ACN; Flow rate: 60 mL/min; Gradient: 15% B to 58% B in 7 min; 254 nm; Rt: 5.88 min to afford trans 1-(4-(3-(3,5-difluorophenyl)isoxazolidine-2-carbonyl)-3-fluoropiperidin-1-yl)ethan-1-one (150 mg, 73% yield) as off white solid. LCMS (m/z) 357 (M+H)⁺, retention time: 0.726 min, LC/MS Method 14.

Step 2:

The racemate of trans-1-(4-(3-(3,5-difluorophenyl)isoxazolidine-2-carbonyl)-3-fluoropiperidin-1-yl)ethan-1-one (150 mg, 0.42 mmol) were separated by Prep-Chiral-HPLC with the following conditions: Column: CHIRALPAK IF, 2×25 cm, 5 um; Mobile Phase A: Hexane: DCM=5:1, Mobile Phase B: EtOH; Flow rate: 20 mL/min; Gradient: 30 B to 30 B in 15 min; 254/220 nm; Rt1: 8.995 min; Rt2: 10.763 min to afford two title compounds:

Example 13: 1-((3S,4R)-4-((S)-3-(3,5-difluorophenyl)isoxazolidine-2-carbonyl)-3-fluoropiperidin-1-yl)ethan-1-one: (43.6 mg, 57% yield) as off white solid. LCMS (m/z) 357 (M+H)⁺, retention time: 1.255 min, LC/MS Method 18. Chiral HPLC Method 4, retention time: 1.723 min, % ee=100%. ¹H NMR (300 MHz, DMSO-d₆) ppm 7.10-6.85 (m, 3H), 5.38 (dd, J=9.0, 6.0 Hz, 1H), 5.20 (d, J=48 Hz, 1H), 4.81-3.63 (m, 4H), 3.63-3.08 (m, 2H), 2.95-2.55 (m, 2H), 2.32-2.12 (m, 1H), 2.09-1.70 (m, 1H), 1.70-1.46 (m, 1H).

Example 14: 1-((3R,4S)-4-((R)-3-(3,5-difluorophenyl)isoxazolidine-2-carbonyl)-3-fluoropiperidin-1-yl)ethan-1-one: (49.1 mg, 65% yield) as off white solid. LCMS (m/z) 357 (M+H)⁺, retention time: 1.253 min, LC/MS Method 18. Chiral HPLC Method 4, retention time: 2.153 min, % ee=100%. ¹H NMR (300 MHz, DMSO-d₆) ppm 7.11-6.82 (m, 3H), 5.49-5.35 (m, 1H), 5.2 (d, J=48 Hz, 1H), 4.91-3.68 (m, 4H), 3.68-3.08 (m, 2H), 2.95-2.60 (m, 2H), 2.35-2.12 (m, 1H), 2.05-1.70 (m, 1H), 1.70-1.47 (m, 1H).

The following compounds (Examples 15-48) were synthesized from cis-(3-(3,5-difluorophenyl)isoxazolidin-2-yl)(3-fluoropiperidin-4-yl)methanone hydrochloride and trans-(3-(3,5-difluorophenyl)isoxazolidin-2-yl)(3-fluoropiperidin-4-yl)methanone hydrochloride (Intermediate 3) in an analogous manner to the synthesis of Examples 3-8. For all the examples, displacement took place first, followed by chiral separation.

| | | | | Chiral | | |
|---|---|---|---|---|---|---|
| | | | | HPLC/SFC: | LC: | |
| | | | | retention | retention | |
| | | | | time (min; | Time | |
| | | | | Chiral | (min); | |
| | | | | HPLC/SFC | LC/MS | |
| | | | | Analytic | Method of | MS |
| Ex. | Name | Structure | ¹H NMR | Method | analysis | (M + H)⁺ |
| 15 | ((S)-3-(3,5- difluorophenyl) isoxazolidin-2- yl)((3R,4S)-3- fluoro-1-(5- methyl-1,3,4- oxadiazol-2- yl)piperidin-4- yl)methanone | 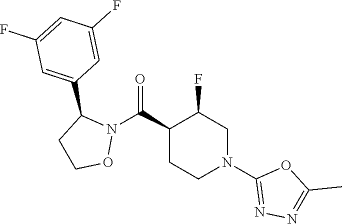 [SMILES: Cc1nnc(N2CC[C@@H](C(=O)N3OCC[C@H]3c3cc(F)cc(F)c3)[C@@H](F)C2)o1] | ¹H NMR (300 MHz, CDCl₃-d) ppm 6.86-6.83 (m, 2H), 6.76-6.70 (m, 1H), 5.44-5.39 (m, 1H), 5.19-5.04 (m, 1H), 4.35-4.27 (m, 2H), 4.12-3.97 (m, 2H), 3.49-3.20 (m, 3H), 2.93-2.90 (m, 1H), 2.42-2.31 (m, 5H), 1.92-1.86 (m, 1H). | 3.081 min, Chiral HPLC Method 9 | 1.203 min, LC/MS Method 16 | 397 |
| 16 | ((R)-3-(3,5- difluorophenyl) isoxazolidin-2- yl)((3S,4R)-3- fluoro-1-(5- methyl-1,3,4- oxadiazol-2- yl)piperidin-4- yl)methanone | 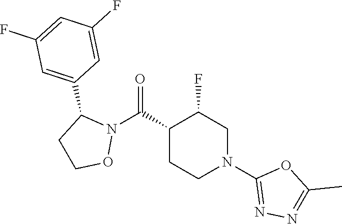 [SMILES: Cc1nnc(N2CC[C@H](C(=O)N3OCC[C@@H]3c3cc(F)cc(F)c3)[C@H](F)C2)o1] | ¹H NMR (300 MHz, CDCl₃-d) ppm 6.86-6.83 (m, 2H), 6.76-6.70 (m, 1H), 5.44-5.39 (m, 1H), 5.19-5.03 (m, 1H), 4.35-4.27 (m, 2H), 4.12-3.97 (m, 2H), 3.49-3.20 (m, 3H), 2.93-2.90 (m, 1H), 2.41-2.31 (m, 5H), 1.92-1.86 (m, 1H). | 3.870 min, Chiral HPLC Method 9 | 1.202 min, LC/MS Method 16 | 397 |
| 17 | ((S)-3-(3,5- difluorophenyl) isoxazolidin-2- yl)((3S,4R)-3- fluoro-1-(5- methyl-1,3,4- oxadiazol-2- yl)piperidin-4- yl)methanone | 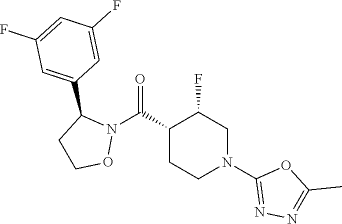 [SMILES: Cc1nnc(N2CC[C@H](C(=O)N3OCC[C@H]3c3cc(F)cc(F)c3)[C@H](F)C2)o1] | ¹H NMR (300 MHz, CDCl₃-d) ppm 6.84-6.80 (m, 2H), 6.73-6.68 (m, 1H), 5.46-5.23 (m, 2H), 4.31-4.14 (m, 3H), 3.97-3.89 (m, 1H), 3.40-2.86 (m, 4H), 2.40-2.33 (m 5H), 1.78-1.72 (m, 1H). | 1.847 min, Chiral HPLC Method 10 | 1.216 min, LC/MS Method 16 | 397 |
| 18 | ((R)-3-(3,5- difluorophenyl) isoxazolidin-2- yl)((3R,4S)-3- fluoro-1-(5- methyl-1,3,4- oxadiazol-2- yl)piperidin-4- yl)methanone | 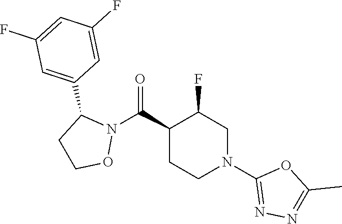 [SMILES: Cc1nnc(N2CC[C@@H](C(=O)N3OCC[C@@H]3c3cc(F)cc(F)c3)[C@@H](F)C2)o1] | ¹H NMR (300 MHz, CDCl₃-d) ppm 6.84-6.81 (m, 2H), 6.75-6.69 (m, 1H), 5.47-5.24 (m, 2H), 4.32-4.15 (m, 3H), 3.98-3.90 (m, 1H), 3.40-2.87 (m, 4H), 2.42-2.32 (m, 5H), 1.79-1.73 (m, 1H). | 2.549 min, Chiral HPLC Method 10 | 1.211 min, LC/MS Method 16 | 397 |
| 19 | ((S)-3-(3,5- difluorophenyl) isoxazolidin-2- yl)((3S,4R)-3- fluoro-1-(5- methyl-1,3,4- thiadiazol-2- yl)piperidin-4- yl)methanone | 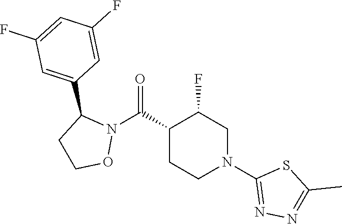 [SMILES: Cc1nnc(N2CC[C@H](C(=O)N3OCC[C@H]3c3cc(F)cc(F)c3)[C@H](F)C2)s1] | ¹H NMR (300 MHz, CDCl₃-d) ppm 6.84-6.82 (m, 2H), 6.76-6.69 (m, 1H), 5.47-5.27 (m, 2H), 4.33-3.91 (m, 4H), 3.50-2.87 (m, 4H), 2.60 (s, 3H), 2.45-2.33 (m, 2H), 1.81-1.78 (m, 1H). | 2.353 min, Chiral HPLC Method 10 | 2.537 min, LC/MS Method 16 | 413 |
| 20 | ((R)-3-(3,5- difluorophenyl) isoxazolidin-2- yl)((3R,4S)-3- fluoro-1-(5- methyl-1,3,4- thiadiazol-2- yl)piperidin-4- yl)methanone | 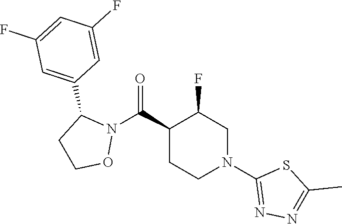 [SMILES: Cc1nnc(N2CC[C@@H](C(=O)N3OCC[C@@H]3c3cc(F)cc(F)c3)[C@@H](F)C2)s1] | ¹H NMR (300 MHz, CDCl₃-d) ppm 6.84-6.82 (m, 2H), 6.76-6.69 (m, 1H), 5.47-5.27 (m, 2H), 4.33-3.91 (m, 4H), 3.51-2.87 (m, 4H), 2.60 (s, 3H), 2.45-2.33 (m, 2H), 1.82-1.78 (m, 1H). | 2.928 min, Chiral HPLC Method 10 | 1.252 min, LC/MS Method 16 | 413 |
| 21 | ((S)-3-(3,5- difluorophenyl) isoxazolidin-2- yl)((3R,4S)-3- fluoro-1-(1,3,4- oxadiazol-2- yl)piperidin-4- yl)methanone | 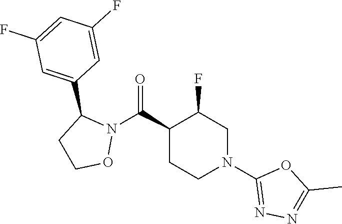 | ¹H NMR (300 MHz, CDCl₃-d) ppm 7.92 (s, 1H), 6.84-6.74 (m, 2H), 6.72-6.68 (m, 1H), 5.42-5.37 (m, 1H), 5.18-5.02 (d, J = 47.7 Hz, 1H), 4.34-4.26 (m, 2H), 4.16-4.11 (m, 1H), 4.03-3.95 (m, 1H), 3.52-3.24 (m, 3H), 2.90-2.87 (m, 1H), 2.41-2.29 (m, 2H), 1.92-1.85 (m, 1H) | 2.833 min, Chiral HPLC Method 6 | 2.256 min, LC/MS Method 12 | 383 |
| 22 | ((R)-3-(3,5- difluorophenyl) isoxazolidin-2- yl)((3S,4R)-3- fluoro-1-(1,3,4- oxadiazol-2- yl)piperidin-4- yl)methanone | 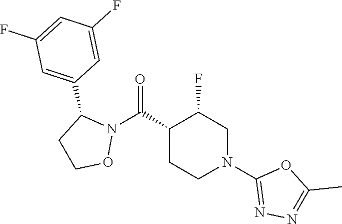 | ¹H NMR (300 MHz, CDCl₃-d) ppm 7.92 (s, 1H), 6.83-6.74 (m, 2H), 6.73-6.68 (m, 1H), 5.42-5.37 (m, 1H, 5.19-5.03 (d, J = 47.7 Hz, 1H), 4.38-4.26 (m, 2H), 4.18-4.11 (m, 1H), 4.03-3.95 (m, 1H), 3.53-3.20 (m, 3H), 2.91-2.84 (m, 1H, 2.41-2.30 (m, 2H), 1.92-1.86 (m, 1H) | 3.692 min, Chiral HPLC Method 6 | 2.267 min, LC/MS Method 12 | 383 |
| 23 | ((S)-3-(3,5- difluorophenyl) isoxazolidin-2- yl)((3S,4R)-3- fluoro-1-(1,3,4- oxadiazol-2- yl)piperidin-4- yl)methanone | 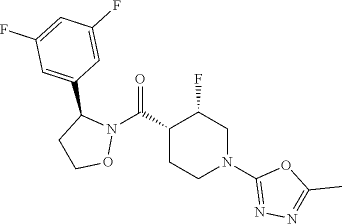 | ¹H NMR (300 MHz, CDCl₃-d) ppm 7.92 (s, 1H), 6.82-6.74 (m, 2H), 6.73-6.67 (m, 1H), 5.45-5.24 (m, 2H), 4.37-4.22 (m, 3H), 3.97-3.89 (m, 1H), 3.48-3.12 (m, 3H), 2.90-2.84 (m, 1H), 2.41-2.32 (m, 2H), 1.80-1.74 (m, 1H) | 3.920 min, Chiral HPLC Method 6 | 2.300 min, LC/MS Method 12 | 383 |
| 24 | ((R)-3-(3,5- difluorophenyl) isoxazolidin-2- yl)((3R,4S)-3- fluoro-1-(1,3,4- oxadiazol-2- yl)piperidin-4- yl)methanone | 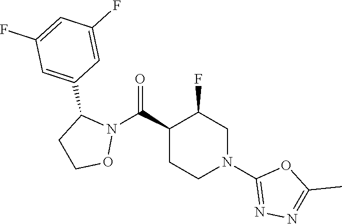 | ¹H NMR (300 MHz, CDCl₃-d) ppm 7.92 (s, 1H), 6.82-6.74 (m, 2H), 6.73-6.67 (m, 1H), 5.45-5.23 (m, 2H), 4.41-4.21 (m, 3H), 3.97-3.89 (m, 1H), 3.44-3.11 (m, 3H), 3.02-2.82 (m, 1H), 2.41-2.32 (m, 2H), 1.79-1.74 (m, 1H) | 4.345 min, Chiral HPLC Method 6 | 2.306 min, LC/MS Method 12 | 383 |
| 25 | 6-((3R,4S)-4- ((S)-3-(3,5- difluorophenyl) isoxazolidine-2- carbonyl)-3- fluoropiperidin- 1-yl)pyrimidine- 4-carboxamide | 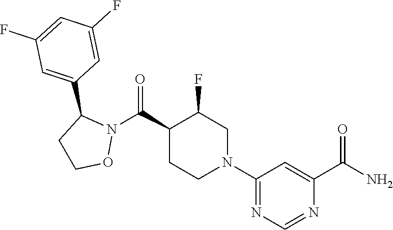 [SMILES: NC(=O)c1cc(N2CC[C@@H](C(=O)N3OCC[C@H]3c3cc(F)cc(F)c3)[C@@H](F)C2)ncn1] | ¹H NMR (300 MHz, CDCl₃) ppm 8.60 (s, 1H), 7.92 (s, 1H), 7.46 (s, 1H), 6.86-6.84 (m, 2H), 6.76-6.67 (m, 1H), 5.67 (s, 1H), 5.45-5.40 (m, 1H, 5.14 (d, J = 47.4 Hz, 1H), 4.80 (s, 1H), 4.50 (s, 1H), 4.35-4.29 (m, 1H), 4.06-3.98 (m, 1H), 3.57-3.31 (m, 3H), 2.93-2.90 (m, 1H), 2.41-2.29 (m, 2H), 1.94-1.87 (m, 1H) | 3.056 min, Chiral HPLC Method 4 | 2.427 min, LC/MS Method 21 | 436 |
| 26 | 6-((3S,4R)-4- ((R)-3-(3,5- difluorophenyl) isoxazolidine-2- carbonyl)-3- fluoropiperidin- 1-yl)pyrimidine- 4-carboxamide | 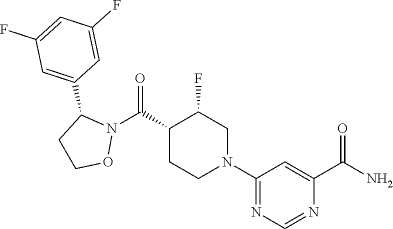 [SMILES: NC(=O)c1cc(N2CC[C@H](C(=O)N3OCC[C@@H]3c3cc(F)cc(F)c3)[C@H](F)C2)ncn1] | ¹H NMR (300 MHz, CDCl₃) ppm 8.60 (s, 1H), 7.92 (s, 1H), 7.47 (s, 1H), 6.87-6.84 (m, 2H), 6.76-6.68 (m, 1H), 5.68 (s, 1H), 5.45-5.40 (m, 1H, 5.15 (d, J = 47.4 Hz, 1H), 4.80 (s, 1H), 4.50 (s, 1H), 4.36-4.29 (m, 1H), 4.06-3.98 (m, 1H), 3.57-3.31 (m, 3H), 2.93-2.90 (m, 1H), 2.41-2.28 (m, 2H), 1.94-1.88 (m, 1H) | 6.294 min, Chiral HPLC Method 4 | 1.317 min, LC/MS Method 8 | 436 |
| 27 | 6-((3S,4R)-4- ((S)-3-(3,5- difluorophenyl) isoxazolidine-2- carbonyl)-3- fluoropiperidin- 1-yl)pyrimidine- 4-carboxamide | 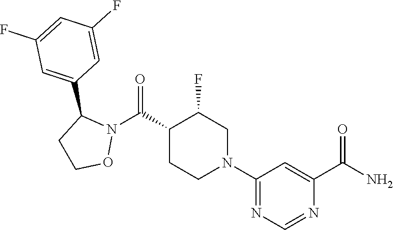 [SMILES: NC(=O)c1cc(N2CC[C@H](C(=O)N3OCC[C@H]3c3cc(F)cc(F)c3)[C@H](F)C2)ncn1] | ¹H NMR (300 MHz, CDCl₃) ppm 8.60 (s, 1H), 7.94 (s, 1H), 7.48 (s, 1H), 6.84-6.82 (m, 2H), 6.75-6.69 (m, 1H), 5.68 (s, 1H), 5.48-5.43 (m, 1H), 5.38 (d, J = 47.4 Hz, 1H), 5.00 (s, 1H), 4.65 (s, 1H), 4.34-4.28 (m, 1H), 4.01-3.93 (m, 1H), 3.39-3.08 (m, 3H), 2.91-2.88 (m, 1H), 2.41-2.29 (m, 2H), 1.85-1.79 (m, 1H) | 2.470 min, Chiral HPLC Method 11 | 1.273 min, LC/MS Method 8 | 436 |
| 28 | 6-((3R,4S)-4- ((R)-3-(3,5- difluorophenyl) isoxazolidine-2- carbonyl)-3- fluoropiperidin- 1-yl)pyrimidine- 4-carboxamide | 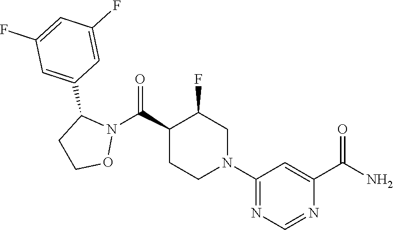 [SMILES: NC(=O)c1cc(N2CC[C@@H](C(=O)N3OCC[C@@H]3c3cc(F)cc(F)c3)[C@@H](F)C2)ncn1] | ¹H NMR (300 MHz, CDCl₃) ppm 8.60 (s, 1H), 7.90 (s, 1H), 7.47 (s, 1H), 6.84-6.82 (m, 2H), 6.75-6.69 (m, 1H), 5.70 (S. 1H), 5.48-5.43 (m, 1H), 5.38 (d, J = 47.4 Hz, 1H), 5.00 (s, 1H), 4.65 (s, 1H), 4.34-4.28 (m, 1H), 4.00-3.93 (m, 1H), 3.39-3.08 (m, 3H), 2.91-2.88 (m, 1H), 2.41-2.29 (m, 2H), 1.85-1.79 (m, 1H) | 3.414 min, Chiral HPLC Method 11 | 1.290 min, LC/MS Method 8 | 436 |
| 29 | 6-((3R,4S)-4- ((S)-3-(3,5- difluorophenyl) isoxazolidine-2- carbonyl)-3- fluoropiperidin- 1-yl)-N-methyl- pyrimidine-4- carboxamide | 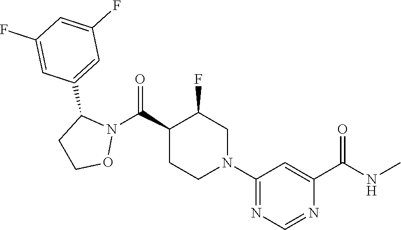 [SMILES: CNC(=O)c1cc(N2CC[C@@H](C(=O)N3OCC[C@@H]3c3cc(F)cc(F)c3)[C@@H](F)C2)ncn1] | ¹H NMR (300 MHz, CDCl₃-d) ppm 8.58 (s, 1H), 8.16 (s, 1H), 7.48 (s, 1H), 6.90-6.82 (m, 2H), 6.80-6.69 (m, 1H), 5.46-5.40 (m, 1H), 5.24-5.07 (m, 1H), 4.91-4.72 (brs, 1H), 4.62-4.43 (brs, 1H), 4.37-4.30 (m, 1H), 4.07-3.99 (m, 1H), 3.59-3.26 (m, 3H), 3.04 (d, J = 5.1 Hz, 3H), 2.99-2.87 (m, 1H), 2.45-2.23 (m, 2H), 1.97-1.87 (m, 1H). | 2.235 min, Chiral HPLC Method 8 | 0.969 min, LC/MS Method 12 | 450 |
| 30 | 6-((3S,4R)-4- ((R)-3-(3,5- difluorophenyl) isoxazolidine-2- carbonyl)-3- fluoropiperidin- 1-yl)-N-methyl- pyrimidine-4- carboxamide | 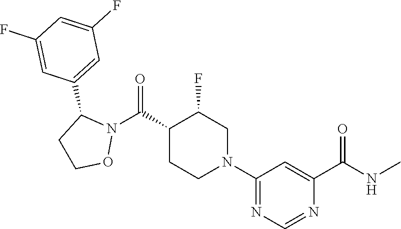 [SMILES: CNC(=O)c1cc(N2CC[C@H](C(=O)N3OCC[C@@H]3c3cc(F)cc(F)c3)[C@H](F)C2)ncn1] | ¹H NMR (300 MHz, CDCl₃-d) ppm 8.57 (s, 1H), 8.10 (s, 1H), 7.47 (s, 1H), 6.90-6.82 (m, 2H), 6.78-6.70 (m, 1H), 5.46-5.40 (m, 1H), 5.23-5.07 (m, 1H), 4.90-4.70 (brs, 1H), 4.61-4.41 (brs, 1H), 4.37-4.30 (m, 1H), 4.07-3.99 (m, 1H), 3.58-3.26 (m, 3H, 3.04 (d, J = 5.1 Hz, 3H), 3.00-2.88 (m, 1H), 2.45-2.23 (m, 2H), 1.97-1.87 (m, 1H). | 5.334 min, Chiral HPLC Method 8 | 0.973 min, LC/MS Method 12 | 450 |
| 31 | 6-((3S,4R)-4- ((S)-3-(3,5- difluorophenyl) isoxazolidine-2- carbonyl)-3- fluoropiperidin- 1-yl)-N-methyl- pyrimidine-4- carboxamide | 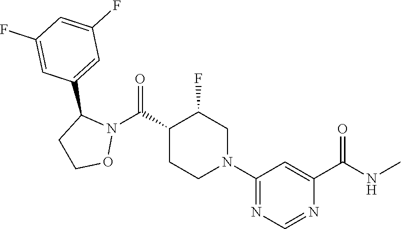 [SMILES: CNC(=O)c1cc(N2CC[C@H](C(=O)N3OCC[C@H]3c3cc(F)cc(F)c3)[C@H](F)C2)ncn1] | ¹H NMR (300 MHz, CDCl₃-d) ppm 8.57 (S, 1H), 8.07 (s, 1H), 7.47 (s, 1H), 6.87-6.79 (m, 2H), 6.76-6.67 (m, 1H), 5.48-5.27 (m, 2H), 5.13-4.89 (brs, 1H), 4.75-4.56 (brs, 1H), 4.34-4.27 (m, 1H), 4.01-3.92 (m, 1H), 3.39-3.06 (m, 3H), 3.03 (d, J = 5.1 Hz, 3H), 2.95-2.84 (m, 1H), 2.44-2.22 (m, 2H), 1.86-1.78 (m, 1H). | 1.516 min, Chiral HPLC Method 7 | 0.970 min, LC/MS Method 12 | 450 |
| 32 | 6-((3R,4S)-4- ((R)-3-(3,5- difluorophenyl) isoxazolidine-2- carbonyl)-3- fluoropiperidin- 1-yl)-N-methyl- pyrimidine-4- carboxamide | 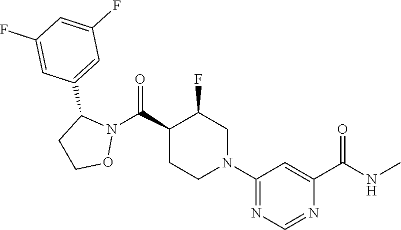 [SMILES: CNC(=O)c1cc(N2CC[C@@H](C(=O)N3OCC[C@@H]3c3cc(F)cc(F)c3)[C@@H](F)C2)ncn1] | ¹H NMR (300 MHz, CDCl₃-d) ppm 8.57 (s, 1H), 8.15 (s, 1H), 7.48 (s, 1H), 6.87-6.79 (m, 2H), 6.76-6.68 (m, 1H), 5.48-5.29 (m, 2H), 5.14-4.89 (brs, 1H), 4.75-4.55 (brs, 1H), 4.35-4.27 (m, 1H), 4.01-3.92 (m, 1H), 3.40-3.08 (m, 3H), 3.03 (d, J = 5.1 Hz, 3H), 2.95-2.84 (m, 1H), 2.44-2.22 (m, 2H), 1.87-1.78 (m, 1H). | 1.782 min, Chiral HPLC Method 7 | 2.167 min, LC/MS Method 19 | 450 |
| 33 | 6-((3R,4S)-4- ((S)-3-(3,5- difluorophenyl) isoxazolidine-2- carbonyl)-3- fluoropiperidin- 1-yl)-N,N- dimethyl- pyrimidine-4- carboxamide | 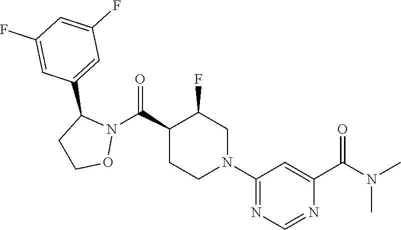 [SMILES: CN(C)C(=O)c1cc(N2CC[C@@H](C(=O)N3OCC[C@H]3c3cc(F)cc(F)c3)[C@@H](F)C2)ncn1] | ¹H NMR (300 MHz, CDCl₃-d) ppm 8.61 (s, 1H), 6.90-6.81 (m, 3H), 6.77-6.69 (m, 1H), 5.45-5.39 (m, 1H), 5.23-5.05 (m, 1H), 4.87-4.66 (brs, 1H), 4.48-4.29 (m, 2H), 4.06-3.98 (m, 1H), 3.54-3.22 (m, 3H, 3.11 (d, J = 5.7 Hz, 6H), 2.98-2.86 (m, 1H), 2.44-2.22 (m, 2H), 1.95-1.85 (m, 1H). | 3.436 min, Chiral HPLC Method 6 | 1.077 min, LC/MS Method 16 | 464 |
| 34 | 6-((3S,4R)-4- ((R)-3-(3,5- difluorophenyl) isoxazolidine-2- carbonyl)-3- fluoropiperidin- 1-yl)-N,N- dimethyl- pyrimidine-4- carboxamide | 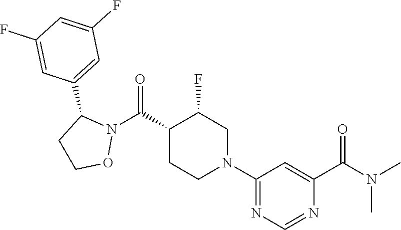 [SMILES: CN(C)C(=O)c1cc(N2CC[C@H](C(=O)N3OCC[C@@H]3c3cc(F)cc(F)c3)[C@H](F)C2)ncn1] | ¹H NMR (300 MHz, CDCl₃-d) ppm 8.61 (s, 1H), 6.89-6.82 (m, 3H), 6.77-6.69 (m, 1H), 5.44-5.39 (m, 1H), 5.23-5.04 (m, 1H), 4.84-4.66 (brs, 1H), 4.46-4.29 (m, 2H), 4.06-3.98 (m, 1H), 3.53-3.21 (m, 3H, 3.11 (d, J = 6.3 Hz, 6H), 2.98-2.86 (m, 1H), 2.44-2.22 (m, 2H), 1.95-1.85 (m, 1H). | 4.575 min, Chiral HPLC Method 6 | 1.074 min, LC/MS Method 16 | 464 |
| 35 | 6-((3S,4R)-4- ((S)-3-(3,5- difluorophenyl) isoxazolidine-2- carbonyl)-3- fluoropiperidin- 1-yl)-N,N- dimethyl- pyrimidine-4- carboxamide | 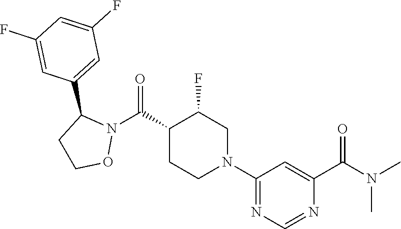 [SMILES: CN(C)C(=O)c1cc(N2CC[C@H](C(=O)N3OCC[C@H]3c3cc(F)cc(F)c3)[C@H](F)C2)ncn1] | ¹H NMR (300 MHz, CDCl₃-d) ppm 8.61 (s, 1H), 6.87-6.79 (m, 3H), 6.76-6.67 (m, 1H), 5.48-5.28 (m, 2H), 5.03-4.79 (brs, 1H), 4.69-4.46 (brs, 1H), 4.34-4.27 (m, 1H), 4.01-3.92 (m, 1H), 3.36-3.03 (m, 9H), 2.95-2.84 (m, 1H), 2.44-2.21 (m, 2H), 1.85-1.76 (m, 1H). | 3.871 min, Chiral HPLC Method 6 | 1.065 min, LC/MS Method 16 | 464 |
| 36 | 6-((3R,4S)-4- ((R)-3-(3,5- difluorophenyl) isoxazolidine-2- carbonyl)-3- fluoropiperidin- 1-yl)-N,N- dimethyl- pyrimidine-4- carboxamide | 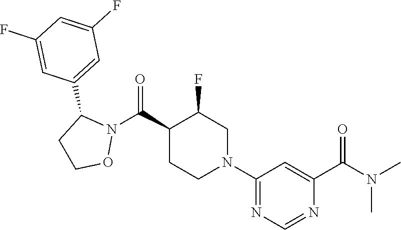 [SMILES: CN(C)C(=O)c1cc(N2CC[C@@H](C(=O)N3OCC[C@@H]3c3cc(F)cc(F)c3)[C@@H](F)C2)ncn1] | ¹H NMR (300 MHz, CDCl₃-d) ppm 8.61 (s, 1H), 6.87-6.79 (m, 3H), 6.76-6.67 (m, 1H), 5.48-5.28 (m, 2H), 5.02-4.80 (brs, 1H), 4.66-4.49 (brs, 1H), 4.35-4.27 (m, 1H), 4.01-3.92 (m, 1H), 3.37-3.03 (m, 9H), 2.96-2.84 (m, 1H), 2.44-2.22 (m, 2H), 1.85-1.77 (m, 1H). | 4.338 min, Chiral HPLC Method 6 | 1.076 min, LC/MS Method 16 | 464 |
| 37 | ((S)-3-(3,5- difluorophenyl) isoxazolidin-2- yl)((3R,4S)-3- fluoro-1-(5- (methylsulfonyl) pyrimidin-2- yl)piperidin-4- yl)methanone | 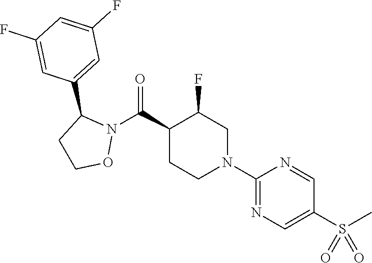 [SMILES: CS(=O)(=O)c1cnc(N2CC[C@@H](C(=O)N3OCC[C@H]3c3cc(F)cc(F)c3)[C@@H](F)C2)nc1] | ¹H NMR (300 MHz, CDCl₃-d) ppm 8.73 (s, 2H), 6.97-6.81 (m, 2H), 6.81-6.49 (m, 1H), 5.42 (dd, J = 9.0, 6.0 Hz, 1H), 5.29-5.02 (m, 2H), 5.02-4.80 (m, 1H), 4.43-4.23 (m, 1H), 4.18-3.98 (m, 1H), 3.65-3.22 (m, 3H), 3.09 (s, 3H, 3.01-2.81 (m, 1H), 2.51-2.19 (m, 2H), 2.00-1.87 (m,1H) | 2.257 min, Chiral HPLC Method 7 | 2.398 min, LC/MS Method 19 | 471 |
| 38 | ((R)-3-(3,5- difluorophenyl) isoxazolidin-2- yl)((3S,4R)-3- fluoro-1-(5- (methylsulfonyl) pyrimidin-2- yl)piperidin-4- yl)methanone | 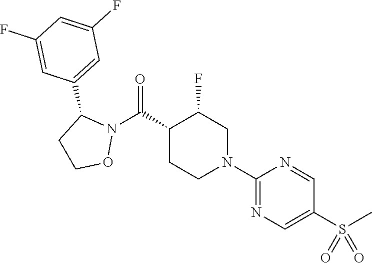 [SMILES: CS(=O)(=O)c1cnc(N2CC[C@H](C(=O)N3OCC[C@@H]3c3cc(F)cc(F)c3)[C@H](F)C2)nc1] | ¹H NMR (300 MHz, CDCl₃-d) ppm 8.73 (s, 2H), 6.97-6.79 (m, 2H), 6.79-6.62 (m, 1H), 5.42 (dd, J = 9.0, 6.0 Hz, 1H), 5.28-5.01 (m, 2H), 5.01-4.75 (m, 1H), 4.45-4.20 (m, 1H), 4.15-3.86 (m, 1H), 3.63-3.21 (m, 3H), 3.09 (s, 3H), 3.02-2.78 (m, 1H), 2.52-2.21 (m, 2H), 2.10-1.87 (m,1H) | 4.014 min, Chiral HPLC Method 7 | 2.399 min, LC/MS Method 22 | 471 |
| 39 | ((S)-3-(3,5- difluorophenyl) isoxazolidin-2- yl)((3S,4R)-3- fluoro-1-(5- (methylsulfonyl) pyrimidin-2- yl)piperidin-4- yl)methanone | 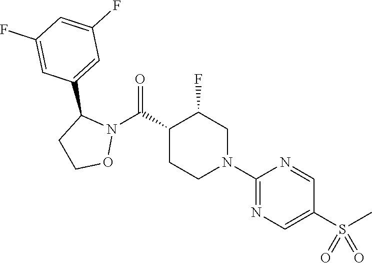 [SMILES: CS(=O)(=O)c1cnc(N2CC[C@H](C(=O)N3OCC[C@H]3c3cc(F)cc(F)c3)[C@H](F)C2)nc1] | ¹H NMR (300 MHz, CDCl₃-d) ppm 8.71 (S, 2H), 6.89-6.77 (m, 2H), 6.77-6.63 (m, 1H), 5.51-5.25 (m, 3H), 5.01 (dd, J = 15.0 Hz, 1H), 4.38-4.23 (m, 1H), 4.03-3.89 (m, 1H), 3.41-2.99 (m, 6H), 2.96-2.80 (m, 1H), 2.45-2.18 (m, 2H), 1.88-1.72 (m, 1H) | 1.873 min, Chiral HPLC Method 7 | 2.392 min, LC/MS Method 22 | 471 |
| 40 | ((R)-3-(3,5- difluorophenyl) isoxazolidin-2- yl)((3R,4S)-3- fluoro-1-(5- (methylsulfonyl) pyrimidin-2- yl)piperidin-4- yl)methanone | 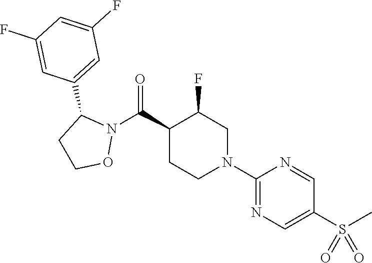 [SMILES: CS(=O)(=O)c1cnc(N2CC[C@@H](C(=O)N3OCC[C@@H]3c3cc(F)cc(F)c3)[C@@H](F)C2)nc1] | ¹H NMR (300 MHz, CDCl₃-d) ppm 8.70 (s, 2H), 6.85-6.75 (m, 2H), 6.73-6.61 (m, 1H), 5.50-5.25 (m, 3H), 5.10 (dd, J = 12.0 Hz, 1H), 4.35-4.22 (m, 1H), 4.05-3.89 (m, 1H), 3.41-2.99 (m, 6H), 2.97-2.80 (m, 1H), 2.45-2.18 (m, 2H), 1.88-1.73 (m, 1H) | 2.434 min, Chiral HPLC Method 7 | 2.383 min, LC/MS Method 22 | 471 |
| 41 | ((S)-3-(3,5- difluorophenyl) isoxazolidin-2- yl)((3R,4S)-3- fluoro-1-(6-((S)- methylsulfinyl) pyrimidin-4- yl)piperidin-4- yl)methanone | 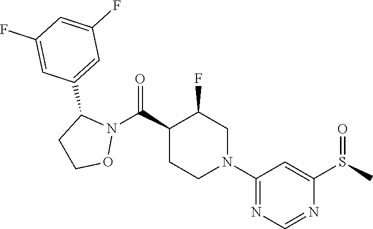 [SMILES: C[S@](=O)c1cc(N2CC[C@@H](C(=O)N3OCC[C@@H]3c3cc(F)cc(F)c3)[C@@H](F)C2)ncn1] | ¹H NMR (400 MHz, CDCl₃-d) ppm 8.53 (s, 1H), 7.24 (s, 1H), 6.88-6.80 (m, 2H), 6.76-6.68 (m, 1H), 5.44-5.36 (m, 1H), 5.14 (d, J = 47.2 Hz, 1H), 5.00-4.12 (m, 3H), 4.04-3.96 (m, 1H), 3.54-3.20 (m, 3H), 2.96-2.86 (m, 4H), 2.42-2.22 (m, 2H), 1.96-1.86 (m, 1H) | 2.406 min, Chiral HPLC Method 5 | 1.328 min, LC/MS Method 18 | 455 |
| 42 | ((R)-3-(3,5- difluorophenyl) isoxazolidin-2- yl)((3S,4R)-3- fluoro-1-(6-((S)- methylsulfinyl) pyrimidin-4- yl)piperidin-4- yl)methanone | 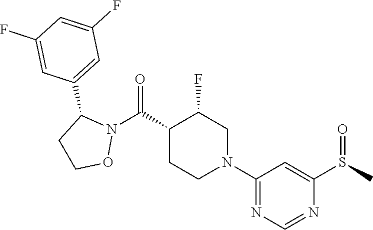 [SMILES: C[S@](=O)c1cc(N2CC[C@H](C(=O)N3OCC[C@@H]3c3cc(F)cc(F)c3)[C@H](F)C2)ncn1] | ¹H NMR (400 MHz, CDCl₃-d) ppm 8.53 (s, 1H), 7.24 (s, 1H), 6.88-6.80 (m, 2H), 6.76-6.68 (m, 1H), 5.48-5.38 (m, 1H), 5.14 (d, J = 47.6 Hz, 1H), 5.00-4.12 (m, 3H), 4.08-3.98 (m, 1H), 3.54-3.20 (m, 3H), 3.00-2.78 (m, 4H), 2.42-2.20 (m, 2H), 1.94-1.86 (m, 1H) | 2.846 min, Chiral HPLC Method 5 | 1.336 min, LC/MS Method 18 | 455 |
| 43 | ((S)-3-(3,5- difluorophenyl) isoxazolidin-2- yl)((3R,4S)-3- fluoro-1-(6-((R)- methylsulfinyl) pyrimidin-4- yl)piperidin-4- yl)methanone | 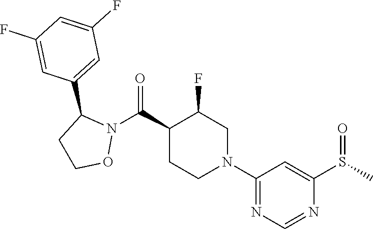 [SMILES: C[S@@](=O)c1cc(N2CC[C@@H](C(=O)N3OCC[C@H]3c3cc(F)cc(F)c3)[C@@H](F)C2)ncn1] | ¹H NMR (400 MHz, CDCl₃-d) ppm 8.53 (s, 1H), 7.24 (s, 1H), 6.88-6.80 (m, 2H), 6.78-6.68 (m, 1H), 5.46-5.38 (m, 1H), 5.14 (d, J = 47.6 Hz, 1H), 5.00-4.14 (m, 3H), 4.10-3.94 (m, 1H), 3.56-3.20 (m, 3H), 3.00-2.78 (m, 4H), 2.42-2.22 (m, 2H), 1.94-1.84 (m, 1H) | 3.375 min, Chiral HPLC Method 5 | 1.336 min, LC/MS Method 18 | 455 |
| 44 | ((R)-3-(3,5- difluorophenyl) isoxazolidin-2- yl)((3S,4R)-3- fluoro-1-(6-((R)- methylsulfinyl) pyrimidin-4- yl)piperidin-4- yl)methanone | 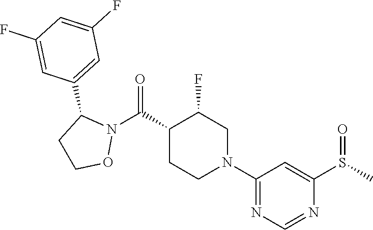 [SMILES: C[S@@](=O)c1cc(N2CC[C@H](C(=O)N3OCC[C@@H]3c3cc(F)cc(F)c3)[C@H](F)C2)ncn1] | 1H NMR (400 MHz, CDCl₃-d) ppm 8.53 (s, 1H), 7.25 (s, 1H), 6.88-6.80 (m, 2H), 6.78-6.68 (m, 1H), 5.46-5.38 (m, 1H), 5.14 (d, J = 47.6 Hz, 1H), 5.00-4.22 (m, 3H), 4.12-3.96 (m, 1H), 3.54-3.20 (m, 3H), 2.96-2.84 (m, 4H), 2.42-2.22 (m, 2H), 1.94-1.84 (m, 1H) | 4.175 min, Chiral HPLC Method 5 | 2.459 min, LC/MS Method 17 | 455 |
| 45 | ((S)-3-(3,5- difluorophenyl) isoxazolidin-2- yl)((3S,4R)-3- fluoro-1-(6-((S)- methylsulfinyl) pyrimidin-4- yl)piperidin-4- yl)methanone | 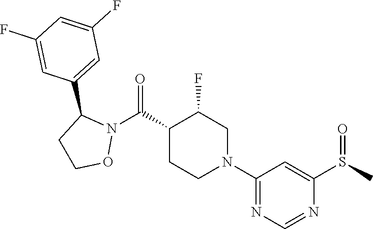 [SMILES: C[S@](=O)c1cc(N2CC[C@H](C(=O)N3OCC[C@H]3c3cc(F)cc(F)c3)[C@H](F)C2)ncn1] | ¹H NMR (400 MHz, CDCl₃-d) ppm 8.53 (s, 1H), 7.25 (s, 1H), 6.86-6.78 (m, 2H), 6.74-6.66 (m, 1H), 5.48-5.28 (m, 2H), 5.28-4.24 (m, 3H), 4.02-3.96 (m, 1H), 3.40-3.00 (m, 3H), 2.98-2.76 (m, 4H), 2.40-2.20 (m, 2H), 1.84-1.76 (m, 1H) | 3.029 min, Chiral HPLC Method 6 | 1.336 min, LC/MS Method 18 | 455 |
| 46 | ((R)-3-(3,5- difluorophenyl) isoxazolidin-2- yl)((3R,4S)-3- fluoro-1-(6-((S)- methylsulfinyl) pyrimidin-4- yl)piperidin-4- yl)methanone | 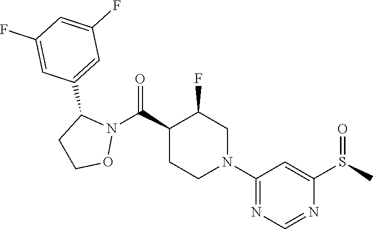 [SMILES: C[S@](=O)c1cc(N2CC[C@@H](C(=O)N3OCC[C@@H]3c3cc(F)cc(F)c3)[C@@H](F)C2)ncn1] | ¹H NMR (400 MHz, CDCl₃-d) ppm 8.53 (s, 1H), 7.25 (s, 1H), 6.86-6.78 (m, 2H), 6.74-6.68 (m, 1H), 5.46-5.28 (m, 2H), 5.24-4.10 (m, 3H), 4.02-3.82 (m, 1H), 3.44-3.00 (m, 3H), 2.98-2.74 (m, 4H), 2.40-2.20 (m, 2H), 1.84-1.76 (m, 1H) | 3.302 min, Chiral HPLC Method 6 | 1.336 min, LC/MS Method 18 | 455 |
| 47 | ((S)-3-(3,5- difluorophenyl) isoxazolidin-2- yl)((3S,4R)-3- fluoro-1-(6-((R)- methylsulfinyl) pyrimidin-4- yl)piperidin-4- yl)methanone | 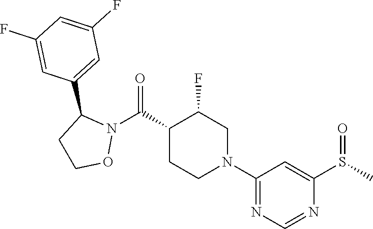 [SMILES: C[S@@](=O)c1cc(N2CC[C@H](C(=O)N3OCC[C@H]3c3cc(F)cc(F)c3)[C@H](F)C2)ncn1] | ¹H NMR (400 MHz, CDCl₃-d) ppm 8.53 (s, 1H), 7.25 (s, 1H), 6.86-6.78 (m, 2H), 6.74-6.68 (m, 1H), 5.46-5.28 (m, 2H), 5.28-4.22 (m, 3H), 4.00-3.90 (m, 1H), 3.40-3.00 (m, 3H), 2.92-2.80 (m, 4H), 2.40-2.20 (m 2H), 1.84-1.76 (m, 1H) | 3.551 min, Chiral HPLC Method 6 | 1.336 min, LC/MS Method 18 | 455 |
| 48 | ((R)-3-(3,5- difluorophenyl) isoxazolidin-2- yl)((3R,4S)-3- fluoro-1-(6-((R)- methylsulfinyl) pyrimidin-4- yl)piperidin-4- yl)methanone | 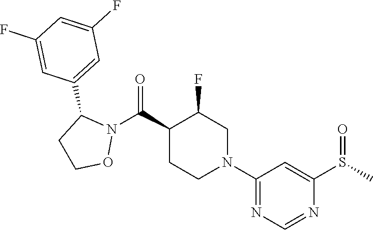 [SMILES: C[S@@](=O)c1cc(N2CC[C@@H](C(=O)N3OCC[C@@H]3c3cc(F)cc(F)c3)[C@@H](F)C2)ncn1] | ¹H NMR (400 MHz, CDCl₃-d) ppm 8.53 (s, 1H), 7.25 (s, 1H), 6.86-6.78 (m, 2H), 6.74-6.68 (m, 1H), 5.46-5.28 (m, 2H), 5.28-4.12 (m, 3H), 4.00-3.90 (m, 1H), 3.40-3.00 (m, 3H), 2.92-2.80 (m, 4H), 2.40-2.20 (m, 2H), 1.84-1.76 (m, 1H) | 3.844 min. Chiral HPLC Method 6 | 1.345 min, LC/MS Method 18 | 455 |

### Example 49 and Example 50

### cis-tert-butyl 3-fluoro-4-(3-(2,3,5-trifluorophenyl)isoxazolidine-2-carbonyl) piperidine-1-carboxylate

### and

### trans-tert-butyl 3-fluoro-4-(3-(2,3,5-trifluorophenyl)isoxazolidine-2-carbonyl) piperidine-1-carboxylate

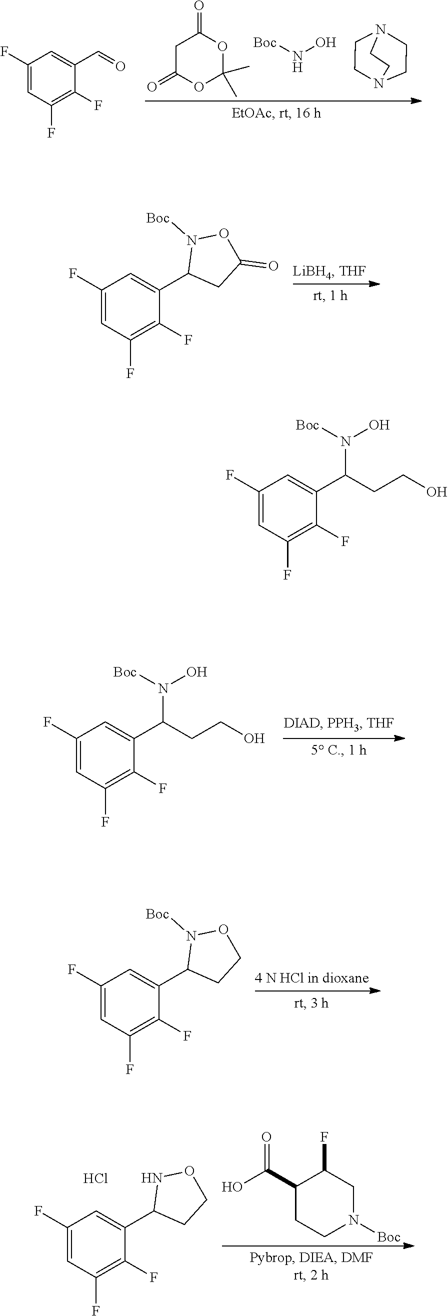

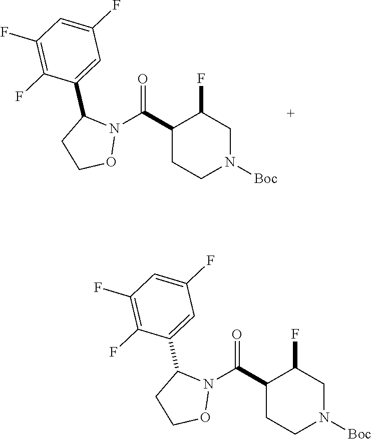

Step 1:

To a stirred solution of 2,3,5-trifluorobenzaldehyde (9 g, 56 mmol) in EA (100 mL) were added 2,2-dimethyl-1,3-dioxane-4,6-dione (8.06 g, 56 mmol), 1,4-diazabicyclo[2.2.2]octane (627 mg, 5.6 mmol), and tert-butyl N-hydroxycarbamate (7.45 g, 56 mmol). The reaction mixture was stirred at rt for 16 hours, diluted with H₂O (150 mL), and extracted with EA (3×50 mL). The combined organic layers were washed with brine (150 mL), dried over Na₂SO₄, filtered, and concentrated under vacuum to afford tert-butyl 5-oxo-3-(2,3,5-trifluorophenyl)isoxazolidine-2-carboxylate (17 g crude) as a yellow oil. LCMS (m/z) no mass signal, retention time: 1.208 min, LC/MS Method 13.

Step 2:

LiBH₄ (3.5 g, 161 mmol) was added into a stirred mixture of tert-butyl 5-oxo-3-(2,3,5-trifluorophenyl)isoxazolidine-2-carboxylate (17 g, 53.6 mmol) in THF (80 mL) in portions. The reaction mixture was stirred at rt for 1 hour, quenched by the addition of H₂O (150 mL), and extracted with EA (3×50 mL). The combined organic layers were washed with brine (150 mL), dried over anhydrous Na₂SO₄, filtered, and concentrated under vacuum to afford tert-butyl hydroxy(3-hydroxy-1-(2,3,5-trifluorophenyl)propyl)carbamate (17 g crude) as a light yellow solid. LCMS (m/z) 222 (M+H−100)⁺, retention time: 1.000 min, LC/MS Method 13.

Step 3:

DIAD (16 g, 80 mmol) was added into a stirred mixture of tert-butyl hydroxy(3-hydroxy-1-(2,3,5-trifluorophenyl)propyl)carbamate (17 g, 53 mmol) and PPh₃ (21 g, 80 mmol) in THF (80 mL) dropwise under nitrogen atmosphere at 5° C. The resulting mixture was stirred at 5° C. for 1 hour, quenched by the addition of H₂O (150 mL), and extracted with EA (3×80 mL). The combined organic layers were washed with brine (150 mL), dried over anhydrous Na₂SO₄, filtered, and concentrated under vacuum to afford tert-butyl 3-(2,3,5-trifluorophenyl)isoxazolidine-2-carboxylate (15 g, 88% yield for 3 steps) as a yellow oil. LCMS (m/z) 248 (M+H−56)⁺, retention time: 1.207 min, LC/MS Method 13.

Step 4:

Tert-butyl 3-(2,3,5-trifluorophenyl)isoxazolidine-2-carboxylate (15 g, 50 mmol) was added to a solution of HCl in dioxane (4 N, 100 mL). The resulting mixture was stirred at rt for 3 hours and concentrated under vacuum to afford 3-(2,3,5-trifluorophenyl)isoxazolidine hydrochloride (8.4 g crude) as a white solid. LCMS (m/z) 204 (M+H−37)⁺, retention time: 0.933 min, LC/MS Method 13.

Step 5:

3-(2,3,5-Trifluorophenyl)isoxazolidine hydrochloride (8 g, 33.3 mmol) was added to a stirred mixture of cis-1-(tert-butoxycarbonyl)-3-fluoropiperidine-4-carboxylic acid (8.2 g, 33.3 mmol), Pybrop (15.5 g, 33.3 mmol), and DIEA (8.6 g, 66.6 mmol) in DMF (60 mL). The resulting mixture was stirred for 2 hours at rt, quenched by the addition of H₂O (300 mL), and extracted with DCM (3×150 mL). The combined organic layers were washed with brine (300 mL), dried over Na₂SO₄, filtered, and concentrated under vacuum. The residue was purified by column chromatography (EA in PE from 0% to 50%) to afford two title compounds:

Example 49: Cis-tert-butyl-3-fluoro-4-(3-(2,3,5-trifluorophenyl)isoxazolidine-2-carbonyl)piperidine-1-carboxylate (2.3039 g, 32% yield) was obtained as a white solid, LCMS (m/z) 377 (M+H−56)⁺, retention time: 3.097 min, LC/MS Method 17. ¹H NMR (300 MHz, CDCl₃-d) ppm 6.90-6.74 (m, 2H), 5.70-5.60 (m, 1H), 5.12-4.82 (m, 1H), 4.44-3.92 (m, 4H), 3.30-2.90 (m, 4H), 2.40-2.10 (m, 2H), 1.84-1.66 (m, 1H), 1.46 (s, 9H).

Example 50: Trans-tert-butyl-3-fluoro-4-(3-(2,3,5-trifluorophenyl)isoxazolidine-2-carbonyl)piperidine-1-carboxylate (3.7681 g, 52% yield) was obtained as a white solid. LCMS (m/z) 377 (M+H−56)⁺, retention time: 1.773 min, LC/MS Method 18. ¹H NMR (300 MHz, CDCl₃-d) ppm 6.88-6.74 (m, 2H), 5.70-5.60 (m, 1H), 5.40-5.00 (m, 1H), 4.60-4.10 (m, 3H), 3.98-3.88 (m, 1H), 3.20-2.70 (m, 4H), 2.38-2.10 (m, 2H), 1.70-1.55 (m, 1H), 1.45 (s, 9H).

### Example 51 and Example 52

### Cis-tert-butyl 3-fluoro-4-(3-(5-fluoropyridin-3-yl)isoxazolidine-2-carbonyl)piperidine-1-carboxylate

### and

### trans-tert-butyl 3-fluoro-4-(3-(5-fluoropyridin-3-yl)isoxazolidine-2-carbonyl)piperidine-1-carboxylate

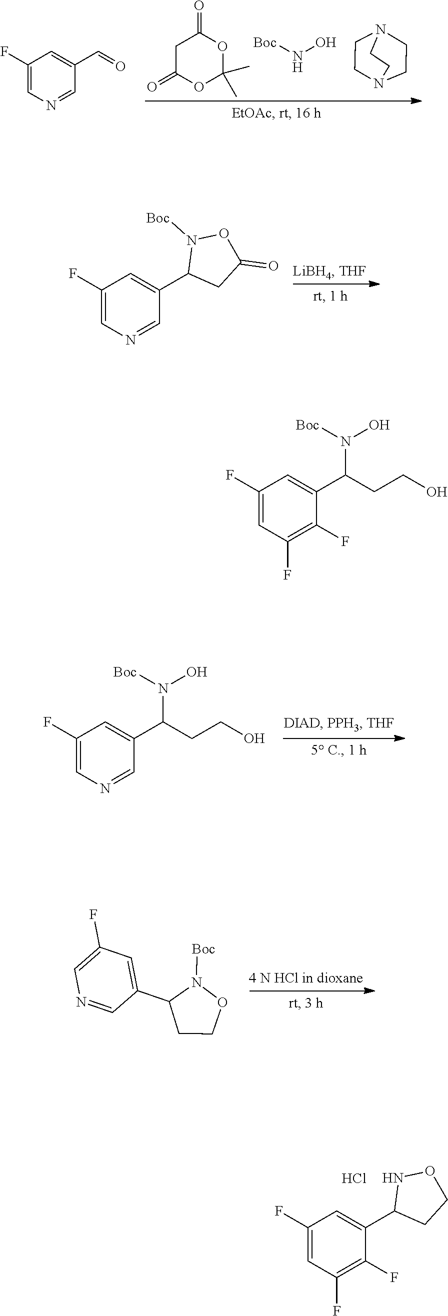

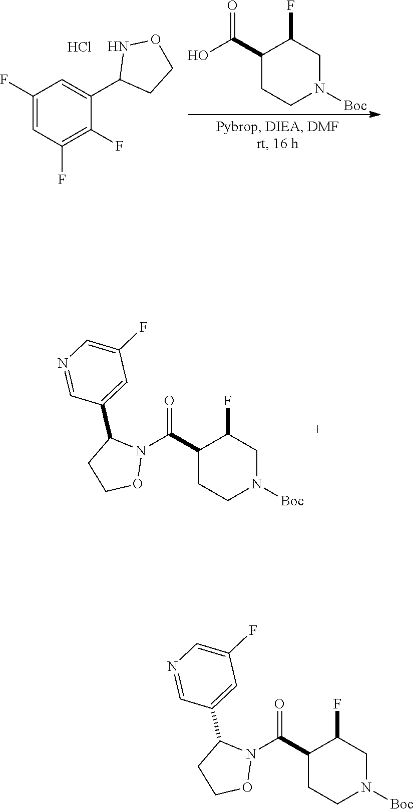

Step 1:

To a stirred solution of 5-fluoronicotinaldehyde (10 g, 80 mmol) in EA (200 mL) were added 2,2-dimethyl-1,3-dioxane-4,6-dione (11.52 g, 80 mmol), 1,4-diazabicyclo[2.2.2]octane (0.896 g, 8 mmol), and tert-butyl N-hydroxycarbamate (10.64 g, 80 mmol). The reaction mixture was stirred at rt for 16 hours, diluted with H₂O (200 mL), and extracted with EA (3×100 mL). The combined organic layers were washed with brine (150 mL), dried over anhydrous Na₂SO₄, filtered, and concentrated under vacuum to afford tert-butyl 3-(5-fluoropyridin-3-yl)-5-oxoisoxazolidine-2-carboxylate (18 g crude) as a yellow oil. LCMS (m/z) 283 (M+H)⁺, retention time: 0.941 min LC/MS Method 15.

Step 2:

LiBH₄ (4.21 g, 191.5 mmol) was added into a stirred mixture of tert-butyl 3-(5-fluoropyridin-3-yl)-5-oxoisoxazolidine-2-carboxylate (18 g, 63.8 mmol) in THF (300 mL) in portions. The reaction mixture was stirred at rt for 1 hour, quenched by the addition of H₂O (200 mL), and extracted with EA (3×300 mL). The combined organic layers were washed with brine (500 L), dried over anhydrous Na₂SO₄, filtered, and concentrated under vacuum to afford tert-butyl 1-(5-fluoropyridin-3-yl)-3-hydroxypropyl(hydroxy)carbamate (18 g crude) as a yellow oil. LCMS (m/z) 287 (M+H)⁺, retention time: 0.867 min, LC/MS Method 20.

Step 3:

DIAD (19 g, 94.4 mmol) was added into a stirred mixture of tert-butyl 1-(5-fluoropyridin-3-yl)-3-hydroxypropyl(hydroxy)carbamate (18 g, 62.9 mmol) and PPh₃ (24.7 g, 94.4 mmol) in THF (80 mL) dropwise under nitrogen atmosphere at 5° C. The resulting mixture was stirred at 5° C. for 1 hour, quenched by the addition of H₂O (150 mL), and extracted with EA (3×80 mL). The combined organic layers were washed with brine (200 mL), dried over anhydrous Na₂SO₄, and concentrated under vacuum. The residue was purified by column chromatography (EA in PE from 30% to 50%) to afford tert-butyl 3-(5-fluoropyridin-3-yl)isoxazolidine-2-carboxylate (5 g, 30% yield) as a brown oil. LCMS (m/z) 269 (M+H)⁺, retention time: 2.340 min, LC/MS Method 21. ¹H NMR (300 MHz, CDCl₃-d) ppm 8.45-8.41 (m, 2H), 7.52 (d, J=9 Hz, 1H), 5.34-5.29 (m, 1H), 4.24-4.20 (m, 1H), 3.96-3.88 (m, 1H), 2.89-2.86 (m, 1H), 2.32-2.29 (m, 1H), 1.52 (s, 9H).

Step 4:

Tert-butyl 3-(5-fluoropyridin-3-yl)isoxazolidine-2-carboxylate (1 g, 3.73 mmol) was added to a solution of HCl in dioxane (4 N, 10 mL). The resulting mixture was stirred at rt for 3 hours, and concentrated under vacuum to afford 3-(5-fluoropyridin-3-yl)isoxazolidine hydrochloride (800 g crude) as a white solid. LCMS (m/z) 169 (M+H)⁺, retention time: 0.524 min, LC/MS Method 13.

Step 5:

DIEA (1.01 g, 7.84 mmol) was added to a stirred mixture of cis-1-(tert-butoxycarbonyl)-3-fluoropiperidine-4-carboxylic acid (968 mg, 3.92 mmol) and Pybrop (1.83 g, 3.92 mmol) in DMF (10 mL). The resulting mixture was stirred at rt for 10 minutes, followed by addition of 3-(5-fluoropyridin-3-yl)isoxazolidine hydrochloride (800 mg, 3.92 mmol). The resulting mixture was stirred for 16 hours at rt, quenched by the addition of H₂O (50 mL), and extracted with EA (3×50 mL). The combined organic layers were washed with brine (20 mL), dried over anhydrous Na₂SO₄, and concentrated under vacuum. The residue was purified by column chromatography (EA in PE from 30% to 70%) to afford two title compounds:

Example 51: The crude cis-tert-butyl 3-fluoro-4-(3-(5-fluoropyridin-3-yl)isoxazolidine-2-carbonyl)piperidine-1-carboxylate was purified by Prep-HPLC with the condition: Column: XBridge Shield RP18 OBD Column, 5 um, 19×150 mm; Mobile Phase A: Water (10 mmol/L NH₄HCO₃), Mobile Phase B: ACN; Flow rate: 20 mL/min; Gradient: 25% B to 50% B in 12 min; UV 254 nm; Rt: 11.70 min to afford cis-tert-butyl 3-fluoro-4-(3-(5-fluoropyridin-3-yl)isoxazolidine-2-carbonyl)piperidine-1-carboxylate (236.2 mg, 15% yield) as a white solid, LCMS (m/z) 398 (M+H)⁺, retention time: 1.175 min, LC/MS Method 11. ¹H NMR (300 MHz, CDCl₃-d) ppm 8.43-8.41 (m, 2H), 7.36 (d, J=9 Hz, 1H), 5.53-5.50 (m, 1H), 4.96 (d, J=47.4 Hz, 1H), 4.35-4.25 (m, 2H), 4.20-4.00 (m, 2H), 3.25-3.10 (m, 2H), 3.01-2.91 (m, 2H), 2.44-2.37 (m, 1H), 2.20-2.10 (m, 1H), 1.84-1.66 (m, 1H), 1.49 (s, 9H).

Example 52: Trans-tert-butyl 3-fluoro-4-(3-(5-fluoropyridin-3-yl)isoxazolidine-2-carbonyl)piperidine-1-carboxylate (371.4 mg, 24% yield) was obtained as a white solid. LCMS (m/z) 3 98 (M+H)⁺, retention time: 1.405 min, LC/MS Method 8. ¹H NMR (300 MHz, CDCl₃-d) ppm 8.41-8.37 (m, 2H), 7.38-7.34 (m, 1H), 5.55-5.50 (m, 1H), 5.20 (d, J=47.4 Hz, 1H), 4.39-4.10 (m, 2H), 3.99-3.91 (m, 1H), 3.20-2.90 (m, 3H), 2.87-2.70 (m, 2H), 2.45-2.33 (m, 1H), 2.28-2. 13 (m, 1H), 1.66-1.60 (m, 1H), 1.47 (s, 9H).

### Pharmaceutical Compositions

EXAMPLE A—An ointment is prepared by combining 20% (w/w) of any compound of Examples 1-52; and 80% (w/w) of petrolatum. The mixture is passed through a roller mill until a uniform consistency is obtained.

EXAMPLE B—Aerosol Spray: A solution is prepared from the following components: [Ingredient (Amount (w/w))]: A compound of Example 1-52 (1.00); propylene glycol (5.00); golysorbate 80 (1.00); ethanol (78.00); and purified water (15.00). The solution is placed in a conventional aerosol container, a valve mechanism is attached, and the container is charged with nitrogen to 100 psig.

EXAMPLE C—Tablets are prepared using conventional methods and are formulated as follows: [Ingredient (Amount per tablet)]: A compound of Example 1-52 (5 mg); microcrystalline cellulose (100 mg); lactose (100 mg); sodium starch glycollate (30 mg); and magnesium stearate (2 mg).

EXAMPLE D—Capsules are prepared using conventional methods and are formulated as follows: [Ingredient (Amount per tablet)]: A compound of Example 1-52 (15 mg); dried starch (178 mg); and magnesium stearate (2 mg).

### Biological Assays

Biological In Vitro Cell Assay

The efficacy of RIP1 inhibitors can be tested in mice in vitro using a human monocytic leukemia U937 in a necroptosis assay. As determined using the method described in S. He et al., Cell, 137(6):1100-1111 (2009) and International Patent Appln. No. PCT/IB2014/059004, now, International Patent Appln. Pub. No. WO2014/125444, the compounds of Examples 1-52 exhibited a pIC₅₀ between approximately 5.0 and 9.0.

For instance, the compounds of Examples 2, 4, 5, 8, 9, 10, 12, 14, 16, 18, 22, 23, 26, 30, 32, 34, 35, 38, 40, 44, and 46 inhibited necrosis in U937 cells in the above method with a pIC₅₀ between approximately 6.0 and 9.0.

For instance, the compounds of Examples 2, 4, 5, 8, 9, 10, 16, 18, 22, 26, 30, 32, 34, 35, 44, and 46 inhibited necrosis in U937 cells in the above method with a pIC₅₀ between approximately 7.0 and 9.0.

Viability was measured by quantitating cellular levels of ATP using the Cell Titer-Glo kit. All data are means±standard deviation of the mean.

1. A compound according to Formula (I) or pharmaceutically acceptable salt thereof: 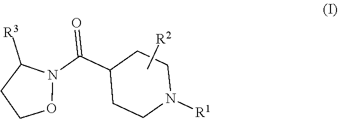
wherein:
R¹ is —CO(C₁-C₄)alkyl, —CO₂(C₁-C₄)alkyl, or 5-6 membered heteroaryl group,
wherein said 5-6 membered heteroaryl group is optionally substituted by 1 or 2 substituents independently selected from cyano, (C₁-C₄)alkyl, —CONH₂, —CONH((C₁-C₄)alkyl), —CON((C₁-C₄)alkyl)((C₁-C₄)alkyl), —SO(C₁-C₄)alkyl, and —SO₂(C₁-C₄)alkyl;
R² is hydrogen or a halogen; and
R³ is phenyl or 5-6 membered heteroaryl group,
wherein said phenyl or 5-6 membered heteroaryl group is optionally substituted by 1, 2, or 3 halogens.

2. The compound or a pharmaceutically acceptable salt thereof according to claim 1, wherein:
R¹ is furyl, thienyl, pyrrolyl, imidazolyl, pyrazolyl, triazolyl, tetrazolyl, thiazolyl, isothiazolyl, thiadiazolyl, oxazolyl, isoxazolyl, oxadiazolyl, oxo-oxadiazolyl, pyridinyl, oxo-pyridyl, pyrazinyl, pyrimidinyl, pyridazinyl, or triazinyl,
wherein said substituted furyl, thienyl, pyrrolyl, imidazolyl, pyrazolyl, triazolyl, tetrazolyl, thiazolyl, isothiazolyl, thiadiazolyl, oxazolyl, isoxazolyl, oxadiazolyl, oxo-oxadiazolyl, pyridinyl, oxo-pyridyl, pyrazinyl, pyrimidinyl, pyridazinyl and triazinyl is optionally substituted by one substituent selected from cyano, (C₁-C₄)alkyl, —CONH₂, —CONH((C₁-C₄)alkyl), —CON((C₁-C₄)alkyl)((C₁-C₄)alkyl), —SO(C₁-C₄)alkyl, and —SO₂(C₁-C₄)alkyl.

3. The compound or a pharmaceutically acceptable salt thereof according to claim 1, wherein:
R¹ is thiadiazolyl, oxadiazolyl, or pyrimidinyl,
wherein thiadiazolyl, oxadiazolyl, or pyrimidinyl is optionally substituted by one substituent selected from cyano, (C₁-C₄)alkyl, —CONH₂, —CONH((C₁-C₄)alkyl), and —CON((C₁-C₄)alkyl)((C₁-C₄)alkyl).

4. The compound or a pharmaceutically acceptable salt thereof according to claim 1, wherein:
R¹ is thiadiazolyl, oxadiazolyl, or pyrimidinyl,
wherein thiadiazolyl, oxadiazolyl, or pyrimidinyl is optionally substituted by one substituent selected from cyano, methyl, —CONH₂, —CONHCH₃, and —CON(CH₃)₂.

5. The compound or pharmaceutically acceptable salt thereof according to claim 1, wherein R² is hydrogen or fluoro.

6. The compound or pharmaceutically acceptable salt thereof according to claim 1, wherein R³ is phenyl or 5-6 membered heteroaryl group, wherein said phenyl or 5-6 membered heteroaryl group is optionally substituted by one, two, or three fluoros.

7. The compound or pharmaceutically acceptable salt thereof according to claim 1, wherein R³ is phenyl or pyridyl, wherein said phenyl or pyridinyl is optionally substituted by one, two, or three fluoros.

8. The compound, or a pharmaceutically acceptable salt thereof, according to claim 1, having Formula (II):

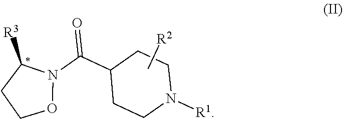

9. The compound according to claim 1 which is:
1-(4-(3-(3,5-difluorophenyl)isoxazolidine-2-carbonyl)piperidin-1-yl)ethan-1-one;
(R)-1-(4-(3-(3,5-difluorophenyl)isoxazolidine-2-carbonyl)piperidin-1-yl)ethan-1-one;
(S)-1-(4-(3-(3,5-difluorophenyl)isoxazolidine-2-carbonyl)piperidin-1-yl)ethan-1-one;
6-(4-(3-(3,5-difluorophenyl)isoxazolidine-2-carbonyl)piperidin-1-yl)pyrimidine-4-carboxamide;
(R)-6-(4-(3-(3,5-difluorophenyl)isoxazolidine-2-carbonyl)piperidin-1-yl)pyrimidine-4-carboxamide;
(S)-6-(4-(3-(3,5-difluorophenyl)isoxazolidine-2-carbonyl)piperidin-1-yl)pyrimidine-4-carboxamide;
(3-(3,5-difluorophenyl)isoxazolidin-2-yl)(1-(5-methyl-1,3,4-oxadiazol-2-yl)piperidin-4-yl)methanone;
(R)-(3-(3,5-difluorophenyl)isoxazolidin-2-yl)(1-(5-methyl-1,3,4-oxadiazol-2-yl)piperidin-4-yl)methanone;
(S)-(3-(3,5-difluorophenyl)isoxazolidin-2-yl)(1-(5-methyl-1,3,4-oxadiazol-2-yl)piperidin-4-yl)methanone;
6-(4-(3-(3,5-difluorophenyl)isoxazolidine-2-carbonyl)piperidin-1-yl)pyrimidine-4-carbonitrile;
(R)-6-(4-(3-(3,5-difluorophenyl)isoxazolidine-2-carbonyl)piperidin-1-yl)pyrimidine-4-carbonitrile;
(S)-6-(4-(3-(3,5-difluorophenyl)isoxazolidine-2-carbonyl)piperidin-1-yl)pyrimidine-4-carbonitrile;
6-(4-(3-(3,5-difluorophenyl)isoxazolidine-2-carbonyl)piperidin-1-yl)-N-methylpyrimidine-4-carboxamide;
(R)-6-(4-(3-(3,5-difluorophenyl)isoxazolidine-2-carbonyl)piperidin-1-yl)-N-methylpyrimidine-4-carboxamide;
(S)-6-(4-(3-(3,5-difluorophenyl)isoxazolidine-2-carbonyl)piperidin-1-yl)-N-methylpyrimidine-4-carboxamide;
6-(4-(3-(3,5-difluorophenyl)isoxazolidine-2-carbonyl)piperidin-1-yl)-N,N-dimethylpyrimidine-4-carboxamide;
(R)-6-(4-(3-(3,5-difluorophenyl)isoxazolidine-2-carbonyl)piperidin-1-yl)-N,N-dimethylpyrimidine-4-carboxamide;
(S)-6-(4-(3-(3,5-difluorophenyl)isoxazolidine-2-carbonyl)piperidin-1-yl)-N,N-dimethylpyrimidine-4-carboxamide;
1-((3R,4S)-4-((S)-3-(3,5-difluorophenyl)isoxazolidine-2-carbonyl)-3-fluoropiperidin-1-yl)ethan-1-one;
1-((3S,4R)-4-((R)-3-(3,5-difluorophenyl)isoxazolidine-2-carbonyl)-3-fluoropiperidin-1-yl)ethan-1-one;
1-((3S,4R)-4-((S)-3-(3,5-difluorophenyl)isoxazolidine-2-carbonyl)-3-fluoropiperidin-1-yl)ethan-1-one;
1-((3R,4S)-4-((R)-3-(3,5-difluorophenyl)isoxazolidine-2-carbonyl)-3-fluoropiperidin-1-yl)ethan-1-one;
((S)-3-(3,5-difluorophenyl)isoxazolidin-2-yl)((3R,4S)-3-fluoro-1-(5-methyl-1,3,4-oxadiazol-2-yl)piperidin-4-yl)methanone;
((R)-3-(3,5-difluorophenyl)isoxazolidin-2-yl)((3 S,4R)-3-fluoro-1-(5-methyl-1,3,4-oxadiazol-2-yl)piperidin-4-yl)methanone;
((S)-3-(3,5-difluorophenyl)isoxazolidin-2-yl)((3 S,4R)-3-fluoro-1-(5-methyl-1,3,4-oxadiazol-2-yl)piperidin-4-yl)methanone;
((R)-3-(3,5-difluorophenyl)isoxazolidin-2-yl)((3R,4S)-3-fluoro-1-(5-methyl-1,3,4-oxadiazol-2-yl)piperidin-4-yl)methanone;
((S)-3-(3,5-difluorophenyl)isoxazolidin-2-yl)((3 S,4R)-3-fluoro-1-(5-methyl-1,3,4-thiadiazol-2-yl)piperidin-4-yl)methanone;
((R)-3-(3,5-difluorophenyl)isoxazolidin-2-yl)((3R,4S)-3-fluoro-1-(5-methyl-1,3,4-thiadiazol-2-yl)piperidin-4-yl)methanone;
((S)-3-(3,5-difluorophenyl)isoxazolidin-2-yl)((3R,4S)-3-fluoro-1-(1,3,4-oxadiazol-2-yl)piperidin-4-yl)methanone;
((R)-3-(3,5-difluorophenyl)isoxazolidin-2-yl)((3 S,4R)-3-fluoro-1-(1,3,4-oxadiazol-2-yl)piperidin-4-yl)methanone;
((S)-3-(3,5-difluorophenyl)isoxazolidin-2-yl)((3 S,4R)-3-fluoro-1-(1,3,4-oxadiazol-2-yl)piperidin-4-yl)methanone;
((R)-3-(3,5-difluorophenyl)isoxazolidin-2-yl)((3R,4S)-3-fluoro-1-(1,3,4-oxadiazol-2-yl)piperidin-4-yl)methanone;
6-((3R,4S)-4-((S)-3-(3,5-difluorophenyl)isoxazolidine-2-carbonyl)-3-fluoropiperidin-1-yl)pyrimidine-4-carboxamide;
6-((3 S,4R)-4-((R)-3-(3,5-difluorophenyl)isoxazolidine-2-carbonyl)-3-fluoropiperidin-1-yl)pyrimidine-4-carboxamide;
6-((3 S,4R)-4-((S)-3-(3,5-difluorophenyl)isoxazolidine-2-carbonyl)-3-fluoropiperidin-1-yl)pyrimidine-4-carboxamide;
6-((3R,4S)-4-((R)-3-(3,5-difluorophenyl)isoxazolidine-2-carbonyl)-3-fluoropiperidin-1-yl)pyrimidine-4-carboxamide;
6-((3R,4S)-4-((S)-3-(3,5-difluorophenyl)isoxazolidine-2-carbonyl)-3-fluoropiperidin-1-yl)-N-methylpyrimidine-4-carboxamide;
6-((3 S,4R)-4-((R)-3-(3,5-difluorophenyl)isoxazolidine-2-carbonyl)-3-fluoropiperidin-1-yl)-N-methylpyrimidine-4-carboxamide;
6-((3 S,4R)-4-((S)-3-(3,5-difluorophenyl)isoxazolidine-2-carbonyl)-3-fluoropiperidin-1-yl)-N-methylpyrimidine-4-carboxamide;
6-((3R,4S)-4-((R)-3-(3,5-difluorophenyl)isoxazolidine-2-carbonyl)-3-fluoropiperidin-1-yl)-N-methylpyrimidine-4-carboxamide;
6-((3R,4S)-4-((S)-3-(3,5-difluorophenyl)isoxazolidine-2-carbonyl)-3-fluoropiperidin-1-yl)-N,N-dimethylpyrimidine-4-carboxamide;
6-((3 S,4R)-4-((R)-3-(3,5-difluorophenyl)isoxazolidine-2-carbonyl)-3-fluoropiperidin-1-yl)-N,N-dimethylpyrimidine-4-carboxamide;
6-((3 S,4R)-4-((S)-3-(3,5-difluorophenyl)isoxazolidine-2-carbonyl)-3-fluoropiperidin-1-yl)-N,N-dimethylpyrimidine-4-carboxamide;
6-((3R,4S)-4-((R)-3-(3,5-difluorophenyl)isoxazolidine-2-carbonyl)-3-fluoropiperidin-1-yl)-N,N-dimethylpyrimidine-4-carboxamide;
((S)-3-(3,5-difluorophenyl)isoxazolidin-2-yl)((3R,4S)-3-fluoro-1-(5-(methylsulfonyl)pyrimidin-2-yl)piperidin-4-yl)methanone;
((R)-3-(3,5-difluorophenyl)isoxazolidin-2-yl)((3 S,4R)-3-fluoro-1-(5-(methylsulfonyl)pyrimidin-2-yl)piperidin-4-yl)methanone;
((S)-3-(3,5-difluorophenyl)isoxazolidin-2-yl)((3 S,4R)-3-fluoro-1-(5-(methylsulfonyl)pyrimidin-2-yl)piperidin-4-yl)methanone;
((R)-3-(3,5-difluorophenyl)isoxazolidin-2-yl)((3R,4S)-3-fluoro-1-(5-(methylsulfonyl)pyrimidin-2-yl)piperidin-4-yl)methanone;
((S)-3-(3,5-difluorophenyl)isoxazolidin-2-yl)((3R,4S)-3-fluoro-1-(6-((S)-methylsulfinyl)pyrimidin-4-yl)piperidin-4-yl)methanone;
((R)-3-(3,5-difluorophenyl)isoxazolidin-2-yl)((3S,4R)-3-fluoro-1-(6-((S)-methylsulfinyl)pyrimidin-4-yl)piperidin-4-yl)methanone;
((S)-3-(3,5-difluorophenyl)isoxazolidin-2-yl)((3R,4S)-3-fluoro-1-(6-((R)-methylsulfinyl)pyrimidin-4-yl)piperidin-4-yl)methanone;
((R)-3-(3,5-difluorophenyl)isoxazolidin-2-yl)((3S,4R)-3-fluoro-1-(6-((R)-methylsulfinyl)pyrimidin-4-yl)piperidin-4-yl)methanone;
((S)-3-(3,5-difluorophenyl)isoxazolidin-2-yl)((3S,4R)-3-fluoro-1-(6-((S)-methylsulfinyl)pyrimidin-4-yl)piperidin-4-yl)methanone;
((R)-3-(3,5-difluorophenyl)isoxazolidin-2-yl)((3R,4S)-3-fluoro-1-(6-((S)-methylsulfinyl)pyrimidin-4-yl)piperidin-4-yl)methanone;
((S)-3-(3,5-difluorophenyl)isoxazolidin-2-yl)((3S,4R)-3-fluoro-1-(6-((R)-methylsulfinyl)pyrimidin-4-yl)piperidin-4-yl)methanone;
((R)-3-(3,5-difluorophenyl)isoxazolidin-2-yl)((3R,4S)-3-fluoro-1-(6-((R)-methylsulfinyl)pyrimidin-4-yl)piperidin-4-yl)methanone;
cis-tert-butyl 3-fluoro-4-(3-(2,3,5-trifluorophenyl)isoxazolidine-2-carbonyl)piperidine-1-carboxylate;
trans-tert-butyl 3-fluoro-4-(3-(2,3,5-trifluorophenyl)isoxazolidine-2-carbonyl)piperidine-1-carboxylate;
cis-tert-butyl 3-fluoro-4-(3-(5-fluoropyridin-3-yl)isoxazolidine-2-carbonyl)piperidine-1-carboxylate; and
trans-tert-butyl 3-fluoro-4-(3-(5-fluoropyridin-3-yl)isoxazolidine-2-carbonyl)piperidine-1-carboxylate;
or pharmaceutically acceptable salts thereof.

10. A pharmaceutical composition comprising the compound or pharmaceutically acceptable salt thereof according to claim 1 and a pharmaceutically acceptable excipient.

11. The pharmaceutical composition according to claim 10, which further comprises at least one other therapeutically active agent.

12. A method of treating a RIP1 kinase-mediated disease or disorder in a human in need thereof comprising administering to the human a therapeutically effective amount of the compound or pharmaceutically acceptable salt thereof according to claim 1.

13. The method of treating RIP1 kinase-mediated disease or disorder in a human in need thereof according to claim 12, wherein the RIP1 kinase-mediated disease or disorder is ulcerative colitis, psoriasis, rheumatoid arthritis, or amyotrophic lateral sclerosis.

14.-24. (canceled)
